(19) United States
(12) Patent Application Publication
(10) Pub. No.: US 2023/0339890 A1
(43) Pub. Date: Oct. 26, 2023

(54) AMINO ALCOHOL COMPOUNDS AND USES THEREOF

(71) Applicant: Kineta, Inc., Seattle, WA (US)

(72) Inventors: Iwona WRONA, Sharon, MA (US); Kerem OZBOYA, Cambridge, MA (US); Bertrand LE BOURDONNEC, Northborough, MA (US); Vanessa KURIA, Somerville, MA (US); Madeline MACDONNELL, Brighton, MA (US); Matthew LUCAS, Lexington, MA (US); Byron DELABARRE, Arlington, MA (US)

(21) Appl. No.: 18/007,812

(22) PCT Filed: Jun. 3, 2021
(86) PCT No.: PCT/US2021/035765

§ 371 Date: Dec. 2, 2022

Related U.S. Application Data
(60) Provisional application No. 63/034,089, filed on Jun. 3, 2020.

(51) Int. Cl. C07D 401/12; C07D 213/30; C07D 405/12; C07D 405/14; C07D 237/08; C07D 417/12; C07D 241/12; C07D 401/14

(52) U.S. Cl. CPC C07D 401/12; C07D 213/30; C07D 237/08; C07D 241/12; C07D 401/14; C07D 405/12; C07D 405/14; C07D 417/12

(57) ABSTRACT

The present invention features compounds useful in the treatment of neurological disorders. The compounds of the invention, alone or in combination with other pharmaceutically active agents, can be used for treating or preventing neurological disorders.

### BACKGROUND

An incomplete understanding of the molecular perturbations that cause disease, as well as a limited arsenal of robust model systems, has contributed to a failure to generate successful disease-modifying therapies against common and progressive neurological disorders, such as ALS and FTD. Progress is being made on many fronts to find agents that can arrest the progress of these disorders. However, the present therapies for most, if not all, of these diseases provide very little relief. Accordingly, a need exists to develop therapies that can alter the course of neurodegenerative diseases. More generally, a need exists for better methods and compositions for the treatment of neurodegenerative diseases in order to improve the quality of the lives of those afflicted by such diseases.

### SUMMARY

TDP-43 is a nuclear DNA/RNA binding protein involved in RNA splicing. Under pathological cell stress, TDP-43 translocates to the cytoplasm and aggregates into stress granules. These phenotypes are hallmarks of degenerating motor neurons and are found in 97% of all ALS cases. The highly penetrant nature of this pathology indicates that TDP-43 is broadly involved in both familial and sporadic ALS. Additionally, TDP-43 mutations that promote aggregation are linked to higher risk of developing ALS, suggesting protein misfolding and aggregation act as drivers of toxicity. TDP-43 toxicity can be recapitulated in yeast models, where the protein induces a viability deficit and localizes to stress granules. The present inventors have discovered that the CYP51A1 inhibitors described herein are capable of reversing TDP-43 induced toxicity. Accordingly, the present invention describes such CYP51A1 inhibitors and methods of using these compounds for the treatment of disorders related to TDP-43 toxicity such as ALS.

In an aspect, the invention features a compound, or a pharmaceutically acceptable salt thereof, having the structure:

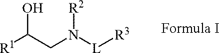

wherein R¹ has the structure:

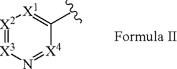

X¹, _{X}², X³, and X⁴ are, independently, N or CR⁴, wherein at least three of X¹, _{X}², X³, and X⁴ are CR⁴;
R² is optionally substituted C₁-C₆ alkyl, optionally substituted C₁-C₆ alkoxy, optionally substituted C₃-C₈ cycloalkyl, optionally substituted C₂-C₉ heterocylcyl, optionally substituted C₂-C₉ heteroaryl, optionally substituted C₁-C₆ alkyl C₃-C₈ cycloalkyl, optionally substituted C₁-C₆ alkyl C₂-C₉ heterocylcyl, optionally substituted C₁-C₆ alkyl C₂-C₉ heteroaryl, or optionally substituted C₁-C₆ alkyl C₆-C₁₀ aryl;
R³ is optionally substituted C₁-C₆ alkyl, optionally substituted C₁-C₆ alkoxy, optionally substituted C₃-C₈ cycloalkyl, optionally substituted C₂-C₉ heterocylcyl, optionally substituted C₂-C₉ heteroaryl, optionally substituted C₁-C₆ alkyl C₃-C₈ cycloalkyl, optionally substituted C₁-C₆ alkyl C₂-C₉ heterocylcyl, optionally substituted C₁-C₆ alkyl C₂-C₉ heteroaryl, or optionally substituted C₁-C₆ alkyl C₆-C₁₀ aryl, wherein if R² is methyl then R³ is optionally substituted C₁-C₆ alkyl, optionally substituted C₁-C₆ alkoxy, optionally substituted C₃-C₈ cycloalkyl, or optionally substituted C₁-C₆ alkyl C₃-C₈ cycloalkyl;
each R⁴ is, independently, hydrogen, hydroxy, halo, cyano, optionally substituted amino, optionally substituted C₁-C₆ alkyl, or optionally substituted C₁-C₆ alkoxy; and
L is —C(O)— or —SO₂—.

In some embodiments, L is —C(O)—. In some embodiments, L is —SO₂—.

In some embodiments, X¹, _{X}², X³, and X⁴ are CR⁴. In some embodiments, R¹ has the structure:

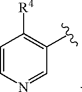

In some embodiments, R⁴ is optionally substituted C₁-C₆ alkyl (e.g., methyl or iso-propyl). In some embodiments, R⁴ is optionally substituted amino (e.g., —NH₂). In some embodiments, R⁴ is optionally substituted C₁-C₆ alkoxy (e.g., methoxy).

In some embodiments, R¹ has the structure:

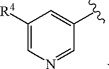

In some embodiments, R⁴ is halo (e.g., fluoro, bromo, or chloro). In some embodiments, R⁴ is optionally substituted C₁-C₆ alkyl (e.g., —C(O)CH₃ or

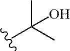

). In some embodiments, R⁴ is cyano.

In some embodiments, R¹ has the structure:

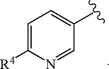

In some embodiments, R⁴ is optionally substituted C₁-C₆ alkyl (e.g., trifluoromethyl). In some embodiments, R⁴ is hydroxy. In some embodiments, R⁴ is optionally substituted C₁-C₆ alkoxy (e.g., methoxy).

In some embodiments, X¹ is N and _{X}², X³, and X⁴ are CR⁴. In some embodiments, R⁴ is hydrogen.

In some embodiments, X¹, _{X}², X⁴ are CR⁴, and X³ is N. In some embodiments, R⁴ is hydrogen.

In some embodiments, X¹, _{X}², X³ are CR⁴, and X⁴ is N. In some embodiments, R⁴ is hydrogen.

In some embodiments, R² is optionally substituted C₁-C₆ alkyl (e.g., ethyl, propyl, iso-propyl, or —C(O)CH₂CH₃).

In some embodiments, R³ is optionally substituted C₁-C₆ alkyl C₆-C₁₀ aryl (e.g., 3,4-chloro-benzyl, 2-fluoro-4-chloro-benzyl, 3-fluoro-4-trifluoromethyl-benzyl, 3-chloro-4-methyl-benzyl, 3-chloro-4-trifluoromethoxy-benzyl, 3-fluoro-4-difluoromethyl-benzyl, or napthyl). In some embodiments, R³ is optionally substituted C₁-C₆ alkyl C₂-C₉ heteroaryl (e.g.,

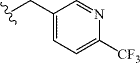

). In some embodiments, R³ is optionally substituted C₃-C₈ cycloalkyl (e.g.,

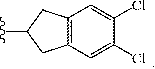

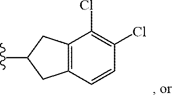

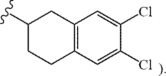

In an aspect, the invention features a compound, or a pharmaceutically acceptable salt thereof, having the structure of any one of compounds 1-147 in Table 1.

TABLE 1

Compounds of the Invention

| # | Structure | # | Structure |
|---|---|---|---|
| 1 | 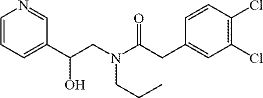 [SMILES: CCCN(CC(O)c1cccnc1)C(=O)Cc1ccc(Cl)c(Cl)c1] | 75 | 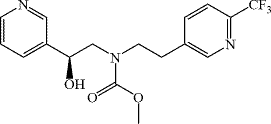 [SMILES: COC(=O)N(CCc1ccc(C(F)(F)F)nc1)C[C@@H](O)c1cccnc1] |
| 2 | 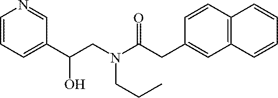 [SMILES: CCCN(CC(O)c1cccnc1)C(=O)Cc1ccc2ccccc2c1] | 76 | 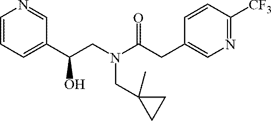 [SMILES: CC1(CN(C[C@@H](O)c2cccnc2)C(=O)Cc2ccc(C(F)(F)F)nc2)CC1] |
| 3 | 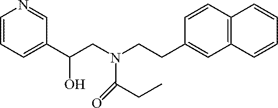 [SMILES: CCC(=O)N(CCc1ccc2ccccc2c1)CC(O)c1cccnc1] | 77 | 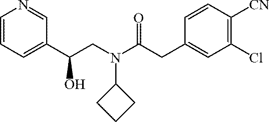 [SMILES: N#Cc1ccc(CC(=O)N(C[C@@H](O)c2cccnc2)C2CCC2)cc1Cl] |
| 4 | 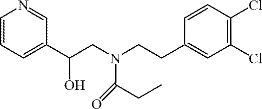 [SMILES: CCC(=O)N(CCc1ccc(Cl)c(Cl)c1)CC(O)c1cccnc1] | 78 | 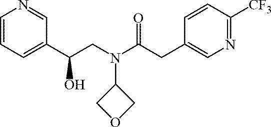 [SMILES: O=C(Cc1ccc(C(F)(F)F)nc1)N(C[C@@H](O)c1cccnc1)C1COC1] |
| 5 | 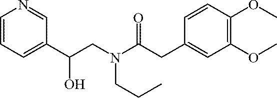 [SMILES: CCCN(CC(O)c1cccnc1)C(=O)Cc1ccc(OC)c(OC)c1] | 79 | 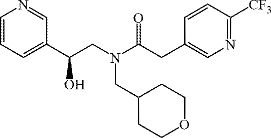 [SMILES: O=C(Cc1ccc(C(F)(F)F)nc1)N(CC1CCOCC1)C[C@@H](O)c1cccnc1] |
| 6 | 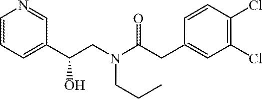 [SMILES: CCCN(C[C@H](O)c1cccnc1)C(=O)Cc1ccc(Cl)c(Cl)c1] | 80 | 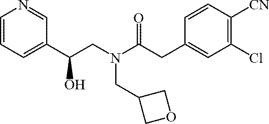 [SMILES: N#Cc1ccc(CC(=O)N(CC2COC2)C[C@@H](O)c2cccnc2)cc1Cl] |
| 7 | 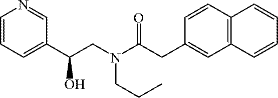 [SMILES: CCCN(C[C@@H](O)c1cccnc1)C(=O)Cc1ccc2ccccc2c1] | 81 | 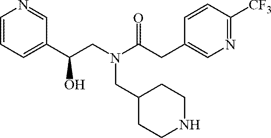 [SMILES: O=C(Cc1ccc(C(F)(F)F)nc1)N(CC1CCNCC1)C[C@@H](O)c1cccnc1] |
| 8 | 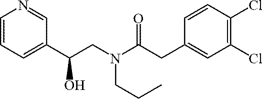 [SMILES: CCCN(C[C@@H](O)c1cccnc1)C(=O)Cc1ccc(Cl)c(Cl)c1] | 82 | 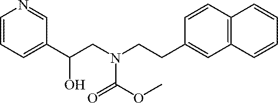 [SMILES: COC(=O)N(CCc1ccc2ccccc2c1)CC(O)c1cccnc1] |
| 9 | 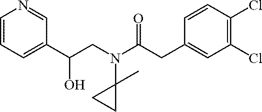 [SMILES: CC1(N(CC(O)c2cccnc2)C(=O)Cc2ccc(Cl)c(Cl)c2)CC1] | 83 | 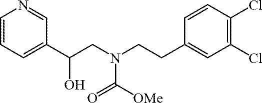 [SMILES: COC(=O)N(CCc1ccc(Cl)c(Cl)c1)CC(O)c1cccnc1] |
| 10 | 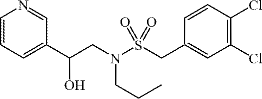 [SMILES: CCCN(CC(O)c1cccnc1)S(=O)(=O)Cc1ccc(Cl)c(Cl)c1] | 84 | 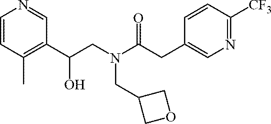 [SMILES: Cc1ccncc1C(O)CN(CC1COC1)C(=O)Cc1ccc(C(F)(F)F)nc1] |
| 11 | 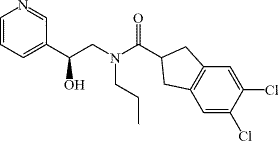 [SMILES: CCCN(C[C@@H](O)c1cccnc1)C(=O)C1Cc2cc(Cl)c(Cl)cc2C1] | 85 | 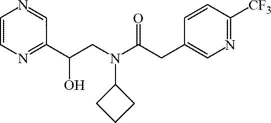 [SMILES: O=C(Cc1ccc(C(F)(F)F)nc1)N(CC(O)c1cnccn1)C1CCC1] |
| 12 | 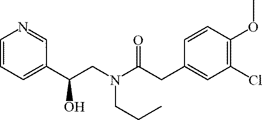 [SMILES: CCCN(C[C@@H](O)c1cccnc1)C(=O)Cc1ccc(OC)c(Cl)c1] | 86 | 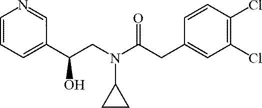 [SMILES: O=C(Cc1ccc(Cl)c(Cl)c1)N(C[C@@H](O)c1cccnc1)C1CC1] |
| 13 | 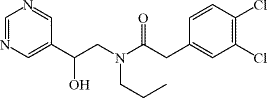 [SMILES: CCCN(CC(O)c1cncnc1)C(=O)Cc1ccc(Cl)c(Cl)c1] | 87 | 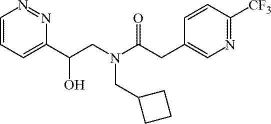 [SMILES: O=C(Cc1ccc(C(F)(F)F)nc1)N(CC1CCC1)CC(O)c1cccnn1] |
| 14 | 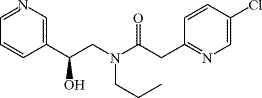 [SMILES: CCCN(C[C@@H](O)c1cccnc1)C(=O)Cc1ccc(Cl)cn1] | 88 | 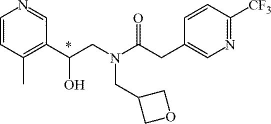 |
| 15 | 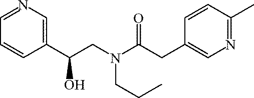 [SMILES: CCCN(C[C@@H](O)c1cccnc1)C(=O)Cc1ccc(C)nc1] | 89 | 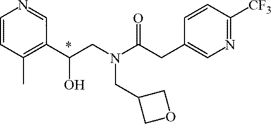 |
| 16 | 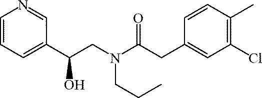 [SMILES: CCCN(C[C@@H](O)c1cccnc1)C(=O)Cc1ccc(C)c(Cl)c1] | 90 | 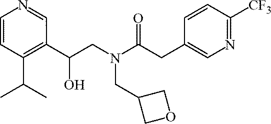 [SMILES: CC(C)c1ccncc1C(O)CN(CC1COC1)C(=O)Cc1ccc(C(F)(F)F)nc1] |
| 17 | 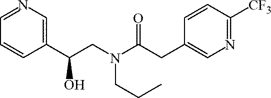 [SMILES: CCCN(C[C@@H](O)c1cccnc1)C(=O)Cc1ccc(C(F)(F)F)nc1] | 91 | 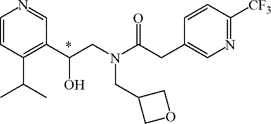 |
| 18 | 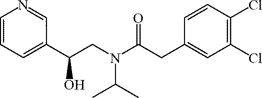 [SMILES: CC(C)N(C[C@@H](O)c1cccnc1)C(=O)Cc1ccc(Cl)c(Cl)c1] | 92 | 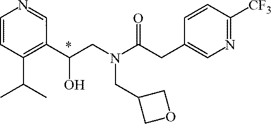 |
| 19 | 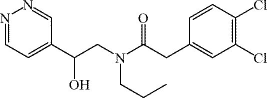 [SMILES: CCCN(CC(O)c1ccnnc1)C(=O)Cc1ccc(Cl)c(Cl)c1] | 93 | 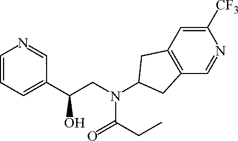 [SMILES: CCC(=O)N(C[C@@H](O)c1cccnc1)C1Cc2cnc(C(F)(F)F)cc2C1] |
| 20 | 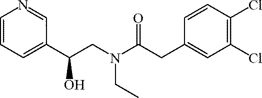 [SMILES: CCN(C[C@@H](O)c1cccnc1)C(=O)Cc1ccc(Cl)c(Cl)c1] | 94 | 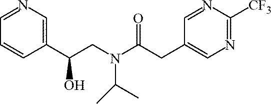 [SMILES: CC(C)N(C[C@@H](O)c1cccnc1)C(=O)Cc1cnc(C(F)(F)F)nc1] |
| 21 | 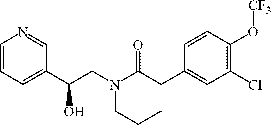 [SMILES: CCCN(C[C@@H](O)c1cccnc1)C(=O)Cc1ccc(OC(F)(F)F)c(Cl)c1] | 95 | 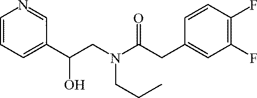 [SMILES: CCCN(CC(O)c1cccnc1)C(=O)Cc1ccc(F)c(F)c1] |
| 22 | 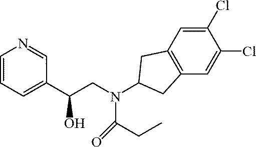 [SMILES: CCC(=O)N(C[C@@H](O)c1cccnc1)C1Cc2cc(Cl)c(Cl)cc2C1] | 96 | 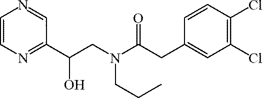 [SMILES: CCCN(CC(O)c1cnccn1)C(=O)Cc1ccc(Cl)c(Cl)c1] |
| 23 | 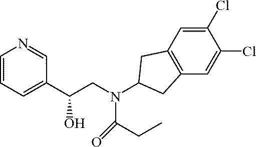 [SMILES: CCC(=O)N(C[C@H](O)c1cccnc1)C1Cc2cc(Cl)c(Cl)cc2C1] | 97 | 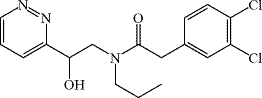 [SMILES: CCCN(CC(O)c1cccnn1)C(=O)Cc1ccc(Cl)c(Cl)c1] |
| 24 | 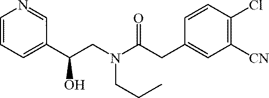 [SMILES: CCCN(C[C@@H](O)c1cccnc1)C(=O)Cc1ccc(Cl)c(C#N)c1] | 98 | 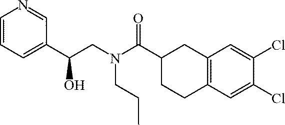 [SMILES: CCCN(C[C@@H](O)c1cccnc1)C(=O)C1CCc2cc(Cl)c(Cl)cc2C1] |
| 25 | 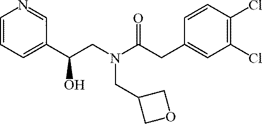 [SMILES: O=C(Cc1ccc(Cl)c(Cl)c1)N(CC1COC1)C[C@@H](O)c1cccnc1] | 99 | 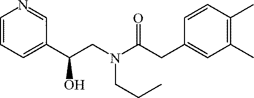 [SMILES: CCCN(C[C@@H](O)c1cccnc1)C(=O)Cc1ccc(C)c(C)c1] |
| 26 | 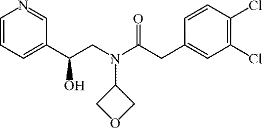 [SMILES: O=C(Cc1ccc(Cl)c(Cl)c1)N(C[C@@H](O)c1cccnc1)C1COC1] | 100 | 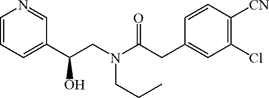 [SMILES: CCCN(C[C@@H](O)c1cccnc1)C(=O)Cc1ccc(C#N)c(Cl)c1] |
| 27 | 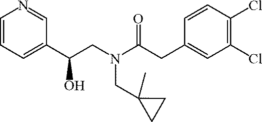 [SMILES: CC1(CN(C[C@@H](O)c2cccnc2)C(=O)Cc2ccc(Cl)c(Cl)c2)CC1] | 101 | 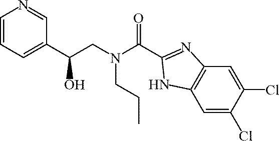 [SMILES: CCCN(C[C@@H](O)c1cccnc1)C(=O)c1nc2cc(Cl)c(Cl)cc2[nH]1] |
| 28 | 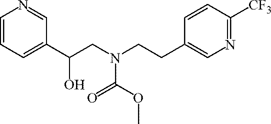 [SMILES: COC(=O)N(CCc1ccc(C(F)(F)F)nc1)CC(O)c1cccnc1] | 102 | 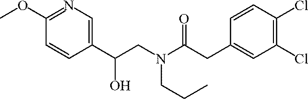 [SMILES: CCCN(CC(O)c1ccc(OC)nc1)C(=O)Cc1ccc(Cl)c(Cl)c1] |
| 29 | 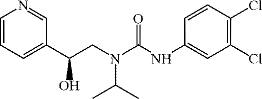 [SMILES: CC(C)N(C[C@@H](O)c1cccnc1)C(=O)Nc1ccc(Cl)c(Cl)c1] | 103 | 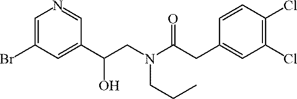 [SMILES: CCCN(CC(O)c1cncc(Br)c1)C(=O)Cc1ccc(Cl)c(Cl)c1] |
| 30 | 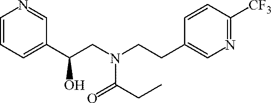 [SMILES: CCC(=O)N(CCc1ccc(C(F)(F)F)nc1)C[C@@H](O)c1cccnc1] | 104 | 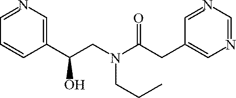 [SMILES: CCCN(C[C@@H](O)c1cccnc1)C(=O)Cc1cncnc1] |
| 31 | 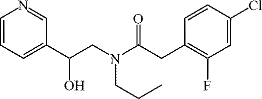 [SMILES: CCCN(CC(O)c1cccnc1)C(=O)Cc1ccc(Cl)cc1F] | 105 | 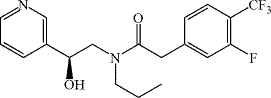 [SMILES: CCCN(C[C@@H](O)c1cccnc1)C(=O)Cc1ccc(C(F)(F)F)c(F)c1] |
| 32 | 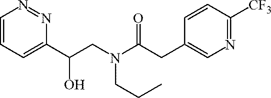 [SMILES: CCCN(CC(O)c1cccnn1)C(=O)Cc1ccc(C(F)(F)F)nc1] | 106 | 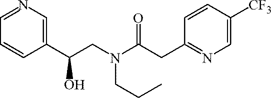 [SMILES: CCCN(C[C@@H](O)c1cccnc1)C(=O)Cc1ccc(C(F)(F)F)cn1] |
| 33 | 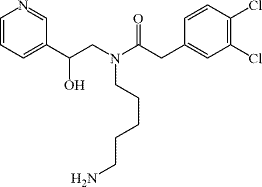 [SMILES: NCCCCCN(CC(O)c1cccnc1)C(=O)Cc1ccc(Cl)c(Cl)c1] | 107 | 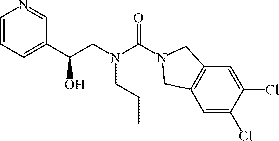 [SMILES: CCCN(C[C@@H](O)c1cccnc1)C(=O)N1Cc2cc(Cl)c(Cl)cc2C1] |
| 34 | 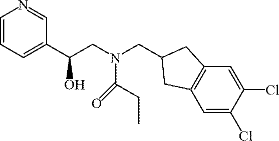 [SMILES: CCC(=O)N(CC1Cc2cc(Cl)c(Cl)cc2C1)C[C@@H](O)c1cccnc1] | 108 | 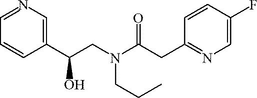 [SMILES: CCCN(C[C@@H](O)c1cccnc1)C(=O)Cc1ccc(F)cn1] |
| 35 | 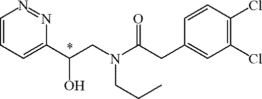 | 109 | 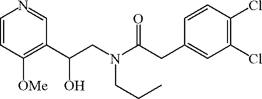 [SMILES: CCCN(CC(O)c1cnccc1OC)C(=O)Cc1ccc(Cl)c(Cl)c1] |
| 36 | 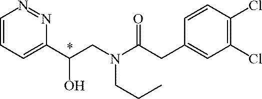 | 110 | 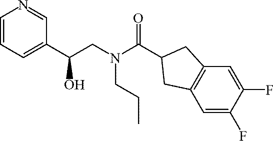 [SMILES: CCCN(C[C@@H](O)c1cccnc1)C(=O)C1Cc2cc(F)c(F)cc2C1] |
| 37 | 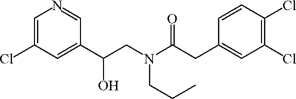 [SMILES: CCCN(CC(O)c1cncc(Cl)c1)C(=O)Cc1ccc(Cl)c(Cl)c1] | 111 | 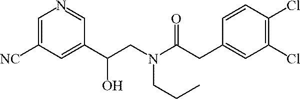 [SMILES: CCCN(CC(O)c1cncc(C#N)c1)C(=O)Cc1ccc(Cl)c(Cl)c1] |
| 38 | 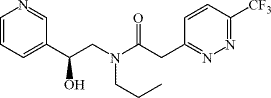 [SMILES: CCCN(C[C@@H](O)c1cccnc1)C(=O)Cc1ccc(C(F)(F)F)nn1] | 112 | 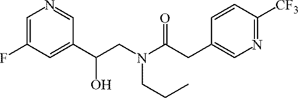 [SMILES: CCCN(CC(O)c1cncc(F)c1)C(=O)Cc1ccc(C(F)(F)F)nc1] |
| 39 | 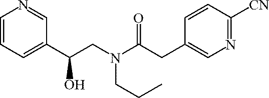 [SMILES: CCCN(C[C@@H](O)c1cccnc1)C(=O)Cc1ccc(C#N)nc1] | 113 | 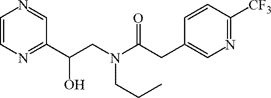 [SMILES: CCCN(CC(O)c1cnccn1)C(=O)Cc1ccc(C(F)(F)F)nc1] |
| 40 | 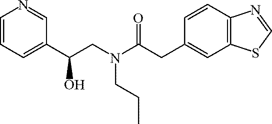 [SMILES: CCCN(C[C@@H](O)c1cccnc1)C(=O)Cc1ccc2ncsc2c1] | 114 | 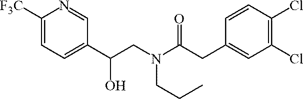 [SMILES: CCCN(CC(O)c1ccc(C(F)(F)F)nc1)C(=O)Cc1ccc(Cl)c(Cl)c1] |
| 41 | 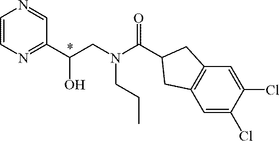 | 115 | 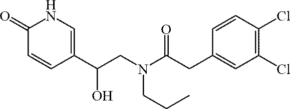 [SMILES: CCCN(CC(O)c1ccc(=O)[nH]c1)C(=O)Cc1ccc(Cl)c(Cl)c1] |
| 42 | 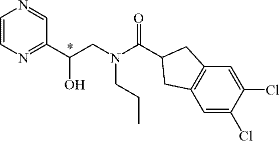 | 116 | 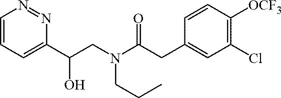 [SMILES: CCCN(CC(O)c1cccnn1)C(=O)Cc1ccc(OC(F)(F)F)c(Cl)c1] |
| 43 | 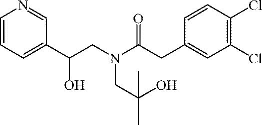 [SMILES: CC(C)(O)CN(CC(O)c1cccnc1)C(=O)Cc1ccc(Cl)c(Cl)c1] | 117 | 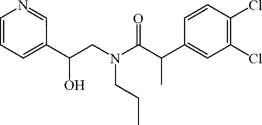 [SMILES: CCCN(CC(O)c1cccnc1)C(=O)C(C)c1ccc(Cl)c(Cl)c1] |
| 44 | 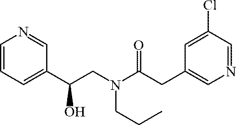 [SMILES: CCCN(C[C@@H](O)c1cccnc1)C(=O)Cc1cncc(Cl)c1] | 118 | 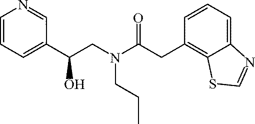 [SMILES: CCCN(C[C@@H](O)c1cccnc1)C(=O)Cc1cccc2ncsc12] |
| 45 | 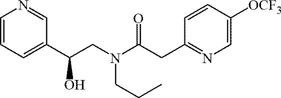 [SMILES: CCCN(C[C@@H](O)c1cccnc1)C(=O)Cc1ccc(OC(F)(F)F)cn1] | 119 | 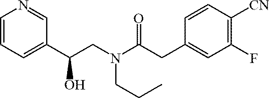 [SMILES: CCCN(C[C@@H](O)c1cccnc1)C(=O)Cc1ccc(C#N)c(F)c1] |
| 46 | 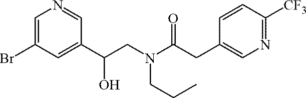 [SMILES: CCCN(CC(O)c1cncc(Br)c1)C(=O)Cc1ccc(C(F)(F)F)nc1] | 120 | 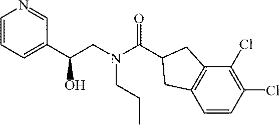 [SMILES: CCCN(C[C@@H](O)c1cccnc1)C(=O)C1Cc2ccc(Cl)c(Cl)c2C1] |
| 47 | 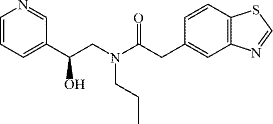 [SMILES: CCCN(C[C@@H](O)c1cccnc1)C(=O)Cc1ccc2scnc2c1] | 121 | 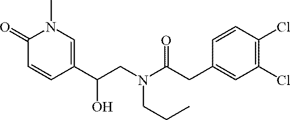 [SMILES: CCCN(CC(O)c1ccc(=O)n(C)c1)C(=O)Cc1ccc(Cl)c(Cl)c1] |
| 48 | 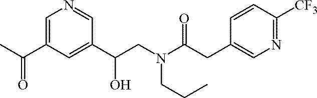 [SMILES: CCCN(CC(O)c1cncc(C(C)=O)c1)C(=O)Cc1ccc(C(F)(F)F)nc1] | 122 | 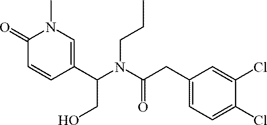 [SMILES: CCCN(C(=O)Cc1ccc(Cl)c(Cl)c1)C(CO)c1ccc(=O)n(C)c1] |
| 49 | 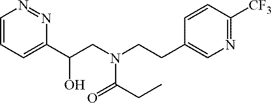 [SMILES: CCC(=O)N(CCc1ccc(C(F)(F)F)nc1)CC(O)c1cccnn1] | 123 | 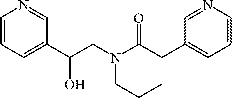 [SMILES: CCCN(CC(O)c1cccnc1)C(=O)Cc1cccnc1] |
| 50 | 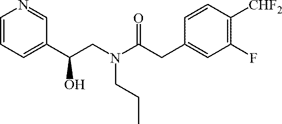 [SMILES: CCCN(C[C@@H](O)c1cccnc1)C(=O)Cc1ccc(C(F)F)c(F)c1] | 124 | 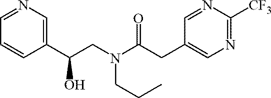 [SMILES: CCCN(C[C@@H](O)c1cccnc1)C(=O)Cc1cnc(C(F)(F)F)nc1] |
| 51 | 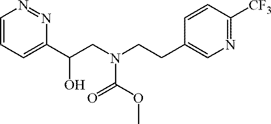 [SMILES: COC(=O)N(CCc1ccc(C(F)(F)F)nc1)CC(O)c1cccnn1] | 125 | 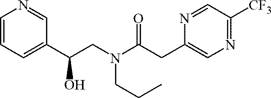 [SMILES: CCCN(C[C@@H](O)c1cccnc1)C(=O)Cc1cnc(C(F)(F)F)cn1] |
| 52 | 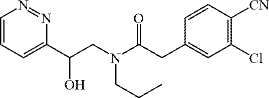 [SMILES: CCCN(CC(O)c1cccnn1)C(=O)Cc1ccc(C#N)c(Cl)c1] | 126 | 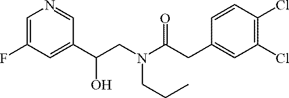 [SMILES: CCCN(CC(O)c1cncc(F)c1)C(=O)Cc1ccc(Cl)c(Cl)c1] |
| 53 | 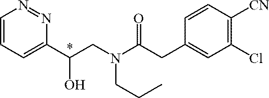 | 127 | 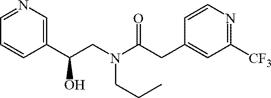 [SMILES: CCCN(C[C@@H](O)c1cccnc1)C(=O)Cc1ccnc(C(F)(F)F)c1] |
| 54 | 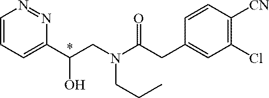 | 128 | 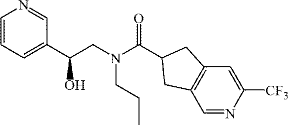 [SMILES: CCCN(C[C@@H](O)c1cccnc1)C(=O)C1Cc2cnc(C(F)(F)F)cc2C1] |
| 55 | 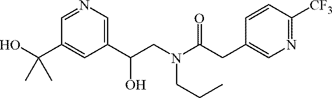 [SMILES: CCCN(CC(O)c1cncc(C(C)(C)O)c1)C(=O)Cc1ccc(C(F)(F)F)nc1] | 129 | 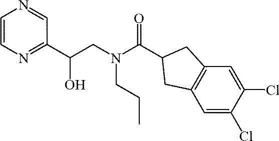 [SMILES: CCCN(CC(O)c1cnccn1)C(=O)C1Cc2cc(Cl)c(Cl)cc2C1] |
| 56 | 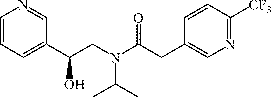 [SMILES: CC(C)N(C[C@@H](O)c1cccnc1)C(=O)Cc1ccc(C(F)(F)F)nc1] | 130 | 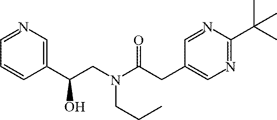 [SMILES: CCCN(C[C@@H](O)c1cccnc1)C(=O)Cc1cnc(C(C)(C)C)nc1] |
| 57 | 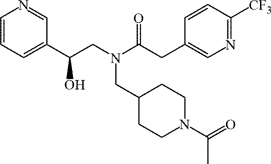 [SMILES: CC(=O)N1CCC(CN(C[C@@H](O)c2cccnc2)C(=O)Cc2ccc(C(F)(F)F)nc2)CC1] | 131 | 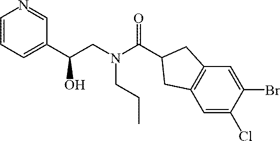 [SMILES: CCCN(C[C@@H](O)c1cccnc1)C(=O)C1Cc2cc(Cl)c(Br)cc2C1] |
| 58 | 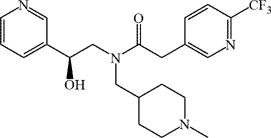 [SMILES: CN1CCC(CN(C[C@@H](O)c2cccnc2)C(=O)Cc2ccc(C(F)(F)F)nc2)CC1] | 132 | 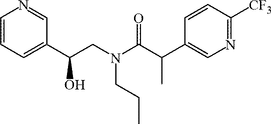 [SMILES: CCCN(C[C@@H](O)c1cccnc1)C(=O)C(C)c1ccc(C(F)(F)F)nc1] |
| 59 | 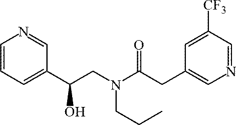 [SMILES: CCCN(C[C@@H](O)c1cccnc1)C(=O)Cc1cncc(C(F)(F)F)c1] | 133 | 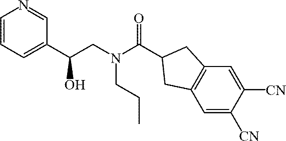 [SMILES: CCCN(C[C@@H](O)c1cccnc1)C(=O)C1Cc2cc(C#N)c(C#N)cc2C1] |
| 60 | 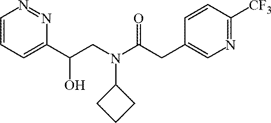 [SMILES: O=C(Cc1ccc(C(F)(F)F)nc1)N(CC(O)c1cccnn1)C1CCC1] | 134 | 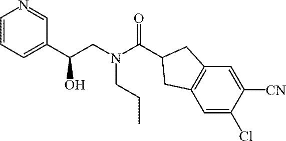 [SMILES: CCCN(C[C@@H](O)c1cccnc1)C(=O)C1Cc2cc(Cl)c(C#N)cc2C1] |
| 61 | 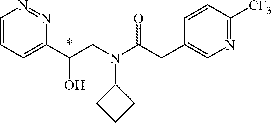 | 135 | 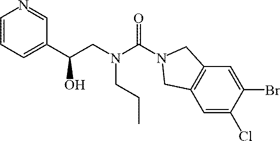 [SMILES: CCCN(C[C@@H](O)c1cccnc1)C(=O)N1Cc2cc(Cl)c(Br)cc2C1] |
| 62 | 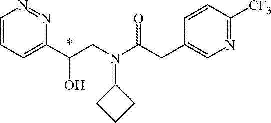 | 136 | 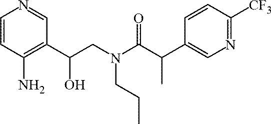 [SMILES: CCCN(CC(O)c1cnccc1N)C(=O)C(C)c1ccc(C(F)(F)F)nc1] |
| 63 | 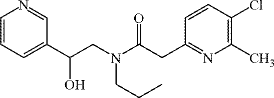 [SMILES: CCCN(CC(O)c1cccnc1)C(=O)Cc1ccc(Cl)c(C)n1] | 137 | 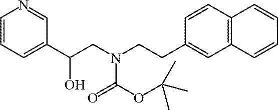 [SMILES: CC(C)(C)OC(=O)N(CCc1ccc2ccccc2c1)CC(O)c1cccnc1] |
| 64 | 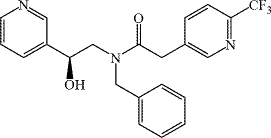 [SMILES: O=C(Cc1ccc(C(F)(F)F)nc1)N(Cc1ccccc1)C[C@@H](O)c1cccnc1] | 138 | 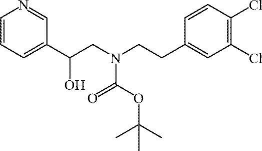 [SMILES: CC(C)(C)OC(=O)N(CCc1ccc(Cl)c(Cl)c1)CC(O)c1cccnc1] |
| 65 | 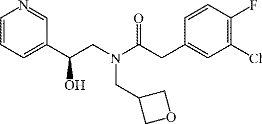 [SMILES: O=C(Cc1ccc(F)c(Cl)c1)N(CC1COC1)C[C@@H](O)c1cccnc1] | 139 | 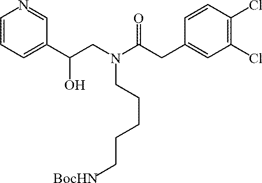 [SMILES: CC(C)(C)OC(=O)NCCCCCN(CC(O)c1cccnc1)C(=O)Cc1ccc(Cl)c(Cl)c1] |
| 66 | 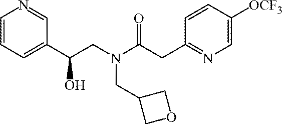 [SMILES: O=C(Cc1ccc(OC(F)(F)F)cn1)N(CC1COC1)C[C@@H](O)c1cccnc1] | 140 | 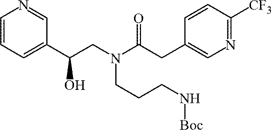 [SMILES: CC(C)(C)OC(=O)NCCCN(C[C@@H](O)c1cccnc1)C(=O)Cc1ccc(C(F)(F)F)nc1] |
| 67 | 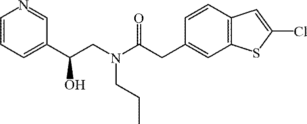 [SMILES: CCCN(C[C@@H](O)c1cccnc1)C(=O)Cc1ccc2cc(Cl)sc2c1] | 141 | 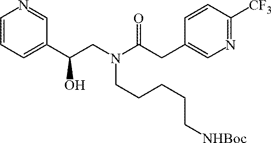 [SMILES: CC(C)(C)OC(=O)NCCCCCN(C[C@@H](O)c1cccnc1)C(=O)Cc1ccc(C(F)(F)F)nc1] |
| 68 | 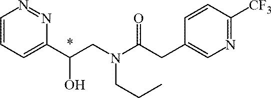 | 142 | 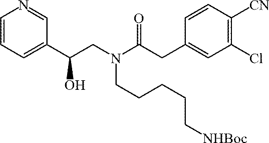 [SMILES: CC(C)(C)OC(=O)NCCCCCN(C[C@@H](O)c1cccnc1)C(=O)Cc1ccc(C#N)c(Cl)c1] |
| 69 | 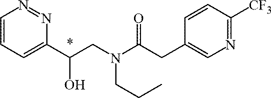 | 143 | 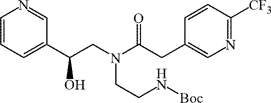 [SMILES: CC(C)(C)OC(=O)NCCN(C[C@@H](O)c1cccnc1)C(=O)Cc1ccc(C(F)(F)F)nc1] |
| 70 | 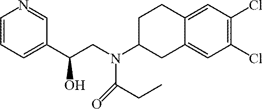 [SMILES: CCC(=O)N(C[C@@H](O)c1cccnc1)C1CCc2cc(Cl)c(Cl)cc2C1] | 144 | 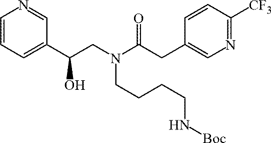 [SMILES: CC(C)(C)OC(=O)NCCCCN(C[C@@H](O)c1cccnc1)C(=O)Cc1ccc(C(F)(F)F)nc1] |
| 71 | 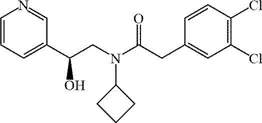 [SMILES: O=C(Cc1ccc(Cl)c(Cl)c1)N(C[C@@H](O)c1cccnc1)C1CCC1] | 145 | 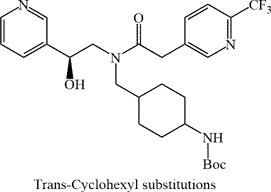 |
| 72 | 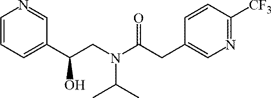 [SMILES: CC(C)N(C[C@@H](O)c1cccnc1)C(=O)Cc1ccc(C(F)(F)F)nc1] | 146 | 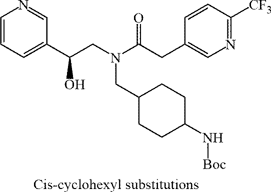 |
| 73 | 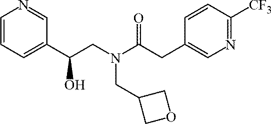 [SMILES: O=C(Cc1ccc(C(F)(F)F)nc1)N(CC1COC1)C[C@@H](O)c1cccnc1] | 147 | 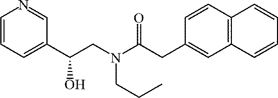 [SMILES: CCCN(C[C@H](O)c1cccnc1)C(=O)Cc1ccc2ccccc2c1] |
| 74 | 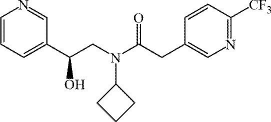 [SMILES: O=C(Cc1ccc(C(F)(F)F)nc1)N(C[C@@H](O)c1cccnc1)C1CCC1] | | |

In an aspect, the invention features a pharmaceutical composition comprising any of the foregoing compounds and a pharmaceutically acceptable excipient.

In an aspect, the invention features a method of treating a neurological disorder (e.g., frontotemporal dementia (FTLD-TDP), chronic traumatic encephalopathy, ALS, Alzheimer’s disease, limbic-predominant age-related TDP-42 encephalopathy (LATE), or frontotemporal lobar degeneration) in a subject in need thereof. This method includes administering an effective amount of any of the foregoing compounds or pharmaceutical compositions.

In an aspect, the invention features a method of inhibiting toxicity in a cell (e.g., mammalian neural cell) related to a protein (e.g., TDP-43). This method includes administering an effective amount of any of the foregoing compounds or pharmaceutical compositions.

In an aspect, the invention features a method of treating a CYP51A1-associated disorder (e.g., FTLD-TDP, chronic traumatic encephalopathy, ALS, Alzheimer’s disease, LATE, or frontotemporal lobar degeneration) in a subject in need thereof. This method includes administering an effective amount of any of the foregoing compounds pharmaceutical compositions.

In an aspect, the invention features a method of inhibiting CYP51A1. This method includes contacting a cell with an effective amount of any of the foregoing compounds or pharmaceutical compositions.

In another aspect, the invention features a method of treating a neurological disorder in a patient, such as a human patient, identified as likely to benefit from treatment with a CYP51A1 inhibitor on the basis of TDP-43 aggregation. In this aspect, the method may include (i) determining that the patient exhibits, or is prone to develop, TDP-43 aggregation, and (ii) providing to the patient a therapeutically effective amount of a CYP51A1 inhibitor. In some embodiments, the patient has previously been determined to exhibit, or to be prone to developing, TDP-43 aggregation, and the method includes providing to the patient a therapeutically effective amount of a CYP51A1 inhibitor. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In an additional aspect, the invention features a method of treating a neurological disorder in a patient, such as a human patient, identified as likely to benefit from treatment with a CYP51A1 inhibitor on the basis of TDP-43 expression. In this aspect, the method includes (i) determining that the patient expresses a mutant form of TDP-43 having a mutation associated with TDP-43 aggregation (e.g., a mutation selected from Q331K, M337V, Q343R, N345K, R361S, and N390D), and (ii) providing to the patient a therapeutically effective amount of a CYP51A1 inhibitor. In some embodiments, the patient has previously been determined to express a mutant form of TDP-43 having a mutation associated with TDP-43 aggregation, such as a Q331K, M337V, Q343R, N345K, R361S, or N390D mutation, and the method includes providing to the patient a therapeutically effective amount of a CYP51A1 inhibitor.

In another aspect, the invention features a method of determining whether a patient (e.g., a human patient) having a neurological disorder is likely to benefit from treatment with a CYP51A1 inhibitor by (i) determining whether the patient exhibits, or is prone to develop, TDP-43 aggregation and (ii) identifying the patient as likely to benefit from treatment with a CYP51A1 inhibitor if the patient exhibits, or is prone to develop, TDP-43 aggregation. In some embodiments, the method further includes the step of (iii) informing the patient whether he or she is likely to benefit from treatment with a CYP51A1 inhibitor. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In another aspect, the invention features a method of determining whether a patient (e.g., a human patient) having a neurological disorder is likely to benefit from treatment with a CYP51A1 inhibitor by (i) determining whether the patient expresses a TDP-43 mutant having a mutation associated with TDP-43 aggregation (e.g., a mutation selected from Q331K, M337V, Q343R, N345K, R361S, and N390D) and (ii) identifying the patient as likely to benefit from treatment with a CYP51 A1 inhibitor if the patient expresses a TDP-43 mutant. In some embodiments, the method further includes the step of (iii) informing the patient whether he or she is likely to benefit from treatment with a CYP51A1 inhibitor. The TDP-43 isoform expressed by the patient may be assessed, for example, by isolated TDP-43 protein from a sample obtained from the patient and sequencing the protein using molecular biology techniques described herein or known in the art. In some embodiments, the TDP-43 isoform expressed by the patient is determined by analyzing the patient’s genotype at the TDP-43 locus, for example, by sequencing the TDP-43 gene in a sample obtained from the patient. In some embodiments, the method includes the step of obtaining the sample from the patient.

In some embodiments of any of the above aspects, the CYP51A1 inhibitor is provided to the patient by administration of the CYP51A1 inhibitor to the patient. In some embodiments, the CYP51A1 inhibitor is provided to the patient by administration of a prodrug that is converted in vivo to the CYP51A1 inhibitor.

In some embodiments of any of the above aspects, the neurological disorder is a neuromuscular disorder, such as a neuromuscular disorder selected from amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac’s Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome. In some embodiments, the neurological disorder is amyotrophic lateral sclerosis.

In some embodiments of any of the above aspects, the neurological disorder is selected from frontotemporal degeneration (also referred to as frontotemporal lobar degeneration and frontotemporal dementia), Alzheimer’s disease, Parkinson’s disease, dementia with Lewy Bodies, corticobasal degeneration, progressive supranuclear palsy, dementia parkinsonism ALS complex of Guam, Huntington’s disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy.

In some embodiments, the neurological disorder is amyotrophic lateral sclerosis, and following administration of the CYP51A1 inhibitor to the patient, the patient exhibits one or more, or all, of the following responses:
(i) an improvement in condition as assessed using the amyotrophic lateral sclerosis functional rating scale (ALSFRS) or the revised ALSFRS (ALSFRS-R), such as an improvement in the patient’s ALSFRS or ALSFRS-R score within one or more days, weeks, or months following administration of the CYP51A1 inhibitor (e.g., an improvement in the patient’s ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the patient);
(ii) an increase in slow vital capacity, such as an increase in the patient’s slow vital capacity within one or more days, weeks, or months following administration of the CYP51A1 inhibitor (e.g., an increase in the patient’s slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the patient);
(iii) a reduction in decremental responses exhibited by the patient upon repetitive nerve stimulation, such as a reduction that is observed within one or more days, weeks, or months following administration of the CYP51A1 inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the CYP51A1 inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51 A1 inhibitor to the patient);
(v) an improvement in quality of life, as assessed, for example, using the amyotrophic lateral sclerosis-specific quality of life (ALS-specific QOL) questionnaire, such as an improvement in the patient’s quality of life that is observed within one or more days, weeks, or months following administration of the CYP51A1 inhibitor (e.g., an improvement in the subject’s quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51 A1 inhibitor to the patient);
(vi) a decrease in the frequency and/or severity of muscle cramps, such as a decrease in cramp frequency and/or severity within one or more days, weeks, or months following administration of the CYP51A1 inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the patient); and/or
(vii) a decrease in TDP-43 aggregation, such as a decrease in TDP-43 aggregation within one or more days, weeks, or months following administration of the CYP51A1 inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51 A1 inhibitor to the patient.

### Chemical Terms

It is to be understood that the terminology employed herein is for the purpose of describing particular embodiments and is not intended to be limiting.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone - enol pairs, amide - imidic acid pairs, lactam - lactim pairs, amide - imidic acid pairs, enamine - imine pairs, and annular forms where a proton can occupy two or more positions of a heterocyclic system, such as, 1H- and 3H-imidazole, 1H-, 2H- and 4H- 1,2,4-triazole, 1H- and 2H- isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

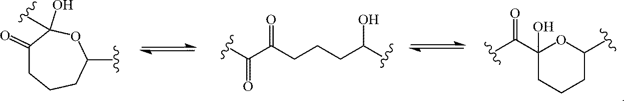

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physiciochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, e.g., in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2} SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-₁₀ aryl, C₁-C₁₀ alkyl C₆-₁₀ aryl, or C₁-C₂₀ alkyl C₆-₁₀ aryl), such as, benzyl and phenethyl. In some embodiments, the akyl and the aryl each can be further substituted with 1, 2, 3, or 4 substituent groups as defined herein for the respective groups.

The term “azido,” as used herein, represents a —N₃ group.

The term “cyano,” as used herein, represents a CN group.

The terms “carbocyclyl,” as used herein, refer to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl—O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl—O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl—O—. A heteroalkynylene is a divalent heteroalkynyl group.

The term “heteroaryl,” as used herein, refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the akyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S and no aromatic ring containing any N, O, or S atoms. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the akyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example, aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained, for example, by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon- carbon double bond may be in an E (substituents are on opposite sides of the carbon- carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “administration” refers to the administration of a composition (e.g., a compound, a complex or a preparation that includes a compound or complex as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population).

As used herein, the terms “benefit” and “response” are used interchangeably in the context of a subject, such as a human subject undergoing therapy for the treatment of a neurological disorder, for example, amyotrophic lateral sclerosis, frontotemporal degeneration (also referred to as frontotemporal lobar degeneration and frontotemporal dementia), Alzheimer’s disease, Parkinson’s disease, dementia with Lewy Bodies, corticobasal degeneration, progressive supranuclear palsy, dementia parkinsonism ALS complex of Guam, Huntington’s disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. The terms “benefit” and “response” refer to any clinical improvement in the subject’s condition. Exemplary benefits in the context of a subject undergoing treatment for a neurological disorder using the compositions and methods described herein (e.g., in the context of a human subject undergoing treatment for a neurological disorder described herein, such as amyotrophic lateral sclerosis, with a cytochrome P450 isoform 51A1 (CYP51A1) inhibitor described herein, such as an inhibitory small molecule, antibody, antigen-binding fragment thereof, or interfering RNA molecule) include the slowing and halting of disease progression, as well as suppression of one or more symptoms associated with the disease. Particularly, in the context of a patient (e.g., a human patient) undergoing treatment for amyotrophic lateral sclerosis with a CYP51A1 inhibitor described herein, examples of clinical “benefits” and “responses” are (i) an improvement in the subject’s condition as assessed using the amyotrophic lateral sclerosis functional rating scale (ALSFRS) or the revised ALSFRS (ALSFRS-R) following administration of the CYP51A1 inhibitor, such as an improvement in the subject’s ALSFRS or ALSFRS-R score within one or more days, weeks, or months following administration of the CYP51A1 inhibitor (e.g., an improvement in the subject’s ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the subject); (ii) an increase in the subject’s slow vital capacity following administration of the CYP51A1 inhibitor, such as an increase in the subject’s slow vital capacity within one or more days, weeks, or months following administration of the CYP51A1 inhibitor (e.g., an increase in the subject’s slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the subject); (iii) a reduction in decremental responses exhibited by the subject upon repetitive nerve stimulation, such as a reduction that is observed within one or more days, weeks, or months following administration of the CYP51A1 inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the subject); (iv) an improvement in the subject’s muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the CYP51A1 inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51 A1 inhibitor to the subject); (v) an improvement in the subject’s quality of life, as assessed, for example, using the amyotrophic lateral sclerosis-specific quality of life (ALS-specific QOL) questionnaire, such as an improvement in the subject’s quality of life that is observed within one or more days, weeks, or months following administration of the CYP51A1 inhibitor (e.g., an improvement in the subject’s quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the subject); and (vi) a decrease in the frequency and/or severity of muscle cramps exhibited by the subject, such as a decrease in cramp frequency and/or severity within one or more days, weeks, or months following administration of the CYP51A1 inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the subject).

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

As used herein, the terms “cytochrome P450 isoform 51A1,” “CYP51A1,” and “lanosterol 14-alpha demethylase” are used interchangeably and refer to the enzyme that catalyzes the conversion of lanosterol to 4,4-dimethylcholesta-8(9),14,24-trien-3β-ol, for example, in human subjects. The terms “cytochrome P450 isoform 51A1,” “CYP51A1,” and “lanosterol 14-alpha demethylase” refer not only to wild-type forms of CYP51A1, but also to variants of wild-type CYP51A1 proteins and nucleic acids encoding the same. The amino acid sequence and corresponding mRNA sequence of a wild-type form of human CYP51A1 are provided herein as SEQ ID NOs: 1 and 2, which correspond to GenBank Accession No. AAC50951.1 and NCBI Reference Sequence NO. NM_000786.3, respectively. These sequences are shown in Table 2, below.

TABLE 2

Amino acid and nucleic acid sequences of wild-type human CYP5A1

| SEQ ID NO. | Sequence |
|---|---|
| 1 | MLLLGLLQAGGSVLGQAMEKVTGGNLLSMLLIACAFTLSLVYLIRLAAGHLVQLPAG VKSPPYIFSPIPFLGHAIAFGKSPIEFLENAYEKYGPVFSFTMVGKTFTYLLGSDAAAL LFNSKNEDLNAEDVYSRLTTPVFGKGVAYDVPNPVFLEQKKMLKSGLNIAHFKQHV SIIEKETKEYFESWGESGEKNVFEALSELIILTASHCLHGKEIRSQLNEKVAQLYADL DGGFSHAAWLLPGWLPLPSFRRRDRAHREIKDIFYKAIQKRRQSQEKIDDILQTLLD ATYKDGRPLTDDEVAGMLIGLLLAGQHTSSTTSAWMGFFLARDKTLQKKCYLEQKT VCGENLPPLTYDQLKDLNLLDRCIKETLRLRPPIMIMMRMARTPQTVAGYTIPPGHQ VCVSPTVNQRLKDSWVERLDFNPDRYLQDNPASGEKFAYVPFGAGRHRCIGENFA YVQIKTIWSTMLRLYEFDLIDGYFPTVNYTTMIHTPENPVIRYKRRSK |
| 2 | ATGGCGGCGGCGGCTGGGATGCTGCTGCTGGGCTTGCTGCAGGCGGGTGGG TCGGTGCTGGGCCAGGCGATGGAGAAGGTGACAGGCGGCAACCTCTTGTCCA TGCTGCTGATCGCCTGCGCCTTCACCCTCAGCCTGGTCTACCTGATCCGTCTG GCCGCCGGCCACCTGGTCCAGCTGCCCGCAGGGGTGAAAAGTCCTCCATACA TTTTCTCCCCAATTCCATTCCTTGGGCATGCCATAGCATTTGGGAAAAGTCCAAT TGAATTTCTAGAAAATGCATATGAGAAGTATGGACCTGTATTTAGTTTTACCATG GTAGGCAAGACATTTACTTACCTTCTGGGGAGTGATGCTGCTGCACTGCTTTTT AATAGTAAAAATGAAGACCTGAATGCAGAAGATGTCTACAGTCGCCTGACAACA CCTGTGTTTGGGAAGGGAGTTGCATACGATGTGCCTAATCCAGTTTTCTTGGAG CAGAAGAAAATGTTAAAAAGTGGCCTTAACATAGCCCACTTTAAACAGCATGTTT CTATAATTGAAAAAGAAACAAAGGAATACTTTGAGAGTTGGGGAGAAAGTGGAG AAAAAAATGTGTTTGAAGCTCTTTCTGAGCTCATAATTTTAACAGCTAGCCATTG TTTGCATGGAAAGGAAATCAGAAGTCAACTCAATGAAAAGGTAGCACAGCTGTA TGCAGATTTGGATGGAGGTTTCAGCCATGCAGCCTGGCTCTTACCAGGTTGGC TGCCTTTGCCTAGTTTCAGACGCAGGGACAGAGCTCATCGGGAAATCAAGGAT ATTTTCTATAAGGCAATCCAGAAACGCAGACAGTCTCAAGAAAAAATTGATGACA TTCTCCAAACTTTACTAGATGCTACATACAAGGATGGGCGTCCTTTGACTG ATGATGAAGTAGCAGGGATGCTTATTGGATTACTCTTGGCAGGGCAGCATACAT CCTCAACTACTAGTGCTTGGATGGGCTTCTTTTTGGCCAGAGACAAAACACTTC AAAAAAAATGTTATTTAGAACAGAAAACAGTCTGTGGAGAGAATCTGCCTCCTTT AACTTATGACCAGCTCAAGGATCTAAATTTACTTGATCGCTGTATAAAAGAAACA TTAAGACTTAGACCTCCTATAATGATCATGATGAGAATGGCCAGAACTCCTCAG ACTGTGGCAGGGTATACCATTCCTCCAGGACATCAGGTGTGTGTTTCTCCCACT GTCAATCAAAGACTTAAAGACTCATGGGTAGAACGCCTGGACTTTAATCCTGAT CGCTACTTACAGGATAACCCAGCATCAGGGGAAAAGTTTGCCTATGTGCCATTT GGAGCTGGGCGTCATCGTTGTATTGGGGAAAATTTTGCCTATGTTCAAATTAAG ACAATTTGGTCCACTATGCTTCGTTTATATGAATTTGATCTCATTGATGGATACTT TCCCACTGTGAATTATACAACTATGATTCACACCCCTGAAAACCCAGTTATCCGT TACAAACGAAGATCAAAATGA |

The terms “cytochrome P450 isoform 51A1,” “CYP51A1,” and “lanosterol 14-alpha demethylase” as used herein include, for example, forms of the human CYP51A1 protein that have an amino acid sequence that is at least 85% identical to the amino acid sequence of SEQ ID NO: 1 (e.g., 85%, 86%, 87%, 88%, 89%, 90%, 91%, 92%, 93%, 94%, 95%, 96%, 97%, 98%, 99%, 99.9%, or 100% identical to the amino acid sequence of SEQ ID NO: 1) and/or forms of the human CYP51A1 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type CYP51A1 protein. Similarly, the terms “cytochrome P450 isoform 51A1,” “CYP51A1,” and “lanosterol 14-alpha demethylase” as used herein include, for example, forms of the human CYP51A1 gene that encode an mRNA transcript having a nucleic acid sequence that is at least 85% identical to the nucleic acid sequence of SEQ ID NO: 2 (e.g., 85%, 86%, 87%, 88%, 89%, 90%, 91%, 92%, 93%, 94%, 95%, 96%, 97%, 98%, 99%, 99.9%, or 100% identical to the amino acid sequence of SEQ ID NO: 2).

As used herein, the terms “cytochrome P450 isoform 51A1 inhibitor,” “CYP51A1 inhibitor,” and “lanosterol 14-alpha demethylase inhibitor” are used interchangeably and refer to substances, such as compounds of Formula I. Inhibitors of this type may, for example, competitively inhibit CYP51A1 activity by specifically binding the CYP51A1 enzyme (e.g., by virtue of the affinity of the inhibitor for the CYP51A1 active site), thereby precluding, hindering, or halting the entry of one or more endogenous substrates of CYP51A1 into the enzyme’s active site. Additional examples of CYP51A1 inhibitors that suppress the activity of the CYP51A1 enzyme include substances that may bind CYP51A1 at a site distal from the active site and attenuate the binding of endogenous substrates to the CYP51A1 active site by way of a change in the enzyme’s spatial conformation upon binding of the inhibitor. In addition to encompassing substances that modulate CYP51A1 activity, the terms “cytochrome P450 isoform 51A1 inhibitor,” “CYP51A1 inhibitor,” and “lanosterol 14-alpha demethylase inhibitor” refer to substances that reduce the concentration and/or stability of CYP51A1 mRNA transcripts in vivo, as well as those that suppress the translation of functional CYP51A1 enzyme.

As used herein, the term “CYP51A1-associated disorder” refers to an undesired physiological condition, disorder, or disease that is associated with and/or mediated at least in part by CYP51A1. In some instances, CYP51A1-associated disorders are associated with excess CYP51A1 levels and/or activity. Exemplary CYP51A1-associated disorders include CYP51A1-associated disorders include but are not limited to central nervous system (CNS) disorders, dementia, Alzheimer’s Disease, chronic traumatic encephalopathy, FTLD-TDP, LATE, or frontotemporal lobar degeneration.

As used herein, the term “combination therapy” refers to those situations in which a subject is simultaneously exposed to two or more therapeutic agents. In some embodiments, two or more compounds may be administered simultaneously; in some embodiments, such compounds may be administered sequentially; in some embodiments, such compounds are administered in overlapping dosing regimens.

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

As used herein, the term “neuromuscular disorder” refers to a disease impairing the ability of one or more neurons to control the activity of an associated muscle. Examples of neuromuscular disorders are amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac’s Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome, among others.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (e.g., a vehicle capable of suspending or dissolving the active compound) and having the properties of being substantially nontoxic and non-inflammatory in a patient. Excipients may include, for example, antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration.

As used herein, the term “pharmaceutically acceptable salt” means any pharmaceutically acceptable salt of the compound of formula (I). For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P.H. Stahl and C.G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The term “pure” means substantially pure or free of unwanted components (e.g., other compounds and/or other components of a cell lysate), material defilement, admixture or imperfection.

A variety of clinical indicators can be used to identify a patient as “at risk” of developing a particular neurological disease. Examples of patients (e.g., human patients) that are “at risk” of developing a neurological disease, such as amyotrophic lateral sclerosis, frontotemporal degeneration, Alzheimer’s disease, Parkinson’s disease, dementia with Lewy Bodies, corticobasal degeneration, progressive supranuclear palsy, dementia parkinsonism ALS complex of Guam, Huntington’s disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include (i) subjects exhibiting or prone to exhibit aggregation of TAR-DNA binding protein (TDP)-43, and (ii) subjects expressing a mutant form of TDP-43 containing a mutation associated with TDP-43 aggregation and toxicity, such as a mutation selected from Q331K, M337V, Q343R, N345K, R361S, and N390D. Subjects that are “at risk” of developing amyotrophic lateral sclerosis may exhibit one or both of these characteristics, for example, prior to the first administration of a CYP51A1 inhibitor in accordance with the compositions and methods described herein.

As used herein, the terms “TAR-DNA binding protein-43” and “TDP-43” are used interchangeably and refer to the transcription repressor protein involved in modulating HIV-1 transcription and alternative splicing of the cystic fibrosis transmembrane conductance regulator (CFTR) pre-mRNA transcript, for example, in human subjects. The terms “TAR-DNA binding protein-43” and “TDP-43” refer not only to wild-type forms of TDP-43, but also to variants of wild-type TDP-43 proteins and nucleic acids encoding the same. The amino acid sequence and corresponding mRNA sequence of a wild-type form of human TDP-43 are provided herein as SEQ ID NOs: 3 and 4, which correspond to NCBI Reference Sequence NOs. NM_007375.3 and NP_031401.1, respectively. These sequences are shown in Table 3, below.

TABLE 3

Amino acid and nucleic acid sequences of wild-type human TDP-43

| SEQ ID NO. | Sequence |
|---|---|
| 3 | MSEYIRVTEDENDEPIEIPSEDDGTVLLSTVTAQFPGACGLRYRNPVSQCMRGVRL VEGILHAPDAGWGNLVYVVNYPKDNKRKMDETDASSAVKVKRAVQKTSDLIVLGLP WKTTEQDLKEYFSTFGEVLMVQVKKDLKTGHSKGFGFVRFTEYETQVKVMSQRH MIDGRWCDCKLPNSKQSQDEPLRSRKVFVGRCTEDMTEDELREFFSQYGDVMDV FIPKPFRAFAFVTFADDQIAQSLCGEDLIIKGISVHISNAEPKHNSNRQLERSGRFGG NPGGFGNQGGFGNSRGGGAGLGNNQGSNMGGGMNFGAFSINPAMMAAAQAAL QSSWGMMGMLASQQNQSGPSGNNQNQGNMQREPNQAFGSGNNSYSGSNSGA AIGWGSASNAGSGSGFNGGFGSSMDSKSSGWGM |
| 4 | ATGTCTGAATATATTCGGGTAACCGAAGATGAGAACGATGAGCCCATTGAAATA CCATCGGAAGACGATGGGACGGTGCTGCTCTCCACGGTTACAGCCCAGTTTCC AGGGGCGTGTGGGCTTCGCTACAGGAATCCAGTGTCTCAGTGTATGAGAGGTG TCCGGCTGGTAGAAGGAATTCTGCATGCCCCAGATGCTGGCTGGGGAAATCTG GTGTATGTTGTCAACTATCCAAAAGATAACAAAAGAAAAATGGATGAGACAGAT GCTTCATCAGCAGTGAAAGTGAAAAGAGCAGTCCAGAAAACATCCGATTTAATA GTGTTGGGTCTCCCATGGAAAACAACCGAACAGGACCTGAAAGAGTATTTTAGT ACCTTTGGAGAAGTTCTTATGGTGCAGGTCAAGAAAGATCTTAAGACTGGTCAT TCAAAGGGGTTTGGCTTTGTTCGTTTTACGGAATATGAAACACAAGTGAAAGTA ATGTCACAGCGACATATGATAGATGGACGATGGTGTGACTGCAAACTTCCTAAT TCTAAGCAAAGCCAAGATGAGCCTTTGAGAAGCAGAAAAGTGTTTGTGGGGCG CTGTACAGAGGACATGACTGAGGATGAGCTGCGGGAGTTCTTCTCTCAGTACG GGGATGTGATGGATGTCTTCATCCCCAAGCCATTCAGGGCCTTTGCCTTTGTTA CATTTGCAGATGATCAGATTGCGCAGTCTCTTTGTGGAGAGGACTTGATCATTA AAGGAATCAGCGTTCATATATCCAATGCCGAACCTAAGCACAATAGCAATAGAC AGTTAGAAAGAAGTGGAAGATTTGGTGGTAATCCAGGTGGCTTTGGGAATCAG GGTGGATTTGGTAATAGCAGAGGGGGTGGAGCTGGTTTGGGAAACAATCAAG GTAGTAATATGGGTGGTGGGATGAACTTTGGTGCGTTCAGCATTAATCCAGCCA TGATGGCTGCCGCCCAGGCAGCACTACAGAGCAGTTGGGGTATGATGGGCAT GTTAGCCAGCCAGCAGAACCAGTCAGGCCCATCGGGTAATAACCAAAACCAAG GCAACATGCAGAGGGAGCCAAACCAGGCCTTCGGTTCTGGAAATAACTCTTATA GTGGCTCTAATTCTGGTGCAGCAATTGGTTGGGGATCAGCATCCAATGCAGGG TCGGGCAGTGGTTTTAATGGAGGCTTTGGCTCAAGCATGGATTCTAAGTCTTCT GGCTGGGGAATGTAG |

The terms “TAR-DNA binding protein-43” and “TDP-43” as used herein include, for example, forms of the human TDP-43 protein that have an amino acid sequence that is at least 85% identical to the amino acid sequence of SEQ ID NO: 3 (e.g., 85%, 86%, 87%, 88%, 89%, 90%, 91%, 92%, 93%, 94%, 95%, 96%, 97%, 98%, 99%, 99.9%, or 100% identical to the amino acid sequence of SEQ ID NO: 3) and/or forms of the human TDP-43 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type TDP-43 protein. For instance, patients that may be treated for a neurological disorder as described herein, such as amyotrophic lateral sclerosis, frontotemporal degeneration, Alzheimer’s disease, Parkinson’s disease, dementia with Lewy Bodies, corticobasal degeneration, progressive supranuclear palsy, dementia parkinsonism ALS complex of Guam, Huntington’s disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include human patients that express a form of TDP-43 having a mutation associated with elevated TDP-43 aggregation and toxicity, such as a mutation selected from Q331K, M337V, Q343R, N345K, R361S, and N390D. Similarly, the terms “TAR-DNA binding protein-43” and “TDP-43” as used herein include, for example, forms of the human TDP-43 gene that encode an mRNA transcript having a nucleic acid sequence that is at least 85% identical to the nucleic acid sequence of SEQ ID NO: 4 (e.g., 85%, 86%, 87%, 88%, 89%, 90%, 91%, 92%, 93%, 94%, 95%, 96%, 97%, 98%, 99%, 99.9%, or 100% identical to the amino acid sequence of SEQ ID NO: 4).

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

A “therapeutic regimen” refers to a dosing regimen whose administration across a relevant population is correlated with a desired or beneficial therapeutic outcome.

The term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### BRIEF DESCRIPTION OF THE DRAWINGS

FIGS. 1A - 1C demonstrate that the viability of a yeast TDP-43 model is restored by the Erg11 inhibitor, fluconazole. (FIG. 1A) Structure of the Erg11 inhibitor and anti-fungal, fluconazole. (FIG. 1B) Fluconazole rescues viability of TDP-43-expressing yeast using a resazurin-reduction endpoint. A 2-fold serial dilution of fluconazole was applied to TDP-43-expressing yeast for 24 hours prior to analysis. (FIG. 1C) Wild-type yeast cultures were treated with fluconazole for eight hours prior to HPLC analysis for lanosterol and ergosterol. Data are expressed as the area under the curve (AUC) normalized to cell mass based on optical density of cultures at 600 nm. Fluconazole treatment reduces ergosterol, while simultaneously leading to an increase in the Erg11 substrate, lanosterol.

FIG. 2 shows the structures of compounds used in primary rat cortical neuron TDP-43 wild type and Q331K mutant survival studies.

FIGS. 3A and 3B demonstrate that compound A promotes survival in primary rat cortical neurons transfected with wild-type TDP-43. Rat primary cortical neurons were co-transfected with a red fluorescent protein (RFP) as a morphological marker and either control (empty vector) or wild-type TDP-43 expression plasmids and treated with vehicle (DMSO) or a titration of compound A. (FIG. 3A) Risk of neuron death plots. The lifetime of each neuron was determined by either loss of RFP signal or morphological indicators of death such as loss of neurites and cell blebbing and used to generate cumulative hazard plots of risk of death over time (hrs) post-transfection. (FIG. 3B) Forest plots. Hazard ratios for each treatment group (relative to TDP-43 DMSO group) were determined by cox regression analysis and used to generate forest plots. Hazard ratios (HR) < 1 in which the confidence interval (CI) does not encompass 1 represent treatments that significantly reduce probability of neuron death relative to the TDP-43 DMSO control. P, p-value.

FIGS. 4A and 4B demonstrate that compound A promotes survival in primary rat cortical neurons transfected with Q331K Mutant TDP-43. Rat primary cortical neurons were co-transfected with a red fluorescent protein (RFP) as a morphological marker and either control (empty vector) or Q331K mutant TDP-43 expression plasmids and treated with vehicle (DMSO) or a titration of compound A. (FIG. 4A) Risk of neuron death plots. The lifetime of each neuron was determined by either loss of RFP signal or morphological indicators of death such as loss of neurites and cell blebbing and used to generate cumulative hazard plots of risk of death over time (hrs) post-transfection. (FIG. 4B) Forest plots. Hazard ratios for each treatment group (relative to TDP-43 DMSO group) were determined by cox regression analysis and used to generate forest plots. Hazard ratios (HR) < 1 in which the confidence interval (CI) does not encompass 1 represent treatments that significantly reduce probability of neuron death relative to the TDP-43 DMSO control. P, p-value.

FIGS. 5A and 5B demonstrate that compound B promotes survival in primary rat cortical neurons transfected with wild-type TDP-43. Rat primary cortical neurons were co-transfected with a red fluorescent protein (RFP) as a morphological marker and either control (empty vector) or wild type TDP-43 expression plasmids and treated with vehicle (DMSO) or a titration of compound B. (FIG. 5A) Risk of neuron death plots. The lifetime of each neuron was determined by either loss of RFP signal or morphological indicators of death such as loss of neurites and cell blebbing and used to generate cumulative hazard plots of risk of death over time (hrs) post-transfection. (FIG. 5B) Forest plots. Hazard ratios for each treatment group (relative to TDP-43 DMSO group) were determined by cox regression analysis and used to generate forest plots. Hazard ratios (HR) < 1 in which the confidence interval (CI) does not encompass 1 represent treatments that significantly reduce probability of neuron death relative to the TDP-43 DMSO control. P, p-value.

### DETAILED DESCRIPTION

The present invention features compositions and methods for treating neurological disorders, such as amyotrophic lateral sclerosis and other neuromuscular disorders, as well as frontotemporal degeneration, Alzheimer’s disease, Parkinson’s disease, dementia with Lewy Bodies, corticobasal degeneration, progressive supranuclear palsy, dementia parkinsonism ALS complex of Guam, Huntington’s disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy among others. Particularly, the invention provides inhibitors of cytochrome P450 isoform 51A1 (CYP51A1), also referred to herein as lanosterol 14-alpha demethylase, that may be administered to a patient (e.g., a human patient) so as to treat or prevent a neurological disorder, such as one or more of the foregoing conditions. In the context of therapeutic treatment, the CYP51 A1 inhibitor may be administered to the patient to alleviate one or more symptoms of the disorder and/or to remedy an underlying molecular pathology associated with the disease, such as to suppress or prevent aggregation of TAR-DNA binding protein (TDP)-43.

The disclosure herein is based, in part, on the discovery that CYP51A1 inhibition modulates TDP-43 aggregation in vivo. Suppression of TDP-43 aggregation exerts beneficial effects in patients suffering from a neurological disorder. Many pathological conditions have been correlated with TDP-43-promoted aggregation and toxicity, such as amyotrophic lateral sclerosis, frontotemporal degeneration, Alzheimer’s disease, Parkinson’s disease, dementia with Lewy Bodies, corticobasal degeneration, progressive supranuclear palsy, dementia parkinsonism ALS complex of Guam, Huntington’s disease, IBMPFD, sporadic inclusion body myositis, myofibrillar myopathy, dementia pugilistica, chronic traumatic encephalopathy, Alexander disease, and hereditary inclusion body myopathy. Without being limited by mechanism, by administering an inhibitor of CYP51A1, patients suffering from diseases associated with TDP-43 aggregation and toxicity may be treated, for example, due to the suppression of TDP-43 aggregation induced by the CYP51A1 inhibitor.

Patients that are likely to respond to CYP51A1 inhibition as described herein include those that have or are at risk of developing TDP-43 aggregation, such as those that express a mutant form of TDP-43 associated with TDP-43 aggregation and toxicity in vivo. Examples of such mutations in TDP-43 that have been correlated with elevated TDP-43 aggregation and toxicity include Q331K, M337V, Q343R, N345K, R361S, and N390D, among others. The compositions and methods described herein thus provide the additional clinical benefit of enabling the identification of patients that are likely to respond to CYP51 A1 inhibitor therapy, as well as processes for treating these patients accordingly.

The sections that follow provide a description of exemplary CYP51A1 inhibitors that may be used in conjunction with the compositions and methods disclosed herein. The sections below additionally provide a description of various exemplary routes of administration and pharmaceutical compositions that may be used for delivery of these substances for the treatment of a neurological disorder.

### CYP51A1Inhibitors

Exemplary CYP51A1 inhibitors described herein include compounds having a structure according to Formula I:

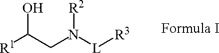

wherein R¹ has the structure:

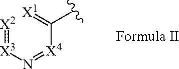

X¹, _{X}², X³, and X⁴ are, independently, N or CR⁴, wherein at least three of X¹, _{X}², X³, and X⁴ are CR⁴;
R² is optionally substituted C₁-C₆ alkyl, optionally substituted C₁-C₆ alkoxy, optionally substituted C₃-C₈ cycloalkyl, optionally substituted C₂-C₉ heterocylcyl, optionally substituted C₂-C₉ heteroaryl, optionally substituted C₁-C₆ alkyl C₃-C₈ cycloalkyl, optionally substituted C₁-C₆ alkyl C₂-C₉ heterocylcyl, optionally substituted C₁-C₆ alkyl C₂-C₉ heteroaryl, or optionally substituted C₁-C₆ alkyl C₆-C₁₀ aryl;
R³ is optionally substituted C₁-C₆ alkyl, optionally substituted C₁-C₆ alkoxy, optionally substituted C₃-C₈ cycloalkyl, optionally substituted C₂-C₉ heterocylcyl, optionally substituted C₂-C₉ heteroaryl, optionally substituted C₁-C₆ alkyl C₃-C₈ cycloalkyl, optionally substituted C₁-C₆ alkyl C₂-C₉ heterocylcyl, optionally substituted C₁-C₆ alkyl C₂-C₉ heteroaryl, or optionally substituted C₁-C₆ alkyl C₆-C₁₀ aryl, wherein if R² is methyl then R³ is optionally substituted C₁-C₆ alkyl, optionally substituted C₁-C₆ alkoxy, optionally substituted C₃-C₈ cycloalkyl, or optionally substituted C₁-C₆ alkyl C₃-C₈ cycloalkyl;
each R⁴ is, independently, hydrogen, hydroxy, halo, cyano, optionally substituted amino, optionally substituted C₁-C₆ alkyl, or optionally substituted C₁-C₆ alkoxy; and
L is —C(O)— or —SO₂—.

In some embodiments, the compound has the structure of any one of compounds 1-147 in Table 1.

Other embodiments, as well as exemplary methods for the synthesis or production of these compounds, are described herein.

### Methods of Treatment

### Suppression of CYP51A1 Activity and TDP-43 Aggregation to Treat Neurological Disorders

Using the compositions and methods described herein, a patient suffering from a neurological disorder may be administered a CYP51A1 inhibitor, such as a small molecule, antibody, antigen-binding fragment thereof, or interfering RNA molecule described herein, so as to treat the disorder and/or to suppress one or more symptoms associated with the disorder. Exemplary neurological disorders that may be treated using the compositions and methods described herein are, without limitation, amyotrophic lateral sclerosis, frontotemporal degeneration, Alzheimer’s disease, Parkinson’s disease, dementia with Lewy Bodies, corticobasal degeneration, progressive supranuclear palsy, dementia parkinsonism ALS complex of Guam, Huntington’s disease, IBMPFD, sporadic inclusion body myositis, myofibrillar myopathy, dementia pugilistica, chronic traumatic encephalopathy, Alexander disease, and hereditary inclusion body myopathy, as well as neuromuscular diseases such as congenital myasthenic syndrome, congenital myopathy, cramp fasciculation syndrome, Duchenne muscular dystrophy, glycogen storage disease type II, hereditary spastic paraplegia, inclusion body myositis, Isaac’s Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome.

The present disclosure is based, in part, on the discovery that CYP51A1 inhibitors, such as the agents described herein, are capable of attenuating TDP-43 aggregation in vivo. TDP-43-promoted aggregation and toxicity have been associated with various neurological diseases. The discovery that CYP51A1 inhibitors modulate TDP-43 aggregation provides an important therapeutic benefit. Using a CYP51A1 inhibitor, such as a CYP51A1 inhibitor described herein, a patient suffering from a neurological disorder or at risk of developing such a condition may be treated in a manner that remedies an underlying molecular etiology of the disease. Without being limited by mechanism, the compositions and methods described herein can be used to treat or prevent such neurological conditions, for example, by suppressing the TDP-43 aggregation that promotes pathology.

Additionally, the compositions and methods described herein provide the beneficial feature of enabling the identification and treatment of patients that are likely to respond to CYP51A1 inhibitor therapy. For example, in some embodiments, a patient (e.g., a human patient suffering from or at risk of developing a neurological disease described herein, such as amyotrophic lateral sclerosis) is administered a CYP51A1 inhibitor if the patient is identified as likely to respond to this form of treatment. Patients may be identified as such on the basis, for example, of susceptibility to TDP-43 aggregation. In some embodiments, the patient is identified is likely to respond to CYP51 A1 inhibitor treatment based on the isoform of TDP-43 expressed by the patient. For example, patients expressing TDP-43 isoforms having a mutation selected from Q331K, M337V, Q343R, N345K, R361S, and N390D, among others, are more likely to develop TDP-43-promoted aggregation and toxicity relative to patients that do not express such isoforms of TDP-43. Using the compositions and methods described herein, a patient may be identified as likely to respond to CYP51A1 inhibitor therapy on the basis of expressing such an isoform of TDP-43, and may subsequently be administered a CYP51A1 inhibitor so as to treat or prevent one or more neurological disorders, such as one or more of the neurological disorders described herein.

### Assessing Patient Response

A variety of methods known in the art and described herein can be used to determine whether a patient having a neurological disorder (e.g., a patient at risk of developing TDP-43 aggregation, such as a patient expressing a mutant form of TDP-43 having a mutation associated with elevated TDP-43 aggregation and toxicity, for example, a mutation selected from Q331K, M337V, Q343R, N345K, R361S, and N390D) is responding favorably to CYP51A1 inhibition. For example, successful treatment of a patient having a neurological disease, such as amyotrophic lateral sclerosis, with a CYP51A1 inhibitor described herein may be signaled by:
(i) an improvement in condition as assessed using the amyotrophic lateral sclerosis functional rating scale (ALSFRS) or the revised ALSFRS (ALSFRS-R), such as an improvement in the patient’s ALSFRS or ALSFRS-R score within one or more days, weeks, or months following administration of the CYP51A1 inhibitor (e.g., an improvement in the patient’s ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the patient);
(ii) an increase in slow vital capacity, such as an increase in the patient’s slow vital capacity within one or more days, weeks, or months following administration of the CYP51A1 inhibitor (e.g., an increase in the patient’s slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the patient);
(iii) a reduction in decremental responses exhibited by the patient upon repetitive nerve stimulation, such as a reduction that is observed within one or more days, weeks, or months following administration of the CYP51A1 inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the CYP51A1 inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51 A1 inhibitor to the patient);
(v) an improvement in quality of life, as assessed, for example, using the amyotrophic lateral sclerosis-specific quality of life (ALS-specific QOL) questionnaire, such as an improvement in the patient’s quality of life that is observed within one or more days, weeks, or months following administration of the CYP51A1 inhibitor (e.g., an improvement in the subject’s quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51 A1 inhibitor to the patient);
(vi) a decrease in the frequency and/or severity of muscle cramps, such as a decrease in cramp frequency and/or severity within one or more days, weeks, or months following administration of the CYP51A1 inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51A1 inhibitor to the patient); and/or
(vii) a decrease in TDP-43 aggregation, such as a decrease in TDP-43 aggregation within one or more days, weeks, or months following administration of the CYP51A1 inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the CYP51A1 inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the CYP51 A1 inhibitor to the patient.

### Combination Formulations and Uses Thereof

The compounds of the invention can be combined with one or more therapeutic agents. In particular, the therapeutic agent can be one that treats or prophylactically treats any neurological disorder described herein.

### Combination Therapies

A compound of the invention can be used alone or in combination with other agents that treat neurological disorders or symptoms associated therewith, or in combination with other types of treatment to treat, prevent, and/or reduce the risk of any neurological disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington’s Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-50 mg/kg.

### EXAMPLES

### List of Abbreviations

NaBH₄ = sodium borohydride
EtOH = ethanol
MeOH = methanol
NEt₃ = triethylamine
DCM = dichloromethane
NaHB(OAc)₃ = sodium triacetoxyborohydride
AcOH = acetic acid
HCI = hydrochloric acid
DMF = N,N-dimethylformamide
THF = tetrahydrofuran
T3P = propylphosphonic anhydride
DIPEA = N,N-diisopropylethylamine
Pyr = pyridine
TFA = trifluoroacetic acid
Ti(O—iPr)₄ = titanium(IV) isopropoxide
HATU = N-[(dimethylamino)-1H-1,2,3-triazolo-[4,5-b]pyridin-1-ylmethylene]—N—methylmethanaminium hexafluorophosphate N-oxide
HBTU = N,N,N′,N′-tetramethyl-O-(1H-benzotriazol-1-yl)uronium hexafluorophosphate
DMAP = 4-(dimethylamino)pyridine
KSCN = potassium thiocyanate
Br₂ = bromine
NaCNBH₃ = sodium cyanoboroydride
NaH = sodium hydride
Boc₂O = di(tert-butyl) carbonate
HOBT = 1-hydroxybenzotriazole
DCC = 1,3-dicyclohexylcarbodiimide
EDC = 1-ethyl-3-(3-dimethylaminopropyl)carbodiimide hydrochloride
LiOH x H₂O = lithium hydroxide hydrate

### Example 1. General Schemes

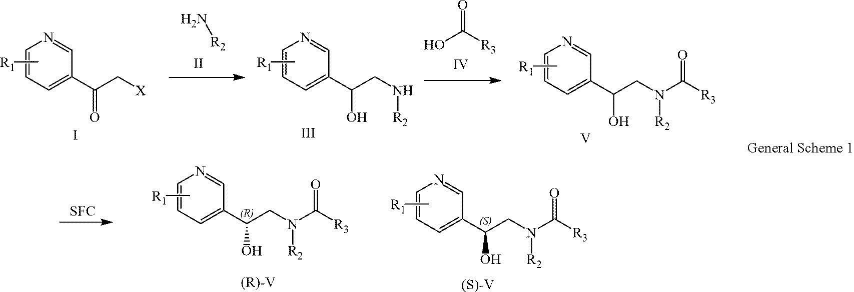

An appropriately substituted pyridine ketone I is reacted with an appropriately substituted amine II in the presence of a reducing agent (e.g. sodium borohydride) to afford the appropriately substituted amine III. Coupling of amine III with acid IV under a variety of coupling conditions (e.g. HOBt/EDC) yields amide V. SFC separation affords two enantiomeric compounds, (S)-V and R-(V).

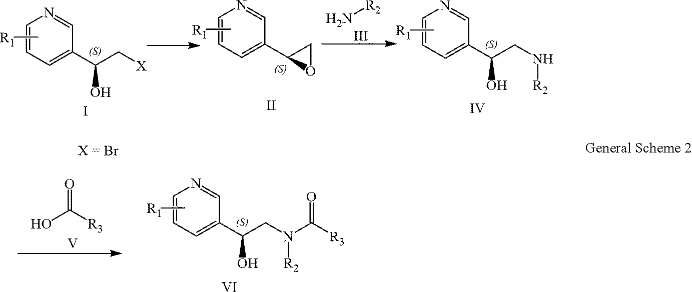

An intramolecular SN2 reaction of appropriately substituted chiral alcohol I under basic conditions affords epoxide II. Opening of epoxide II with appropriately substituted amine III affords B-amino alcohol IV. Coupling of amine IV with appropriately substituted acid V under a variety of coupling conditions (e.g. HOBt/EDC) affords chiral amide VI.

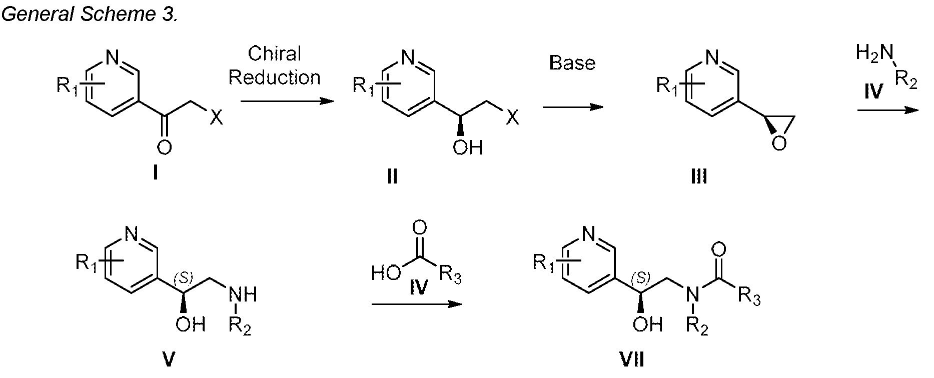 [SMILES: CCCN(CC(O)c1cccnc1)C(=O)Cc1ccc2ccccc2c1]

An appropriately substituted pyridine ketone I is reduced to an alcohol II with a chiral reducing agent (e.g. DIP-CI). Intramolecular SN2 displacement of alkyl halide II under basic condition (e.g. potassium carbonate) affords epoxide III. Opening of epoxide III with appropriately substituted amine IV affords B-amino alcohol V. Coupling of amine V with appropriately substituted acid VI under a variety of coupling conditions (e.g. HBTU) affords chiral amide VII.

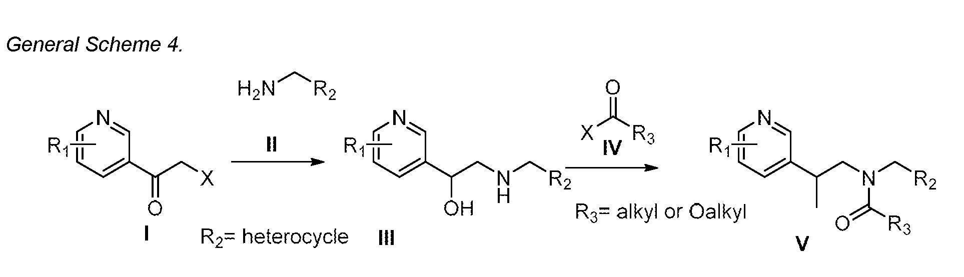 [SMILES: CCC(=O)N(CC(O)c1cccnc1)C(=O)Cc1ccc2ccccc2c1]

An appropriately substituted pyridine ketone I is reacted with an appropriately substituted amine II in the presence of a reducing agent (e.g. sodium borohydride) to afford the appropriately substituted amine III. Acylation of amine III with appropriately substituted acyl chloride or chloroformate IV affords appropriately amide or carbamate V.

### Example 2: Preparation of 2-(3,4-Dichlorophenyl)-N-(2-Hydroxy-2-(None-3-yl)Ethyl)-N-Propylacetamide (Compound 1)

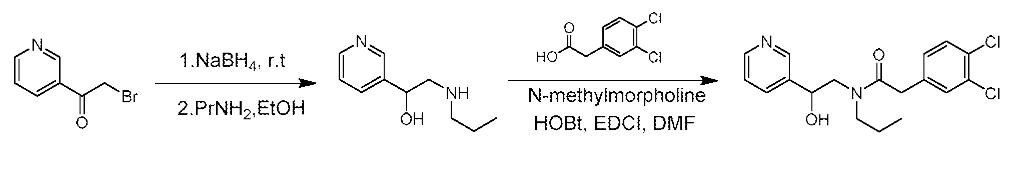 [SMILES: CCC(=O)N(CCc1ccc(Cl)c(Cl)c1)CC(O)c1cccnc1] l

### Step 1: Preparation of 2-(propylamino)-1-(3-pyridyl)ethanol

To a solution of 2-bromo-1-(pyridin-3-yl)ethan-1-one.HBr (3 g, 10.68 mmol) in ethanol (50 mL) was added sodium borohydride (1.62 g, 42.71 mmol). The reaction mixture was stirred at 20° C. for 2 h. The mixture was filtered, and propan-1-amine (1.58 g, 26.70 mmol) was added to the filtrate and the resultant solution was heated to 90° C. and stirred for 4 h. Then ethanol was removed by distillation, the resulting pale yellow solid was dissolved in chloroform (40 mL), the insoluble material was filtered off, and the filtrate was concentrated under reduced pressure. The crude product was purified by flash column (ISCO 20 g silica, 0-30% (methanol : ammonium hydroxide 100:1) in ethyl acetate, gradient over 20 min) to yield 2-(propylamino)-1-(3-pyridyl)ethanol (920 mg, 5.10 mmol, 48%) as a yellow oil. ¹H NMR (400 MHz, Chloroform-d) δ 8.62 (d, J = 1.5 Hz, 1H), 8.53 (dd, J = 1.3, 4.9 Hz, 1H), 7.75 (br. d, J = 7.9 Hz, 1H), 7.31 - 7.27 (m, 1H), 4.86 (dd, J = 3.3, 9.5 Hz, 1H), 3.41 (br. s, 2H), 3.00 (dd, J = 3.5, 12.1 Hz, 1H), 2.80 -2.65 (m, 3H), 1.59 (sxt, J = 7.4 Hz, 2H), 0.96 (t, J = 7.4 Hz, 3H); LCMS (ESI) m/z: 181.1 [M+H]⁺.

### Step 2: Preparation of 2-(3,4-Dichlorophenyl)-N-[2-Hydroxy-2-(3-Pyridyl)ethyl]-N-Propyl-Acetamide

To a solution of 2-(propylamino)-1-(3-pyridyl)ethanol (820 mg, 4.55 mmol) in dimethylformamide (10 mL) were added 2-(3,4-dichlorophenyl)acetic acid (933 mg, 4.55 mmol), 1-hydroxybenzotriazole (615 mg, 4.55 mmol), N-methylmorpholine (920 mg, 9.10 mmol, 1.00 mL) and N-(3-dimethylaminopropyl)-N′-ethylcarbodiimide (959 mg, 5.00 mmol). The mixture was stirred at 20° C. for 16 h. Water (10 mL) was added, and the reaction mixture was extracted with ethyl acetate (20 mL × 2). The combined organic layers were washed with brine (10 mL), dried over sodium sulfate and concentrated. The crude product was purified by flash column (ISCO 20 g silica, 0-10% methanol in ethyl acetate, gradient over 20 min) to give 2-(3,4-dichlorophenyl)-N-[2-hydroxy-2-(3-pyridyl)ethyl]-N-propyl-acetamide (1.31 g, 3.57 mmol, 78%) as a pale yellow oil. ¹H NMR (400 MHz, Chloroform-d) δ 8.58 (d, J = 1.8 Hz, 1H), 8.52 (dd, J = 1.5, 4.9 Hz, 1H), 7.73 (br. d, J = 7.9 Hz, 1H), 7.42 (d, J = 8.4 Hz, 1H), 7.36 (d, J = 2.0 Hz, 1H), 7.32 = 7.27 (m, 1H), 7.11 (dd, J = 2.0, 8.4 Hz, 1H), 5.03 (br. d, J = 7.1 Hz, 1H), 4.75 (br. s, 1H), 3.77 - 3.67 (m, 3H), 3.52 (dd, J = 2.6, 14.3 Hz, 1H), 3.29 - 3.06 (m, 2H), 1.64 - 1.50 (m, 2H), 0.91 (t, J = 7.4 Hz, 3H); LCMS (ESI) m/z: 367.0 [M+H]⁺.

The following compounds were synthesized according to the protocol described for Compound 1:

| Name | Structure | NMR, MS | Compd# |
|---|---|---|---|
| N-[2-hydroxy-2-(3-pyridyl)ethyl]-2-(2-naphthyl)-N-propyl-acetamide | 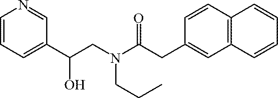 | ¹H NMR (400 MHz, Dimethylsulfoxide-d6, T=273⁺80K) δ 8.69 - 8.35 (m, 2H), 7.95 - 7.57 (m, 5H), 7.54 - 7.19 (m, 4H), 5.74 - 5.30 (m, 1H), 4.95 - 4.83 (m, 1H), 3.84 (s, 2H), 3.74 - 3.31 (m, 3H), 3.23 (td, J = 7.4, 14.3 Hz, 1H), 1.52 (m, 2H), 0.81 (t, J = 7.2 Hz, 3H); LCMS (ESI) m/z: 349.2 [M+H]⁺. | 2 |
| Preparation of N-[2-hydroxy-2-(3-pyridyl)ethyl]-N-[2-(2-naphthyl)ethyl]prope namide | 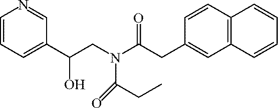 | ¹H NMR (400 MHz, Dimethylsulfoxide-d₆) δ 8.62 - 8.50 (m, 2H), 7.90 - 7.82 (m, 3H), 7.81 - 7.68 (m, 2H), 7.52 -7.32 (m, 4H), 4.90 - 4.83 (m, 1H), 3.67 - 3.44 (m, 3H), 3.29 (m, 1H), 3.04 -2.84 (m, 2H), 2.44 - 2.11 (m, 2H), 0.95 - 0.82 (m, 3H); LCMS (ESI) m/z: 349.2 [M+H]⁺. | 3 |
| N-[2-(3,4-dichlorophenyl)ethyl] -N-[2-hydroxy-2-(3-pyridyl)ethyl]propen amide | 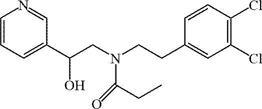 | ¹H NMR (400 MHz, Dimethylsulfoxide-d₆) δ 8.59 (d, J = 1.8 Hz, 0.6H), 8.52 (d, J = 1.8 Hz, 0.4H), 8.47 (ddd, J = 1.5, 4.7, 9.2 Hz, 1H), 7.79 (br. d, J = 7.7 Hz, 0.6H), 7.74 (br. d, J = 7.7 Hz, 0.4H), 7.58 - 7.49 (m, 2H), 7.37 (dt, J = 5.0, 7.4 Hz, 1H), 7.23 (ddd, J = 1.8, 8.3, 12.0 Hz, 1H), 5.76 (d, J = 4.4 Hz, 0.6H), 5.61 (d, J = 4.6 Hz, 0.4H), 4.84 (br. dd, J = 3.9, 7.8 Hz, 1H), 3.65 -3.37 (m, 3H), 3.32 - 3.24 (m, 1H), 2.86 - 2.71 (m, 2H), 2.42 - 2.34 (m, 0.5H), 2.28 - 2.12 (m, 1.5H), 0.92 - 0.86 (m, 3H); LCMS (ESI) m/z: 367.1 [M+H]⁺. | 4 |
| 2-(3,4-dimethoxyphenyl)-N-[2-hydroxy-2-(3-pyridyl)ethyl]-N-propyl-acetamide | 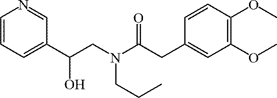 | ¹H NMR (400 MHz, Chloroform-d) δ 8.61 - 8.48 (m, 2H), 7.73 (m, J = 1.6, 7.8 Hz, 1H), 7.25 (m, J = 4.8, 7.8 Hz, 1H), 6.91 - 6.73 (m, 3H), 5.14 (br. s, 1H), 5.00 (dd, J = 1.8, 8.0 Hz, 1H), 3.89 - 3.80(m, 5H), 3.79 - 3.74 (m, 1H), 3.70 (s, 2H), 3.45 (dd, J = 2.4, 14.4 Hz, 1H), 3.29 - 3.16 (m, 1H), 3.07 (m, J = 6.2, 9.2, 15.0 Hz, 1H), 1.63 -1.43 (m, 2H), 0.87 (t, J = 7.2 Hz, 3H); LCMS (ESI) m/z: 359.1 [M+H]⁺. | 5 |
| 2-(3,4-dichlorophenyl)-N-[2-hydroxy-2-(3-pyridyl)ethyl]-N-(1-methylcyclopropyl)a cetamide | 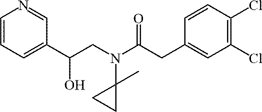 [SMILES: COC(=O)N(CCc1ccc(Cl)c(Cl)c1)CC(O)c1cccnc1] | ¹H NMR (400 MHz, Dimethylsulfoxide-d₆, T=273⁺80k) δ 8.53 (br. s, 1H), 8.46 (br. d, J = 3.7 Hz, 1H), 7.72 (br. d, J = 7.5 Hz, 1H), 7.55 - 7.50 (m, 2H), 7.33 (dd, J = 5.0, 7.6 Hz, 1H), 7.25 (br. d, J = 8.8 Hz, 1H), 5.38 (br. s, 1H), 4.94 (br. s, 1H), 3.93 (br. s, 2H), 3.58 - 3.35 (m, 2H), 1.33 (s, 3H), 1.28 - 1.10 (m, 1H), 0.77 (br. s, 3H); LCMS (ESI) m/z: 379.0 [M+H]⁺. | 9 |
| 2-(3,4-dichlorophenyl)-N-(2-hydroxy-2-methylpropyl)-N-[2-hydroxy-2-(3-pyridyl)ethyl]acetami de | 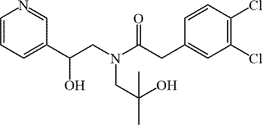 [SMILES: Cc1ccncc1C(O)CN(CC1COC1)C(=O)Cc1ccc(C(F)(F)F)nc1] | ¹H NMR (400 MHz, Chloroform-d) δ 8.56 - 8.40 (m, 2H), 7.80 - 7.71 (m, 1H), 7.43 - 7.32 (m, 2H), 7.28 - 7.23 (m, 1H), 7.08 (dd, J = 2.0, 8.2 Hz, 1H), 5.31 (dd, J = 2.0, 9.0 Hz, 0.6H), 5.00 (dd, J = 3.0, 9.4 Hz, 0.4H), 3.94 - 3.41 (m, 5H), 3.35 - 3.16 (m, 1H), 1.38 - | 43 |
| | | 1.17 (m, 6H); LCMS (ESI) m/z: 397.1 [M+H]⁺. | |
| Methyl N-[2-hydroxy-2-(3-pyridyl)ethyl]-N-[2-(2-naphthyl)ethyl]carba mate | 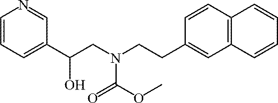 | ¹H NMR (400 MHz, Chloroform-d) δ 8.59 - 8.49 (m, 2H), 7.85 - 7.76 (m, 3H), 7.74 - 7.56 (m, 2H), 7.51 - 7.41 (m, 2H), 7.27 (s, 2H), 4.94 (br. s, 1H), 4.25 (br. s, 1H), 3.72 (s, 3H), 3.66 -3.32 (m, 4H), 2.95 (br. s, 2H). LCMS (ESI) m/z: 351.2 [M+H]⁺. | 82 |
| Methyl N-[2-(3,4-dichlorophenyl)ethyl] -N-[2-hydroxy-2-(3-pyridyl)ethyl]carbam ate | 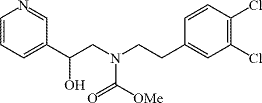 [SMILES: CCCN(CC(O)c1cccnc1)C(=O)Cc1ccc(F)c(F)c1] | ¹H NMR (400 MHz, Dimethylsulfoxide-d₆) δ = 8.55 - 8.49 (m, 1H), 8.49 - 8.40 (m, 1H), 7.75 - 7.68 (m, 1H), 7.53 -7.47 (m, 1H), 7.46 - 7.41 (m, 1H), 7.37 - 7.30 (m, 1H), 7.21 - 7.14 (m, 1H), 5.46 - 5.38 (m, 1H), 4.80 (s, 1H), 3.49 (s, 3H), 3.46 - 3.32 (m, 4H), 2.87 - 2.75 (m, 2H); LCMS (ESI) m/z: 369.0 [M+H]⁺. | 83 |
| N-[2-hydroxy-2-(4-methyl-3-pyridyl)ethyl] -N-(oxetan-3-ylmethyl)-2-[6-(trifluoromethyl)-3-pyridyl]acetamide | 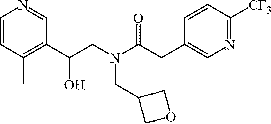 | ¹H NMR (400 MHz, Chloroform-d) δ 8.72 - 8.59 (m, 1H), 8.57 - 8.46 (m, 1H), 8.40 - 8.25 (m, 1H), 7.77 (br. t, J = 8.1 Hz, 1H), 7.69 - 7.58 (m, 1H), 7.12 -7.00 (m, 1H), 5.27 (dd, J = 1.9, 8.9 Hz, 0.5H), 5.14 (br. dd, J = 2.4, 9.1 Hz, 0.5H), 4.87 - 4.80 (m, 1H), 4.76 - 4.66 (m, 1H), 4.51 - 4.35 (m, 2H), 4.05 -3.67 (m, 5H), 3.49 - 3.08 (m, 2H), 2.38 (s, 3H); LCMS (ESI) m/z: 410.2 [M+H]⁺ | 84 |
| N-[2-hydroxy-2-(4-isopropyl-3-pyridyl)ethyl]-N-(oxetan-3-ylmethyl)-2-[6-(trifluoromethyl)-3-pyridyl]acetamide | 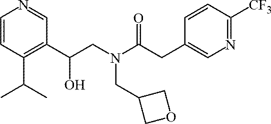 | ¹H NMR (400 MHz, Chloroform-d) δ 8.77 - 8.63 (m, 1H), 8.63 - 8.50 (m, 1H), 8.49 - 8.37 (m, 1H), 7.87 - 7.76 (m, 1H), 7.73 - 7.60 (m, 1H), 7.20 (dd, J = 5.3, 18.1 Hz, 1H), 5.44 - 5.20 (m, 1H), 4.84 (q, J = 6.6 Hz, 1H), 4.78 -4.70 (m, 1H), 4.54 - 4.37 (m, 2H), 4.21 - 3.66 (m, 5H), 3.60 (br. s, 1H), 3.49 -3.08 (m, 3H), 1.34 - 1.03 (m, 6H); LCMS (ESI) m/z: 438.2 [M+H]⁺ | 90 |
| 2-(3,4-difluorophenyl)-N-(2-hydroxy-2-(pyridin-3- | 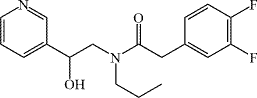 [SMILES: CCCN(CC(O)c1cnccc1OC)C(=O)Cc1ccc(Cl)c(Cl)c1] | ¹H NMR (400 MHz, DMSO-d6) δ = 8.62 (m, J=1.8 Hz, 1H), 8.53 - 8.40 (m, 1H), 7.87 - 7.66 (m, 1H), 7.42 - 7.36 (m, 1H), 7.36 - 7.28 (m, 1H), 7.27 - 7.12 | 95 |
| yl)ethyl)-N-propylacetamide | | (m, 1H), 7.06 - 6.93 (m, 1H), 5.91 -5.55 (m, 1H), 4.91 - 4.79 (m, 1H), 3.78 - 3.61 (m, 2H), 3.61 - 3.46 (m, 1H), 3.44 - 3.34 (m, 2H), 3.24 - 3.10 (m, 1H), 1.55 - 1.42 (m, 2H), 0.84 - 0.74 (m, 3H); LCMS (ESI) m/z: 335.2 [M+H]+ | |
| 2-(3,4-dichlorophenyl)-N-[2-hydroxy-2-(6-methoxy-3-pyridyl)ethyl]-N-propyl-acetamide | 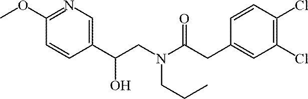 | ¹H NMR (400 MHz, Chloroform-d) δ 8.16 - 8.08 (m, 1H), 7.64 - 7.58 (m, 1H), 7.45 - 7.39 (m, 1H), 7.37 (d, J = 1.7 Hz, 1H), 7.06 (br. d, J = 8.2 Hz, 1H), 6.79 (br. d, J = 8.6 Hz, 1H), 5.04 -4.82 (m, 1H), 4.50 - 4.32 (m, 1H), 4.01 - 3.87 (m, 3H), 3.69 (m, 3H), 3.45 (dd, J = 2.6, 14.2 Hz, 1H), 3.35 - 3.07 (m, 2H), 1.60 - 1.54 (m, 2H), 1.01 - 0.82 (m, 3H); LCMS (ESI) m/z: 397.0 [M+H]⁺ | 102 |
| N-[2-(5-bromo-3-pyridyl)-2-hydroxyethyl]-2-(3,4-dichlorophenyl)-N-propyl-acetamide | 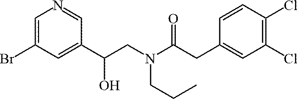 | ¹H NMR (400 MHz, Dimethylsulfoxide-d₆) δ 8.69 - 8.44 (m, 2H), 8.12 - 7.87 (m, 1H), 7.49 (d, J = 8.3 Hz, 1H), 7.43 (br. s, 1H), 7.17 (dd, J = 1.5, 8.3 Hz, 1H), 5.85 - 5.47 (m, 1H), 4.95 - 4.85 (m, 1H), 3.88 - 3.54 (m, 3H), 3.52 -3.16 (m, 3H), 1.76 - 1.41 (m, 2H), 0.84 (br. s, 3H); LCMS (ESI) m/z: 447.0 [M+H]⁺ | 103 |
| 2-(3,4-dichlorophenyl)-N-[2-hydroxy-2-(4-methoxy-3-pyridyl)ethyl]-N-propyl-acetamide | 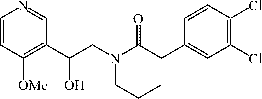 | ¹H NMR (400 MHz, Chloroform-d) δ 8.65 - 8.59 (m, 1H), 8.51 - 8.43 (m, 1H), 7.41 (d, J = 8.2 Hz, 1H), 7.38 -7.31 (m, 1H), 7.13 - 7.06 (m, 1H), 6.84 - 6.76 (m, 1H), 5.24 - 5.12 (m, 1H), 5.03 - 4.81 (m, 1H), 3.96 - 3.76 (m, 4H), 3.73 - 3.53 (m, 2H), 3.43 (br. d, J = 3.0 Hz, 1H), 3.35 - 3.20 (m, 1H), 3.18 - 3.06 (m, 1H), 1.66 - 1.48 (m, 2H), 0.99 - 0.83 (m, 3H); LCMS (ESI) m/z: 397.0 [M+H]⁺ | 109 |
| N-[2-(5-cyano-3-pyridyl)-2-hydroxyethyl]-2-(3,4- | 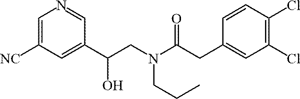 [SMILES: CCCN(CC(O)c1ccc(=O)[nH]c1)C(=O)Cc1ccc(Cl)c(Cl)c1] | ¹H NMR (400 MHz, Dimethylsulfoxide-d₆) δ 8.98 - 8.92 (m, 1H), 8.91 - 8.73 (m, 1H), 8.44 - 8.15 (m, 1H), 7.53 (dd, J = 5.2, 8.3 Hz, 1H), 7.43 (br. d, J = 7.9 | 111 |
| dichlorophenyl)-N-propyl-acetamide | | Hz, 1H), 7.15 (br. dd, J = 8.3, 19.5 Hz, 1H), 6.21 - 5.69 (m, 1H), 5.10 - 4.84 (m, 1H), 3.89 - 3.68 (m, 2H), 3.67 -3.52 (m, 1H), 3.50 - 3.35 (m, 2H), 3.30 - 3.11 (m, 1H), 1.62 - 1.38 (m, 2H), 0.96 - 0.60 (m, 3H); LCMS (ESI) m/z: 392.1 [M+H]⁺ | |
| N-[2-(5-fluoro-3-pyridyl)-2-hydroxyethyl]-N-propyl-2-[6-(trifluoromethyl)-3-pyridyl]acetamide | 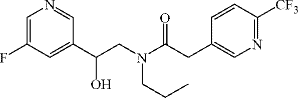 | ¹H NMR (400 MHz, Chloroform-d) δ 8.64 - 8.53 (m, 1H), 8.49 - 8.35 (m, 2H), 7.89 - 7.78 (m, 1H), 7.74 - 7.62 (m, 1H), 7.57 - 7.47 (m, 1H), 5.12 -4.98 (m, 1H), 4.76 - 4.49 (m, 1H), 3.82 (s, 2H), 3.76 - 3.66 (m, 1H), 3.59 - 3.46 (m, 1H), 3.42 - 3.17 (m, 2H), 1.66 -1.55 (m, 2H), 1.04 - 0.85 (m, 3H); LCMS (ESI) m/z: 386.0 | 112 |
| 2-(3,4-dichlorophenyl)-N-[2-hydroxy-2-[6-(trifluoromethyl)-3-pyridyl]ethyl]-N-propyl-acetamide | 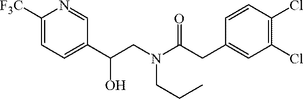 | ¹H NMR (400 MHz, Chloroform-d) δ 8.76 - 8.65 (m, 1H), 7.99 - 7.88 (m, 1H), 7.72 - 7.63 (m, 1H), 7.48 - 7.40 (m, 1H), 7.38 - 7.33 (m, 2H), 7.14 -7.05 (m, 1H), 5.17 - 5.05 (m, 1H), 4.95 - 4.84 (m, 1H), 3.83 - 3.63 (m, 3H), 3.58 - 3.46 (m, 1H), 3.38 - 3.06 (m, 2H), 1.59 - 1.57 (m, 2H), 0.93 (br. t, J = 7.4 Hz, 3H); LCMS (ESI) m/z: 434.9 [M+H]⁺ | 114 |
| 2-(3,4-dichlorophenyl)-N-[2-hydroxy-2-(6-oxo-1 H-pyridin-3-yl)ethyl]-N-propyl-acetamide | 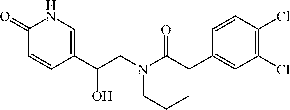 | ¹H NMR (400 MHz, Chloroform-d) δ 7.49 - 7.44 (m, 1H), 7.43 - 7.40 (m, 1H), 7.38 - 7.31 (m, 2H), 7.14 - 7.03 (m, 1H), 6.61 - 6.51 (m, 1H), 4.76 (dd, J = 2.6, 8.0 Hz, 1H), 4.67 - 4.49 (m, 1H), 3.74 - 3.67 (m, 2H), 3.66 - 3.52 (m, 1H), 3.50 - 3.41 (m, 1H), 3.35 -3.01 (m, 2H), 1.62 - 1.49 (m, 2H), 0.98 - 0.83 (m, 3H); LCMS (ESI) m/z⁺: 383.0 [M+H]⁺ | 115 |
| 2-(3,4-dichlorophenyl)-N-[2-hydroxy-2-(3-pyridyl)ethyl]-N-propyl-propanamide | 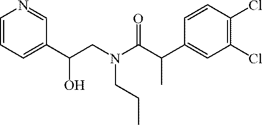 [SMILES: CCCN(CC(O)c1cccnc1)C(=O)Cc1cccnc1] | ¹H NMR (400 MHz, Chloroform-d) δ 8.65 - 8.43 (m, 2H), 7.78 - 7.61 (m, 1H), 7.46 - 7.28 (m, 2H), 7.25 - 7.05 (m, 2H), 5.13 - 4.72 (m, 2H), 3.91 -3.78 (m, 1H), 3.71 - 3.54 (m, 1H), 3.45 (br. d, J = 5.1 Hz, 2H), 2.99 - 2.76 (m, | 117 |
| | | 1H), 1.61 - 1.23 (m, 5H), 0.91 - 0.79 (m, 3H); LCMS (ESI) m/z: 381.0 [M+H]⁺ | |
| 2-(3,4-dichlorophenyl)-N-[2-hydroxy-2-(1-methyl-6-oxo-3-pyridyl)ethyl]-N-propyl-acetamide | 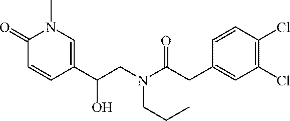 | ¹H NMR (400 MHz, Chloroform-d) δ 7.35 (d, J = 8.2 Hz, 1H), 7.28 (br. d, J = 15.2 Hz, 2H), 7.21 (br. d, J = 2.2 Hz, 1H), 7.19 (s, 1H), 7.03 (br. d, J = 8.2 Hz, 1H), 6.48 (d, J = 9.5 Hz, 1H), 4.67 (br. d, J = 6.2 Hz, 1H), 3.62 (s, 2H), 3.55 (dd, J = 8.2, 14.1 Hz, 1H), 3.45 -3.34 (m, 4H), 3.21 - 3.09 (m, 2H), 1.53 - 1.45 (m, 2H), 0.86 (t, J = 7.4 Hz, 3H); LCMS (ESI) m/z: 397 [M+H]⁺ | 121 |
| 2-(3,4-dichlorophenyl)-N-[2-hydroxy-1-(1-methyl-6-oxo-3-pyridyl)ethyl]-N-propyl-acetamide | 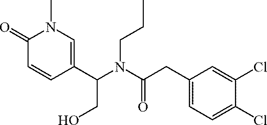 | ¹H NMR (400 MHz, Dimethylsulfoxide-d₆) δ 7.61 (dd, J = 1.8, 14.8 Hz, 1H), 7.58 - 7.54 (m, 1H), 7.51 (s, 1H), 7.37 -7.26 (m, 1H), 7.23 (br. d, J = 8.4 Hz, 1H), 6.34 (d, J = 9.5 Hz, 1H), 5.17 -5.07 (m, 1H), 4.94 - 4.80 (m, 1H), 4.03 - 3.79 (m, 2H), 3.75 (s, 2H), 3.41 (s, 3H), 3.14 (br. t, J = 7.9 Hz, 1H), 3.07 -2.83 (m, 1H), 1.57 - 1.26 (m, 2H), 0.79 (t, J = 7.3 Hz, 2H), 0.65 (t, J = 7.4 Hz, 1H); LCMS (ESI) m/z: 397 [M+H]⁺ | 122 |
| N-[2-hydroxy-2-(3-pyridyl)ethyl]-N-propyl-2-(3-pyridyl)acetamide | 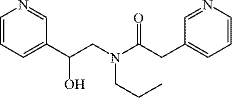 | ¹H NMR (400 MHz, Dimethylsulfoxide-d₆) δ 8.64 (d, J = 1.8 Hz, 1H), 8.56 -8.37 (m, 3H), 7.90 - 7.67 (m, 1H), 7.60 - 7.49 (m, 1H), 7.40 (dd, J = 4.8, 7.8 Hz, 1H), 7.36 - 7.26 (m, 1H), 5.98 -5.53 (m, 1H), 4.95 - 4.79 (m, 1H), 3.83 - 3.46 (m, 3H), 3.45 - 3.35 (m, 2H), 3.28 - 3.12 (m, 1H), 1.59 - 1.42 (m, 2H), 0.87 - 0.75 (m, 3H); LCMS (ESI) m/z: 300.2 [M+H]⁺ | 123 |
| 2-(3,4-dichlorophenyl)-N-[2-(5-fluoro-3-pyridyl)-2-hydroxyethyl]-N-propyl-acetamide | 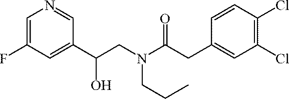 [SMILES: CC(C)(C)OC(=O)N(CCc1ccc(Cl)c(Cl)c1)CC(O)c1cccnc1] | ¹H NMR (400 MHz, Chloroform-d) 8.37 (s, 2H), 7.49 (br. d, J = 9.2 Hz, 1H), 7.42 (d, J = 8.2 Hz, 1H), 7.35 (s, 1H), 7.14 - 7.05 (m, 1H), 5.05 (br. d, J = 7.5 Hz, 1H), 4.93 (br. d, J = 8.8 Hz, 1H), 3.83 - 3.64 (m, 3H), 3.51 (br. d, J = 14.4 Hz, 1H), 3.32 - 3.20 (m, 1H), 3.16 - 3.04 (m, 1H), 1.62 - 1.48 (m, 2H), | 126 |
| | | 1.00 - 0.82 (m, 3H); LCMS (ESI) m/z: 385.1 [M+H]⁺ | |
| N-[2-(4-amino-3-pyridyl)-2-hydroxyethyl]-N-propyl-2-[6-(trifluoromethyl)-3-pyridyl]acetamide | 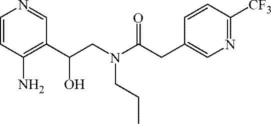 [SMILES: CC(C)(C)OC(=O)NCCCCCN(CC(O)c1cccnc1)C(=O)Cc1ccc(Cl)c(Cl)c1] | ¹H NMR (400 MHz, Methanol-d₄) δ 8.66 - 8.59 (s, 1H), 8.57 - 8.48 (s, 1H), 8.20 - 8.13 (s, 1H), 8.07 - 7.91 (m, 2H), 7.89 - 7.73 (m, 1H), 6.84 (d, J = 6.8 Hz, 1H), 4.96 (d, J = 8.2 Hz, 1H), 4.12 -3.92 (m, 2H), 3.91 - 3.84 (m, 1H), 3.76 - 3.46 (m, 2H), 3.12 - 3.00 (m, 1H), 1.83 - 1.55 (m, 2H), 1.04 - 0.85 (t, 3H); LCMS (ESI) m/z: 383.1 [M+H]⁺ | 136 |
| Tert-butyl N-[2-hydroxy-2-(3-pyridyl)ethyl]-N-[2-(2-naphthyl)ethyl]carba mate | 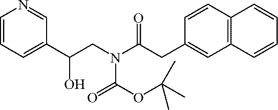 | ¹H NMR (400 MHz, CHLOROFORM-d) δ 8.59 - 8.48 (m, 2H), 7.84 - 7.75 (m, 3H), 7.74 - 7.53 (m, 2H), 7.50 - 7.40 (m, 2H), 7.27 (s, 2H), 4.93 (br d, J=3.5 Hz, 1H), 4.75 (br s, 1H), 3.67 - 3.21 (m, 4H), 3.09 - 2.82 (m, 2H), 1.44 (s, 9H). LCMS (ESI) m/z: 393.3 [M+H]+ | 137 |
| Tert-butyl N-[2-(3,4-dichlorophenyl)ethyl] -N-[2-hydroxy-2-(3-pyridyl)ethyl]carbam ate | 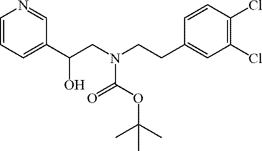 | ¹H NMR (400 MHz, DMSO-d₆) δ = 8.55 - 8.49 (m, 1H), 8.48 - 8.42 (m, 1H), 7.74 - 7.66 (m, 1H), 7.53 - 7.46 (d, J = 8 Hz, 1H), 7.44 -7.38 (m, 1H), 7.37 -7.29 (m, 1H), 7.19 - 7.12 (m, 1H), 5.42 - 5.33 (d, J = 4.4 Hz, 1H), 4.87 - 4.76 (m, 1H), 3.49 - 3.24 (m, 4H), 2.84 -2.72 (m, 2H), 1.29 (s, 9H); LCMS (ESI) m/z: 411.1 [M+H]+ | 138 |
| tert-butyl N-[5-[[2-(3,4-dichlorophenyl)acety l]-[2-hydroxy-2-(3-pyridyl)ethyl]amino]p entyl]carbamate | 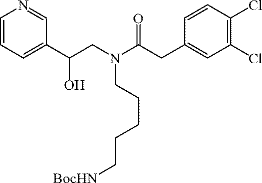 | ¹H NMR (400 MHz, METHANOL-d₄) δ 8.64 - 8.53 (m, 1H), 8.51 - 8.41 (m, 1H), 7.97 - 7.82 (m, 1H), 7.50 - 7.34 (m, 3H), 7.19 - 7.09 (m, 1H), 5.00 (ddd, J=4.1, 8.3, 12.0 Hz, 1H), 3.90 - 3.64 (m, 3H), 3.63 - 3.36 (m, 3H), 3.11 -2.94 (m, 2H), 1.66 - 1.52 (m, 2H), 1.51 - 1.45 (m, 2H), 1.42 (s, 9H), 1.35 - 1.23 (m, 2H); LCMS (ESI) m/z: 510.3 [M+H]+ | 139 |

### Example 3: Preparation of 2-(3,4-Dichlorophenyl)-N-[(2R)-2-Hydroxy-2-(3-Pyridyl)ethyl]-N-Propyl-Acetamide (Compound 6)

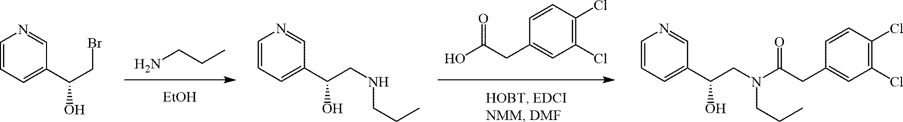 [SMILES: CCCN(C[C@@H](O)c1cccnc1)C(=O)Cc1ccc(Cl)c(Cl)c1]

### Step 1: Preparation of (1R)-2-(Propylamino)-1-(3-Pyridyl)Ethanol

To a solution of (1R)-2-bromo-1-(3-pyridyl)ethanol (500 mg, 2.47 mmol) in ethanol (5 mL) was added propan-1-amine (293 mg, 4.95 mmol). The mixture was stirred at 80° C. for 4 h. Then an additional amount of propan-1-amine (146 mg, 2.47 mmol) was added to the reaction mixture, the resultant solution was stirred at 80° C. for 12 h. The reaction mixture was then concentrated under reduced pressure and purified by prep-HPLC (Waters Xbridge Prep OBD C18 150 × 40 10 um column; 1-25% acetonitrile in a 10 mM ammonium bicarbonate solution in water, 11 min gradient) to give (1R)-2-(propylamino)-1-(3-pyridyl)ethanol (100 mg, ee% = 87%) as a yellow thick oil.

¹H NMR (400 MHz, Chloroform-d) δ 8.60 (d, J = 2.0 Hz, 1H), 8.53 (dd, J = 1.5, 4.9 Hz, 1H), 7.76 - 7.72 (m, 1H), 7.31 = 7.28 (m, 1H), 4.72 (dd, J = 3.5, 9.3 Hz, 1H), 2.94 (dd, J = 3.6, 12.2 Hz, 1H), 2.72 - 2.57 (m, 3H), 1.53 (sxt, J = 7.3 Hz, 2H), 0.94 (t, J = 7.4 Hz, 3H); LCMS (ESI) m/z: 181.1 [M+H]⁺.

### Step 2: Preparation of 2-(3,4-Dichlorophenyl)-N-[(2R)-2-Hydroxy-2-(3-Pyridyl)ethyl]-N-Propyl-Acetamide

To a solution of 2-(3,4-dichlorophenyl)acetic acid (114 mg, 555 µmol) in dimethylformamide (1 mL) were added N-methylmorpholine (140 mg, 1.39 mmol), N-(3-dimethylaminopropyl)-N′-ethylcarbodiimide (106 mg, 555 µmol), 1-hydroxybenzotriazole (75 mg, 555 µmol) and (1R)-2-(propylamino)-1-(3-pyridyl)ethanol (50 mg, 277 µmol) at 25° C. The mixture was stirred at 25° C. for 12 h, solution was filtered and purified by prep-HPLC (Kromasil 150 × 25 mm × 10 um column; 30-50% acetonitrile in an a 0.04% ammonia and 10 mM ammonium bicarbonate solution in water, 10 min gradient) to give 2-(3,4-dichlorophenyl)-N-[(2R)-2-hydroxy-2-(3-pyridyl)ethyl]-N-propyl-acetamide (10 mg, 28 µmol, 10%, ee% = 90% ) as a pale yellow thick oil.

¹H NMR (400 MHz, Methanol-d₄) δ 8.67 - 8.41 (m, 2H), 7.97 - 7.80 (m, 1H), 7.50 - 7.34 (m, 3H), 7.17 -7.09 (m, 1H), 5.00 (ddd, J = 4.1, 8.3, 12.3 Hz, 1H), 3.90 - 3.65 (m, 3H), 3.59 - 3.34 (m, 3H), 1.67 - 1.53 (m, 2H), 0.90 (dt, J = 5.0, 7.3 Hz, 3H); LCMS (ESI) m/z: 367.0 [M+H]⁺.

### Example 4: Preparation of N-[(2S)-2-Hydroxy-2-(3-Pyridyl)Ethyl]-2-(2-Naphthyl)-N-Propyl-Acetamide (Compound 7)

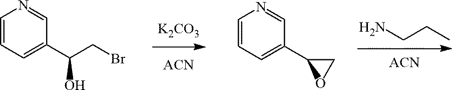 [SMILES: CCCN(C[C@@H](O)c1cccnc1)C(=O)Cc1ccc(OC)c(Cl)c1]

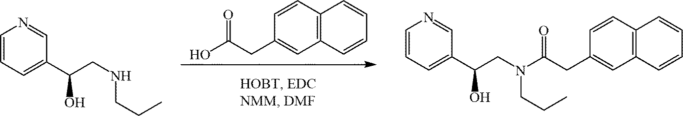 [SMILES: CC(C)N(C[C@@H](O)c1cccnc1)C(=O)Cc1ccc(C(F)(F)F)nc1]

### Step 1: Preparation of (1S)-2-(Propylamino)-1-(3-Pyridyl)Ethanol

To a solution of (1S)-2-bromo-1-(3-pyridyl)ethanol (1.00 g, 4.95 mmol) in acetonitrile (10 mL) was added potassium carbonate (2.74 g, 19.80 mmol). The mixture was stirred at 90° C. for 2 h and the reaction solution was filtered to give a solution of 3-[(2S)-oxiran-2-yl]pyridine in acetonitrile. Propan-1-amine (878 mg, 14.85 mmol) was added to above solution and the mixture was stirred at 90° C. for 24 h. The reaction mixture was filtered and the filtrate was purified by prep-HPLC (Waters Xbridge Prep OBD C18 50 × 40 10 um column; 1-20% acetonitrile in a 0.04% ammonia and 10 mM ammonium bicarbonate solution in water, 11 min gradient) to give (1S)-2-(propylamino)-1-(3-pyridyl)ethanol (430 mg, crude) as a pale yellow thick oil.

### Step 2: Preparation of N-[(2S)-2-Hydroxy-2-(3-Pyridyl)Ethyl]-2-(2-Naphthyl)-N-Propyl-Acetamide

Compound 7 was synthesized according to the synthetic procedure reported for the Preparation of Compound 1. The compound N-[(2S)-2-hydroxy-2-(3-pyridyl)ethyl]-2-(2-naphthyl)-N-propyl-acetamide (10 mg, 29 µmol, ee% = 91%, HCl,) was obtained as a pale yellow thick oil. ¹H NMR (400 MHz, Methanol-d₄) δ 8.98 - 8.82 (m, 1H), 8.78 - 8.53 (m, 2H), 8.10 - 7.75 (m, 4H), 7.68 (s, 1H), 7.53 - 7.41 (m, 2H), 7.40 - 7.26 (m, 1H), 5.27 - 5.12 (m, 1H), 4.19 - 3.87 (m, 2H), 3.85 - 3.73 (m, 1H), 3.69 - 3.49 (m, 3H), 1.73 - 1.53 (m, 2H), 0.96 - 0.87 (m, 3H); LCMS (ESI) m/z: 349.1 [M+H]⁺.

The following compounds were synthesized according to the protocol described above.

| Name | Structure | NMR, MS | Comp d# |
|---|---|---|---|
| 2-(3,4-dichlorophenyl)-N-[(2S)-2-hydroxy-2-(3-pyridyl)ethyl]-N-propyl-acetamide | 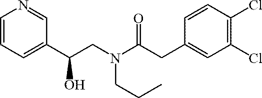 [SMILES: O=C(Cc1ccc(C(F)(F)F)nc1)N(Cc1ccccc1)C[C@@H](O)c1cccnc1] | ¹H NMR (400 MHz, Methanol-d₄) δ 9.03 -8.86 (m, 1H), 8.85 - 8.61 (m, 2H), 8.19 -7.94 (m, 1H), 7.51 - 7.32 (m, 2H), 7.22 -7.07 (m, 1H), 5.20 (br. dd, J = 4.0, 7.0 Hz, 1H), 4.02 - 3.72 (m, 3H), 3.67 - 3.46 (m, 3H), 1.73 - 1.57 (m, 2H), 0.99 - 0.86 (m, 3H); LCMS (ESI) m/z: 367.0 [M+H]⁺. | 8 |
| 2-(3-chloro-4-methoxy-phenyl)-N-[(2S)-2-hydroxy-2-(3-pyridyl)ethyl]-N-propyl-acetamide | 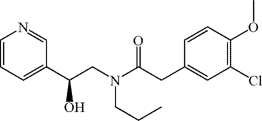 [SMILES: O=C(Cc1ccc(F)c(Cl)c1)N(CC1COC1)C[C@@H](O)c1cccnc1] | ¹H NMR (400 MHz, Chloroform-d) δ 8.63 -8.55 (m, 1H), 8.53 (dd, J = 1.4, 4.7 Hz, 1H), 7.74 (br. d, J = 7.8 Hz, 1H), 7.30 -7.24 (m, 3H), 7.17 - 7.05 (m, 1H), 6.95 -6.84 (m, 1H), 5.06 - 5.00 (m, 1H), 4.99 -4.89 (m, 1H), 3.91 (s, 3H), 3.76 (dd, J = 8.0, 14.4 Hz, 1H), 3.66 (s, 2H), 3.50 (dd, J = 2.2, 14.4 Hz, 1H), 3.29 - 3.18 (m, 1H), 3.12 = 3.01 (m, 1H), 1.60 - 1.45 (m, 2H), 0.90 (t, J = 7.2 Hz, 3H); LCMS (ESI) m/z: 363.1 [M+H]⁺. | 12 |
| 2-(3,4-dichlorophenyl)-N-[(2S)-2-hydroxy-2-(3-pyridyl)ethyl]-N-(oxetan-3-yl)acetamide | 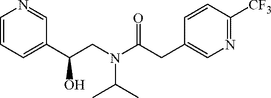 [SMILES: O=C(Cc1ccc(OC(F)(F)F)cn1)N(CC1COC1)C[C@@H](O)c1cccnc1] | ¹H NMR (400 MHz, Chloroform-d) δ 8.63 (br. d, J = 7.9 Hz, 2H), 8.57 (dd, J = 1.3, 4.8 Hz, 1H), 7.87 (br. d, J = 7.6 Hz, 1H), 7.81 (br. d, J = 7.8 Hz, 1H), 7.73 (d, J = 8.1 Hz, 1H), 7.33 (dd, J = 4.9, 7.8 Hz, 1H), 5.35 (br. s, 1H), 4.94 (br. d, J = 8.3 Hz, 1H), 4.29 - 4.10 (m, 1H), 4.02 - 3.77 (m, 3H), 3.24 (dd, J = 2.0, 14.7 Hz, 1H), 1.35 (d, J = 6.7 Hz, 3H), 1.16 (d, J = 6.6 Hz, 3H); LCMS (ESI) m/z: 368.0 [M+H]⁺. | 56 |
| (S)-N-benzyl-N-(2-hydroxy-2-(pyridin-3-yl)ethyl)-2-(6-(trifluoromethyl)pyri din-3-yl)acetamide | 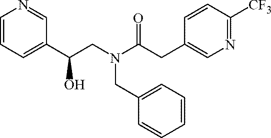 | ¹H NMR (400 MHz, Dimethylsulfoxide-d₆) δ 8.63 - 8.51 (m, 1H), 8.50 - 8.49 (m, 2H),7.86 - 7.80 (m, 3H), 7.40 - 7.22 (m, 6H), 6.00 (d, J = 3.9 Hz, 1H), 5.02 -5.00 (m, 1H), 4.92 (td, J = 4.3, 8.1 Hz, 1H), 4.82 - 4.70 (m, 1H), 4.53 (d, J = 15.2 Hz, 1H), 4.14 (d, J = 16.1 Hz, 1H), 3.84 - 3.67 (m, 1H), 3.50 -3.38 (m, 1H); LCMS (ESI) m/z: 416.1 [M+H]⁺. | 64 |
| 2-(3-chloro-4-fluoro-phenyl)-N-[(2S)-2-hydroxy-2-(3-pyridyl)ethyl]-N-(oxetan-3-ylmethyl)acetamide | 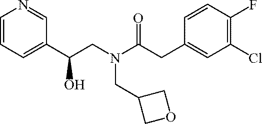 [SMILES: O=C(Cc1ccc(Cl)c(Cl)c1)N(C[C@@H](O)c1cccnc1)C1CCC1] | ¹H NMR (400 MHz, Chloroform-d) δ 8.65 -8.45 (m, 2H), 7.72 (br. d, J = 7.9 Hz, 1H), 7.39 - 7.27 (m, 1.5H), 7.20 (br. d, J = 6.4 Hz, 0.5H), 7.16 - 7.02 (m, 2H), 5.04 - 4.87 (m, 1H), 4.83 - 4.67 (m, 2H), 4.49 (td, J = 6.2, 12.2 Hz, 0.7H), 4.34 (q, J = 6.4 Hz, 1.3H), 4.22 (br. s, 0.5H), 3.95 (dd, J = 7.2, 14.0 Hz, 0.5H), 3.79 - 3.72 (m, 2H), 3.72 -3.62 (m, 2H), 3.60 - 3.51 (m, 1.7H), 3.37 -3.16 (m, 1.3H); LCMS (ESI) m/z: 379.1 [M+H]⁺. | 65 |
| N-[(2S)-2-hydroxy-2-(3-pyridyl)ethyl]-N-(oxetan-3-ylmethyl)-2-[5-(trifluoromethoxy)-2-pyridyl]acetamide | 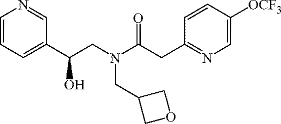 | ¹H NMR (400 MHz, Chloroform-d) δ 8.68 -8.53 (m, 2H), 8.51 - 8.42 (m, 1H), 7.81 -7.69 (m, 1H), 7.64 - 7.55 (m, 1H), 7.46 -7.39 (m, 1H), 7.37 - 7.28 (m, 1H), 5.17 -5.01 (m, 1H), 4.97 - 4.71 (m, 2.4H), 4.60 -4.35 (m, 2.6H), 4.22 - 3.94 (m, 2H), 3.93 -3.72 (m, 2H), 3.66 - 3.56 (m, 1H), 3.54 -3.43 (m, 1H), 3.41 - 3.22 (m, 1H); LCMS (ESI) m/z: 412.2 [M+H]⁺. | 66 |
| N-(6,7-dichlorotetralin-2-yl)-N-[(2S)-2-hydroxy-2-(3-pyridyl)ethyl]propan amide | 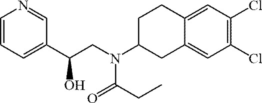 [SMILES: O=C(Cc1ccc(C(F)(F)F)nc1)N(CC1COC1)C[C@@H](O)c1cccnc1] | ¹H NMR (400 MHz, Chloroform-d) δ 8.67 -8.47 (m, 2H), 7.81 (br. t, J = 7.2 Hz, 1H), 7.36 - 7.28 (m, 1H), 7.24 - 7.06 (m, 2H), 5.76 - 5.52 (m, 1H), 5.05 - 4.85 (m, 1H), 4.16 - 3.95 (m, 1H), 3.92 - 3.73 (m, 1H), 3.40 - 3.12 (m, 1H), 3.06 - 2.39 (m, 6H), 2.32 - 2.12 (m, 1H), 1.98 - 1.69 (m, 1H), 1.23 (dt, J = 3.2, 7.3 Hz, 3H); LCMS (ESI) m/z: 393.1 [M+H]⁺. | 70 |
| N-cyclobutyl-2-(3,4-dichlorophenyl)-N-[(2S)-2-hydroxy-2-(3-pyridyl)ethyl]aceta mide | 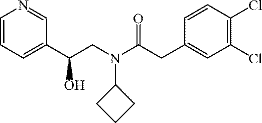 [SMILES: O=C(Cc1ccc(C(F)(F)F)nc1)N(C[C@@H](O)c1cccnc1)C1CCC1] | ¹H NMR (400 MHz, Chloroform-d) δ 8.61 (s, 1H), 8.55 (br. d, J = 3.5 Hz, 1H), 7.78 (br. d, J = 7.9 Hz, 1H), 7.43 (d, J = 8.4 Hz, 1H), 7.35 (s, 1H), 7.31 (dd, J = 4.9, 7.5 Hz, 1H), 7.10 (br. d, J = 7.9 Hz, 1H), 4.98 (br. s, 1H), 4.92 (br. d, J = 7.9 Hz, 1H), 4.34 -4.19 (m, 1H), 3.86 (dd, J = 8.7, 14.7 Hz, 1H), 3.79 - 3.68 (m, 2H), 3.50 (dd, J = 1.7, 14.7 Hz, 1H), 2.29 - 2.08 (m, 2H), 2.07 -1.90 (m, 2H), 1.77 - 1.65 (m, 2H); LCMS (ESI) m/z: 379.0 [M+H]⁺ | 71 |
| (S)-N-(2-hydroxy-2-(pyridin-3-yl)ethyl)-N-isopropyl-2-(6-(trifluoromethyl)pyri din-3-yl)acetamide | 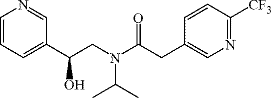 [SMILES: COC(=O)N(CCc1ccc(C(F)(F)F)nc1)C[C@@H](O)c1cccnc1] | ¹H NMR (400 MHz, Chloroform-d) δ 8.62-8.60 (d, J = 8, 1 H), 8.55-8.54 (d, J = 3.6, 1 H), 7.86-7.84(m, 1 H), 7.80-7.78(m, 1H), 7.71-7.69 (d,J = 8, 1H), 7.32-7.29 (m, 1H), 5.40 (br. s, 1H), 4.93-4.91 (d, 1H), 4.21-4.18 (m, 1H), 3.91-3.78 (m, 3H), 3.23-3.20 (m, 1H), 1.27-1.25 (d, 3H), 1.15-1.13 (d, 3H); LCMS (ESI) m/z: 368.0 [M+H]⁺ | 72 |
| N-[(2S)-2-hydroxy-2-(3-pyridyl)ethyl]-N-(oxetan-3-ylmethyl)-2-[6-(trifluoromethyl)-3-pyridyl]acetamide | 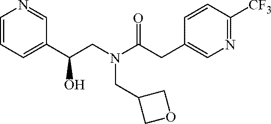 [SMILES: CC1(CN(C[C@@H](O)c2cccnc2)C(=O)Cc2ccc(C(F)(F)F)nc2)CC1] | ¹H NMR (400 MHz, Methanol-d₄) δ 8.66 -8.55(m, 1H), 8.51-8.42 (m, 2H), 7.99-7.97 (m, 1H), 7.92-7.85 (m, 1H), 7.77-7.75 (m, 1H), 7.49-7.35(m, 1 H), 5.04-4.99(m, 1 H), 4.74-4.71(m, 2H), 4.54-4.51(m, 2H), 4.13-3.75 (m, 4H), 3.70-3.65 (m, 1H), 3.53 -3.31 (m, 2H); LCMS (ESI) m/z: 396.0 [M+H]⁺ | 73 |
| N-cyclobutyl-N-[(2S)-2-hydroxy-2-(3-pyridyl)ethyl]-2-[6-(trifluoromethyl)-3-pyridyl]acetamide | 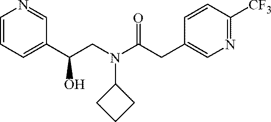 [SMILES: N#Cc1ccc(CC(=O)N(C[C@@H](O)c2cccnc2)C2CCC2)cc1Cl] | ¹H NMR (400 MHz, Chloroform-d) δ 8.69 -8.48 (m, 3H), 7.90 - 7.74 (m, 2H), 7.73 -7.60 (m, 1H), 7.34 - 7.28 (m, 1H), 5.03 -4.89 (m, 1H), 4.41 - 4.23 (m, 1H), 3.96 -3.77 (m, 3H), 3.60 - 3.43 (m, 1H), 2.40 - | 74 |
| | | 1.99 (m, 5H), 1.83 - 1.72 (m, 2H; LCMS (ESI) m/z: 380.0 [M+H]⁺ | |
| Methyl N-[(2S)-2-hydroxy-2-(3-pyridyl)ethyl]-N-[2-[6-(trifluoromethyl)-3-pyridyl]ethyl]carba mate | 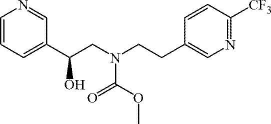 [SMILES: O=C(Cc1ccc(C(F)(F)F)nc1)N(C[C@@H](O)c1cccnc1)C1COC1] | ¹H NMR (400 MHz, Methanol-d4) δ 8.57-8.53 (m, 2H), 8.45-8.44 (d, J = 4.4 Hz, 1H), 7.90 - 7.87 (m, 2H), 7.75-7.73 (d, J = 8, 1H), 7.44-7.41 (m, 1H), 4.98-4.93(m, 1H), 3.66 - 3.51 (m, 6H), 3.49-3.41 (m, 1H), 3.02-2.98 (m, 2H); LCMS (ESI) m/z: 370.1 [M+H]⁺ | 75 |
| N-[(2S)-2-hydroxy-2-(3-pyridyl)ethyl]-N-[(1-methylcyclopropyl) methyl]-2-[6-(trifluoromethyl)-3-pyridyl]acetamide | 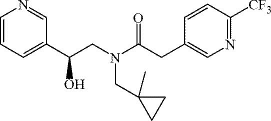 [SMILES: O=C(Cc1ccc(C(F)(F)F)nc1)N(CC1CCOCC1)C[C@@H](O)c1cccnc1] | ¹H NMR (400 MHz, Chloroform-d) δ 8.67 -8.56 (m, 2H), 8.54 (d, J = 3.5 Hz, 1H), 7.88 - 7.62 (m, 3H), 7.40 - 7.28 (m, 1H), 5.09 (br. d, J = 8.7 Hz, 1H), 4.41 (br. s, 1H), 4.16 - 3.94 (m, 1H), 3.92 - 3.78 (m, 2H), 3.55 (dd, J = 4.0, 15.0 Hz, 2H), 3.18 - 3.00 (m, 1H), 1.11 (s, 3H), 0.61 - 0.35 (m, 4H); LCMS (ESI) m/z: 394.2 [M+H]⁺ | 76 |
| 2-(3-chloro-4-cyano-phenyl)-N-cyclobutyl-N-[(2S)-2-hydroxy-2-(3-pyridyl)ethyl]aceta mide | 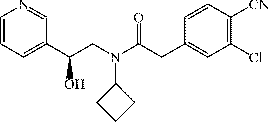 [SMILES: N#Cc1ccc(CC(=O)N(CC2COC2)C[C@@H](O)c2cccnc2)cc1Cl] | ¹H NMR (400 MHz, Dimethylsulfoxide-d₆) δ 8.67 - 8.38 (m, 2H), 7.93 - 7.66 (m, 2H), 7.59 - 7.46 (m, 1H), 7.44 - 7.25 (m, 2H), 6.00 - 5.49 (m, 1H), 4.87 - 4.63 (m, 1H), 4.40 - 4.21 (m, 1H), 4.05 - 3.37 (m, 4H), 2.30 - 1.76 (m, 4H), 1.69 - 1.41 (m, 2H); LCMS (ESI) m/z: 370.1 [M+H]⁺ | 77 |
| N-[(2S)-2-hydroxy-2-(3-pyridyl)ethyl]-N-(oxetan-3-yl)-2-[6-(trifluoromethyl)-3-pyridyl]acetamide | 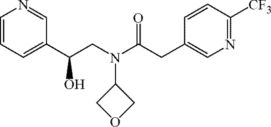 [SMILES: O=C(Cc1ccc(C(F)(F)F)nc1)N(CC1CCNCC1)C[C@@H](O)c1cccnc1] | ¹H NMR (400 MHz, Chloroform-d) δ 8.69 -8.47 (m, 3H), 7.85 - 7.60 (m, 3H), 7.41 -7.29 (m, 1H), 5.25 - 5.05 (m, 1H), 5.01 -4.59 (m, 5H), 4.16 - 3.68 (m, 4H), 3.60 -3.44 (m, 1H); LCMS (ESI) m/z: 382.1 [M+H]⁺ | 78 |
| N-[(2S)-2-hydroxy-2-(3-pyridyl)ethyl]-N-(tetrahydropyran-4-ylmethyl)-2-[6-(trifluoromethyl)-3-pyridyl]acetamide | 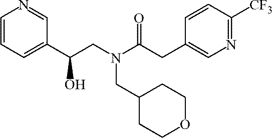 [SMILES: O=C(Cc1ccc(Cl)c(Cl)c1)N(C[C@@H](O)c1cccnc1)C1CC1] | ¹H NMR (400 MHz, Chloroform-d) δ 8.70 -8.49 (m, 3H), 7.86 - 7.61 (m, 3H), 7.40 -7.27 (m, 1H), 5.11 - 4.97 (m, 1H), 4.24 -3.89 (m, 3H), 3.88 - 3.49 (m, 4H), 3.42 -3.00 (m, 4H), 2.06 - 1.81 (m, 1H), 1.62 -1.47 (m, 2H), 1.42 - 1.26 (m, 2H); LCMS (ESI) m/z: 424.3 [M+H]⁺ | 79 |
| 2-(3-chloro-4-cyano-phenyl)-N-[(2S)-2-hydroxy-2-(3-pyridyl)ethyl]-N- | 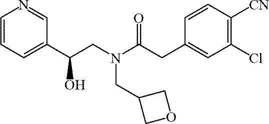 [SMILES: CCC(=O)N(C[C@@H](O)c1cccnc1)C1Cc2cnc(C(F)(F)F)cc2C1] | ¹H NMR (400 MHz, Chloroform-d) δ 8.61 -8.47 (m, 2H), 7.78 - 7.70 (m, 1H), 7.67 -7.56 (m, 1H), 7.45 - 7.28 (m, 2H), 7.26 -7.15 (m, 1H), 5.07 - 4.89 (m, 1H), 4.86 -4.68 (m, 2H), 4.47 (td, J = 6.4, 12.6 Hz, | 80 |
| (oxetan-3-ylmethyl)acetamide | | 1H), 4.36 (q, J = 6.6 Hz, 1H), 4.00 - 3.91 (m, 1H), 3.87 - 3.42 (m, 5.5H), 3.37 - 3.20 (m, 1.5H); LCMS (ESI) m/z: 386.1 [M+H]⁺ | |
| N-[(2S)-2-hydroxy-2-(3-pyridyl)ethyl]-N-(4-piperidylmethyl)-2-[6-(trifluoromethyl)-3-pyridyl]acetamide | 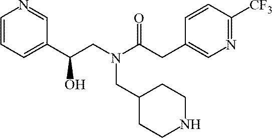 | ¹H NMR (400 MHz, CHLOROFORM-d) δ = 8.64 - 8.45 (m, 3H), 7.83 - 7.69 (m, 2H), 7.69 - 7.56 (m, 1H), 7.27 (s, 1H), 5.10 -4.89 (m, 1H), 4.21 - 4.02 (m, 1H), 3.84 -3.68 (m, 2H), 3.67 - 3.51 (m, 2H), 3.38 -3.20 (m, 2H), 3.15 - 2.96 (m, 2H), 2.64 -2.43 (m, 2H), 1.79 - 1.51 (m, 3H), 1.23 -1.07 (m, 2H). LCMS (ESI) for C21 H25F3N402 [M+H]+: 423.2 | 81 |
| N-cyclopropyl-2-(3,4-dichlorophenyl)-N-[(2S)-2-hydroxy-2-(3-pyridyl)ethyl]aceta mide | 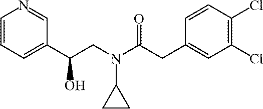 | ¹H NMR (400 MHz, Chloroform-d) δ 8.57 (s, 1H), 8.52 (br. d, J = 4.5 Hz, 1H), 7.71 (br. d, J = 7.8 Hz, 1H), 7.44 - 7.34 (m, 2H), 7.28 - 7.25 (br. s, 1H), 7.12 (br. d, J = 8.2 Hz, 1H), 4.98 (br. d, J = 6.1 Hz, 1H), 4.44 (br. s, 1H), 3.96 - 3.81 (m, 2H), 3.80 - 3.71 (m, 1H), 3.69 - 3.61 (m, 1H), 2.57 (br. d, J = 3.4 Hz, 1H), 0.99 - 0.85 (m, 2H), 0.77 (br. d, J = 2.4 Hz, 2H); LCMS (ESI) m/z: 365.1 [M+H]⁺ | 86 |
| N-[(2S)-2-hydroxy-2-(3-pyridyl)ethyl]-N-[3-(trifluoromethyl)-6,7-dihydro-5H-cyclopenta[c]pyridin -6-yl]propanamide | 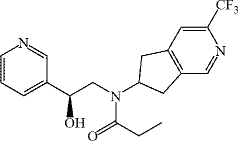 | ¹H NMR (400 MHz, CHLOROFORM-d) δ = 8.78 - 8.36 (m, 3H), 7.73 - 7.48 (m, 2H), 7.40 - 7.27 (m, 1H), 5.03 - 4.84 (m, 2H), 3.78 - 2.94 (m, 6H), 2.77 - 2.23 (m, 2H), 1.33 - 1.05 (m, 3H). LCMS (ESI) for C19H20F3N3O2 [M+H]+: 380.2. | 93 |
| N-[(2S)-2-hydroxy-2-(3-pyridyl)ethyl]-N-isopropyl-2-[2-(trifluoromethyl)pyri midin-5-yl]acetamide | 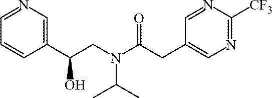 | ¹H NMR (400 MHz, Chloroform-d) δ 8.87 -8.76 (m, 2H), 8.58 - 8.52 (m, 2H), 7.83 -7.70 (m, 1H), 7.37 (br. dd, J = 4.8, 7.8 Hz, 1H), 5.05 - 4.90 (m, 1H), 4.27 - 4.14 (m, 1H), 3.97 - 3.58 (m, 3H), 3.35 (br. dd, J = 2.8, 15.8 Hz, 1H), 1.42 - 1.30 (m, 3H), 1.29 - 1.19 (m, 4H); LCMS (ESI) m/z: 369.1 [M+H]⁺ | 94 |
| 6,7-dichloro-N-[(2S)-2-hydroxy-2-(3-pyridyl)ethyl]-N-propyl-tetralin-2-carboxamide | 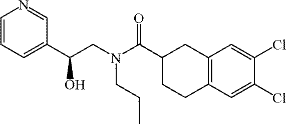 [SMILES: CCCN(C[C@@H](O)c1cccnc1)C(=O)c1nc2cc(Cl)c(Cl)cc2[nH]1] | ¹H NMR (400 MHz, Methanol-d₄) δ 8.61 -8.51 (m, 1H), 8.49 - 8.43 (m, 1H), 7.95 -7.85 (m, 1H), 7.49 - 7.39 (m, 1H), 7.30 -7.16 (m, 2H), 5.09 - 4.91 (m, 2H), 3.85 -3.63 (m, 2H), 3.60 - 3.34 (m, 3H), 3.10 - | 98 |
| | | 2.64 (m, 4H), 2.02 - 1.68 (m, 2H), 1.67 -1.53 (m, 2H), 0.98 - 0.85 (m, 3H); LCMS (ESI) m/z: 407.1 [M+H]⁺ | |
| 2-(3,4-dimethylphenyl)-N-[(2S)-2-hydroxy-2-(3-pyridyl)ethyl]-N-propyl-acetamide | 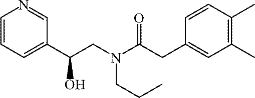 | ¹H NMR (400 MHz, Chloroform-d) δ 8.61 -8.54 (m, 1H), 8.54 - 8.47 (m, 1H), 7.78 -7.65 (m, 1H), 7.26 - 7.20 (m, 1H), 7.16 -6.92 (m, 3H), 5.33 - 5.13 (m, 1H), 5.01 (br. d, J = 6.4 Hz, 1H), 3.83 - 3.72 (m, 1H), 3.68 (s, 2H), 3.47 (dd, J = 2.2, 14.4 Hz, 1H), 3.33 - 3.17 (m, 1H), 3.03 (ddd, J = 6.4, 8.9, 14.8 Hz, 1H), 2.32 - 2.20 (m, 6H), 1.60 - 1.44 (m, 2H), 0.87 (t, J = 7.2 Hz, 3H); LCMS (ESI) m/z: 327.2 [M+H]^{+.} | 99 |
| 2-(3-chloro-4-cyano-phenyl)-N-[(2S)-2-hydroxy-2-(3-pyridyl)ethyl]-N-propyl-acetamide | 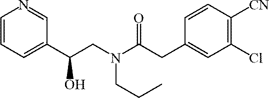 | ¹H NMR (400 MHz, Chloroform-d) δ 8.66 -8.57 (m, 1H), 8.56 - 8.51 (m, 1H), 7.75 (td, J = 1.6, 7.8 Hz, 1H), 7.69 - 7.58 (m, 1H), 7.47 - 7.42 (m, 1H), 7.40 - 7.27 (m, 1H), 7.26 - 7.20 (m, 1H), 5.08 - 4.94 (m, 1H), 4.55 - 4.34 (m, 1H), 3.80 - 3.61 (m, 3H), 3.53 (dd, J = 2.6, 14.2 Hz, 1H), 3.38 - 3.12 (m, 2H), 1.62 - 1.53 (m, 2H), 0.99 - 0.87 (m, 3H); LCMS (ESI) m/z: 358.1 [M+H]^{+.} | 100 |
| 5,6-dichloro-N-[(2S)-2-hydroxy-2-(3-pyridyl)ethyl]-N-propyl-1 H-benzimidazole-2-carboxamide | 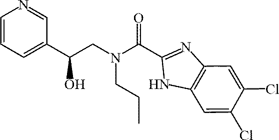 | ¹H NMR (400 MHz, Methanol-d₄) δ 8.66 -8.59 (m, 1H), 8.50 - 8.37 (m, 1H), 8.01 -7.79 (m, 3H), 7.49 - 7.32 (m, 1H), 5.22 -5.12 (m, 1H), 4.46 - 4.37 (m, 1H), 4.26 -4.15 (m, 1H), 4.05 - 3.83 (m, 1H), 3.78 -3.58 (m, 2H), 1.83 - 1.63 (m, 2H), 1.05 -0.82 (m, 3H); LCMS (ESI) m/z: 393.0 [M+H]⁺ | 101 |
| N-[(2S)-2-hydroxy-2-(3-pyridyl)ethyl]-N-propyl-2-pyrimidin-5-yl-acetamide | 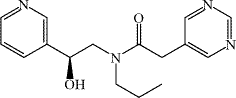 | ¹H NMR (400 MHz, Chloroform-d) δ 9.21 -9.04 (m, 1H), 8.71 - 8.50 (m, 4H), 7.75 (br. d, J = 7.8 Hz, 1H), 7.41 - 7.27 (m, 1H), 5.10 - 4.97 (m, 1H), 4.05 - 3.51 (m, 4H), 3.44 - 3.10 (m, 2H), 1.74 - 1.60 (m, 2H), 1.03 - 0.87 (m, 3H); LCMS (ESI) m/z: 301.2 [M+H]⁺ | 104 |
| 2-[3-fluoro-4-(trifluoromethyl)phe nyl]-N-[(2S)-2-hydroxy-2-(3- | 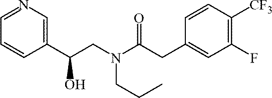 [SMILES: CCCN(C[C@@H](O)c1cccnc1)C(=O)Cc1ccc(F)cn1] | ¹H NMR (400 MHz, Chloroform-d) δ 8.64 -8.47 (m, 2H), 7.72 (br. d, J = 7.7 Hz, 1H), 7.57 (br. t, J = 7.5 Hz, 1H), 7.37 - 7.22 (m, 1H), 7.17 - 7.03 (m, 2H), 5.07 - 4.91 (m, 1H), 3.98 - 3.83 (m, 1H), 3.81 - 3.67 (m, | 105 |
| pyridyl)ethyl]-N-propyl-acetamide | | 3H), 3.57 - 3.48 (m, 1H), 3.34 - 3.08 (m, 2H), 1.64 - 1.54 (m, 2H), 0.96 - 0.84 (m, 3H); LCMS (ESI) [M+H]⁺: 385.0 | |
| N-[(2S)-2-hydroxy-2-(3-pyridyl)ethyl]-N-propyl-2-[5-(trifluoromethyl)-2-pyridyl]acetamide | 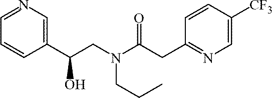 | ¹H NMR (400 MHz, Dimethylsulfoxide-d₆) δ 8.85 (br. d, J = 10.4 Hz, 1H), 8.68 - 8.40 (m, 2H), 8.12 (br. t, J = 6.1 Hz, 1H), 7.88 -7.66 (m, 1H), 7.55 - 7.43 (m, 1H), 7.42 -7.27 (m, 1H), 5.90 - 5.54 (m, 1H), 4.96 -4.81 (m, 1H), 4.03 (s, 1H), 3.98 (s, 1H), 3.74 - 3.46 (m, 1H), 3.46 - 3.33 (m, 2H), 3.27 - 3.12 (m, 1H), 1.61 - 1.41 (m, 2H), 0.87 - 0.75 (m, 3H); LCMS (ESI) m/z: 368.0 [M+H]⁺ | 106 |
| 5,6-dichloro-N-[(2S)-2-hydroxy-2-(3-pyridyl)ethyl]-N-propyl-isoindoline-2-carboxamide | 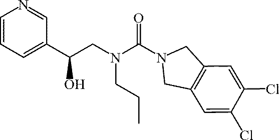 | ¹H NMR (400 MHz, Chloroform-d) δ 8.61 (br. s, 1H), 8.54 (br. d, J = 3.3 Hz, 1H), 7.82 (br. d, J = 7.9 Hz, 1H), 7.37 (s, 2H), 7.30 (dd, J = 4.7, 7.8 Hz, 1H), 5.77 (br. d, J = 2.4 Hz, 1H), 5.04 (br. d, J = 8.8 Hz, 1H), 4.88 (d, J = 13.7 Hz, 2H), 4.60 (d, J = 13.7 Hz, 2H), 3.68 (dd, J = 9.2, 14.9 Hz, 1H), 3.35 - 3.15 (m, 3H), 1.80 - 1.64 (m, 2H), 0.98 (t, J = 7.4 Hz, 3H); LCMS (ESI) m/z: 394.2 [M+H]⁺ | 107 |
| 2-(5-fluoro-2-pyridyl)-N-[(2S)-2-hydroxy-2-(3-pyridyl)ethyl]-N-propyl-acetamide | 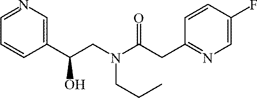 | ¹H NMR (400 MHz, Dimethylsulfoxide-d₆) δ 8.64 - 8.40 (m, 3H), 7.86 - 7.60 (m, 2H), 7.42 - 7.25 (m, 2H), 5.88 - 5.54 (m, 1H), 4.94 - 4.79 (m, 1H), 3.90 - 3.82 (m, 2H), 3.71 - 3.33 (m, 3H), 3.25 - 3.09 (m, 1H), 1.57 - 1.38 (m, 2H), 0.84 - 0.73 (m, 3H); LCMS (ESI) m/z: 318.1 [M+H]⁺ | 108 |
| 5,6-difluoro-N-[(2S)-2-hydroxy-2-(3-pyridyl)ethyl]-N-propyl-indane-2-carboxamide | 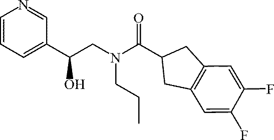 | ¹H NMR (400 MHz, Chloroform-d) δ 8.68 -8.49 (m, 2H), 7.77 (br. d, J = 7.7 Hz, 1H), 7.37 - 7.27 (m, 1H), 7.06 - 6.92 (m, 2H), 5.12 - 4.92 (m, 2H), 3.84 - 3.71 (m, 1H), 3.64 - 3.47 (m, 2H), 3.37 - 3.17 (m, 3H), 3.16 - 3.03 (m, 3H), 1.64 - 1.49 (m, 2H), 0.92 (t, J = 7.3 Hz, 3H); LCMS (ESI) m/z: 361.1 [M+H]⁺ | 110 |
| 2-(1,3-benzothiazol-7-yl)-N-[(2S)-2-hydroxy- | 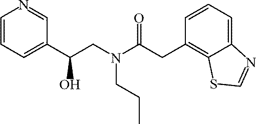 [SMILES: CCCN(C[C@@H](O)c1cccnc1)C(=O)Cc1cnc(C(F)(F)F)nc1] | ¹H NMR (400 MHz, Chloroform-d) δ 9.05 -8.95 (m, 1H), 8.63 - 8.46 (m, 2H), 8.13 -8.01 (m, 1H), 7.77 - 7.65 (m, 1H), 7.58 -7.44 (m, 1H), 7.38 - 7.29 (m, 1H), 7.24 (dd, | 118 |
| 2-(3-pyridyl)ethyl]-N-propyl-acetamide | | J = 4.8, 7.8 Hz, 1H), 5.04 (dd, J = 2.3, 8.1 Hz, 1H), 4.94 - 4.56 (m, 1H), 4.09 - 3.92 (m, 2H), 3.85 - 3.46 (m, 2H), 3.36 - 3.02 (m, 2H), 1.63 - 1.50 (m, 2H), 0.88 (t, J = 7.4 Hz, 3H); LCMS (ESI) m/z: 356.2 [M+H]⁺ | |
| 2-(4-cyano-3-fluoro-phenyl)-N-[(2S)-2-hydroxy-2-(3-pyridyl)ethyl]-N-propyl-acetamide | 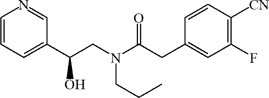 | ¹H NMR (400 MHz, Chloroform-d) δ 8.67 -8.52 (m, 2H), 7.79 - 7.72 (m, 1H), 7.66 -7.52 (m, 1H), 7.33 - 7.28 (m, 1H), 7.21 -7.09 (m, 2H), 5.12 - 4.95 (m, 1H), 4.47 -4.29 (m, 1H), 3.83 - 3.78 (m, 2H), 3.78 -3.68 (m, 1H), 3.59 - 3.49 (m, 1H), 3.38 -3.10 (m, 2H), 1.69 - 1.59 (m, 2H), 1.01 -0.86 (m, 3H); LCMS (ESI) m/z: 342.1 [M+H]⁺ | 119 |
| 4,5-dichloro-N-[(2S)-2-hydroxy-2-(3-pyridyl)ethyl]-N-propyl-indane-2-carboxamide | 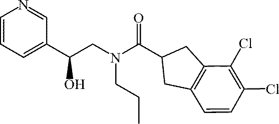 | ¹H NMR (400 MHz, Chloroform-d) δ 8.66 -8.58 (m, 1H), 8.58 - 8.52 (m, 1H), 7.82 -7.71 (m, 1H), 7.37 - 7.27 (m, 2H), 7.08 -6.97 (m, 1H), 5.13 - 4.98 (m, 1H), 4.98 -4.78 (m, 1H), 3.84 - 3.71 (m, 1H), 3.65 -3.51 (m, 2H), 3.49 - 3.23 (m, 4H), 3.23 -3.07 (m, 2H), 1.69 - 1.58 (m, 2H), 0.89 (s, 3H); LCMS (ESI) m/z: 393.1 [M+H]⁺ | 120 |
| N-[(2S)-2-hydroxy-2-(3-pyridyl)ethyl]-N-propyl-2-[2-(trifluoromethyl)pyri midin-5-yl]acetamide | 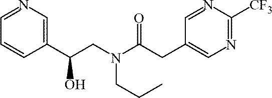 | ¹H NMR (400 MHz, Dimethylsulfoxide-d₆) δ 8.86 (d, J = 14.3 Hz, 2H), 8.71 - 8.52 (m, 1H), 8.52 - 8.41 (m, 1H), 7.93 - 7.70 (m, 1H), 7.47 - 7.31 (m, 1H), 5.98 - 5.55 (m, 1H), 4.98 - 4.65 (m, 1H), 3.76 - 3.49 (m, 1H), 3.48 - 3.28 (m, 4H), 3.30 - 3.13 (m, 1H), 1.68 - 1.44 (m, 2H), 0.93 - 0.77 (m, 3H); LCMS (ESI) m/z: 369.2 [M+H]⁺ | 124 |
| 2-(3,4-dichlorophenyl)-N-[2-hydroxy-2-[6-(trifluoromethyl)pyri dazin-3-yl]ethyl]-N-propyl-acetamide | 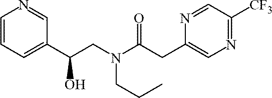 | ¹H NMR (400 MHz, Chloroform-d) δ 8.91 -8.82 (m, 1H), 8.77 - 8.69 (m, 1H), 8.65 -8.55 (m, 1H), 8.50 (dd, J = 1.4, 4.6 Hz, 1H), 7.79 - 7.73 (m, 1H), 7.27 (s, 1H), 5.11 - 4.99 (m, 1H), 4.78 - 4.25 (m, 1H), 4.12 -4.00 (m, 2H), 3.86 - 3.54 (m, 2H), 3.47 -3.09 (m, 2H), 1.72 - 1.63 (m, 2H), 0.96-0.87 (t, J = 7.4 Hz, 3H); LCMS (ESI) m/z: 369.1 [M+H]⁺ | 125 |
| N-[(2S)-2-hydroxy-2-(3-pyridyl)ethyl]-N-propyl-2-[2-(trifluoromethyl)-4-pyridyl]acetamide | 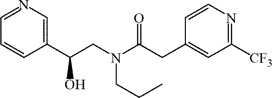 | ¹H NMR (400 MHz, Chloroform-d) δ 8.73 -8.68 (m, 1H), 8.62 - 8.58 (m, 1H), 8.54 (dd, J = 1.4, 4.8 Hz, 1H), 7.78 - 7.72 (m, 1H), 7.64 - 7.55 (m, 1H), 7.42 (d, J = 4.4 Hz, 1H), 7.32 - 7.27 (m, 1H), 5.10 - 4.97 (m, 1H), 4.37 - 4.24 (m, 1H), 3.82 (s, 2H), 3.73 (dd, J = 8.4, 14.2 Hz, 1H), 3.56 (dd, J = 2.8, 14.2 Hz, 1H), 3.33 - 3.17 (m, 2H), 1.64 - 1.60 (m, 2H), 0.95 (t, J = 7.4 Hz, 3H); LCMS (ESI) m/z: 368.1 [M+H]⁺ | 127 |
| N-[(2S)-2-hydroxy-2-(3-pyridyl)ethyl]-N-propyl-3-(trifluoromethyl)-6,7-dihydro-5H-cyclopenta[c]pyridin e-6-carboxamide | 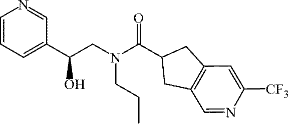 | ¹H NMR (400 MHz, Chloroform-d) δ 8.67 -8.48 (m, 3H), 7.82 - 7.69 (m, 1H), 7.61 -7.48 (m, 1H), 7.37 - 7.29 (m, 1H), 5.10 -4.95 (m, 1H), 4.76 (br. d, J = 14.1 Hz, 1H), 3.79 (ddd, J = 8.1, 11.7, 14.2 Hz, 1H), 3.69 - 3.08 (m, 8H), 1.67 - 1.57 (m, 2H), 0.94 (t, J = 7.3 Hz, 3H); ¹H NMR (400 MHz, Dimethylsulfoxide-d₆) δ 8.66 - 8.54 (m, 1H), 8.54 - 8.44 (m, 2H), 7.89 - 7.67 (m, 2H), 7.44 - 7.27 (m, 1H), 5.87 - 5.56 (m, 1H), 4.94 - 4.80 (m, 1H), 3.86 - 3.34 (m, 4H), 3.31 - 2.87 (m, 5H), 1.63 - 1.37 (m, 2H), 0.82 (td, J = 7.4, 10.7 Hz, 3H); LCMS (ESI) m/z: 394.2 [M+H]⁺ | 128 |
| 2-(2-tert-butylpyrimidin-5-yl)-N-[(2S)-2-hydroxy-2-(3-pyridyl)ethyl]-N-propyl-acetamide | 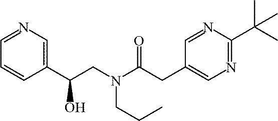 [SMILES: CCCN(C[C@@H](O)c1cccnc1)C(=O)C1Cc2cc(C#N)c(C#N)cc2C1] | ¹H NMR (400 MHz, Dimethylsulfoxide-d₆) δ 8.56 - 8.43 (m, 2H), 8.66 (d, J = 1.3 Hz, 1H), 7.90 - 7.68 (m, 1H), 7.41 (dd, J = 5.0, 7.6 Hz, 1H), 7.34 (dd, J = 4.9, 8.2 Hz, 1H), 6.00 - 5.51 (m, 1H), 5.01 - 4.74 (m, 1H), 3.87 - 3.64 (m, 2H), 3.52 (br. dd, J = 4.4, 13.7 Hz, 1H), 3.46 - 3.37 (m, 1H), 3.36 -3.33 (m, 2H), 1.65 - 1.43 (m, 2H), 1.35 (d, J = 4.6 Hz, 9H), 0.92 - 0.77 (m, 3H). 1H NMR (400 MHz, Chloroform-d) δ 8.68 -8.50 (m, 3H), 7.79 - 7.70 (m, 1H), 7.38 -7.27 (m, 1H), 5.08 - 4.98 (m, 1H), 4.52 (br. s, 1H), 3.91 - 3.59 (m, 3H), 3.54 (dd, J = 2.6, 14.4 Hz, 1H), 3.39 - 3.11 (m, 2H), 1.70 - 1.61 (m, 2H), 1.47 - 1.35 (m, 9H), 0.99 -0.88 (m, 3H); LCMS (ESI) m/z: 357.3 [M+H]⁺ | 130 |
| 5-bromo-6-chloro-N-[(2S)-2-hydroxy-2-(3-pyridyl)ethyl]-N-propyl-indane-2-carboxamide | 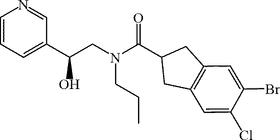 | ¹H NMR (400 MHz, Chloroform-d) δ 8.65 -8.51 (m, 2H), 7.80 - 7.74 (m, 1H), 7.49 -7.42 (m, 1H), 7.35 - 7.28 (m, 2H), 5.08 -5.01 (m, 1H), 5.01 - 4.93 (s, 1H), 3.83 -3.73 (m, 1H), 3.61 - 3.48 (m, 2H), 3.37 -3.16 (m, 3H), 3.15 - 3.02 (m, 3H), 1.62 -1.55 (m, 2H), 0.98 - 0.86 (t, 3H); LCMS (ESI) m/z: 437.1 [M+H]⁺ | 131 |
| N-[(2S)-2-hydroxy-2-(3-pyridyl)ethyl]-N-propyl-2-[6-(trifluoromethyl)-3-pyridyl]propanamid e | 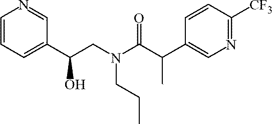 [SMILES: CCCN(C[C@@H](O)c1cccnc1)C(=O)N1Cc2cc(Cl)c(Br)cc2C1] | ¹H NMR (400 MHz, Methanol-d₄) δ 9.03 -8.86 (m, 1H), 8.84 - 8.57 (m, 3H), 8.19 -8.06 (m, 0.5H), 8.04 - 7.97 (m, 1H), 7.97 -7.91 (m, 0.5H), 7.82 - 7.76 (m, 1H), 5.25 -5.11 (m, 1H), 4.67 (d, J = 6.9 Hz, 0.4H), 4.38 - 4.28 (m, 1H), 3.86 (m, 0.6H), 3.75 -3.42 (m, 3H), 1.75 - 1.54 (m, 2H), 1.52 -1.40 (m, 3H), 0.98 - 0.84 (m, 3H); LCMS (ESI) m/z: 382.1 [M+H]⁺ | 132 |
| 5,6-dicyano-N-[(2S)-2-hydroxy-2-(3-pyridyl)ethyl]-N-propyl-indane-2-carboxamide | 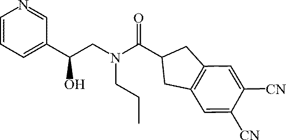 [SMILES: CC(C)(C)OC(=O)NCCCN(C[C@@H](O)c1cccnc1)C(=O)Cc1ccc(C(F)(F)F)nc1] | ¹H NMR (400 MHz, Chloroform-d) δ 8.65 -8.59 (m, 1H), 8.57 (dd, J = 1.6, 4.8 Hz, 1H), 7.79 - 7.72 (m, 1H), 7.67 - 7.61 (m, 2H), 7.35 - 7.29 (m, 1H), 5.10 - 4.96 (m, 1H), 4.55 - 4.44 (s, 1H), 3.83 - 3.72 (m, 1H), 3.70 - 3.60 (m, 1H), 3.56 (dd, J = 2.7, 14.2 Hz, 1H), 3.52 - 3.42 (m, 1H), 3.41 -3.21 (m, 4H), 3.21 - 3.11 (m, 1H), 1.71 -1.59 (m, 2H), 0.95 (t, J = 7.4 Hz, 3H); LCMS (ESI) m/z: 375.2 [M+H]⁺ | 133 |
| 5-chloro-6-cyano-N-[(2S)-2-hydroxy-2-(3-pyridyl)ethyl]-N-propyl-indane-2-carboxamide | 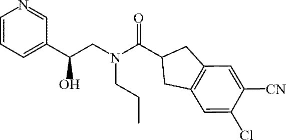 [SMILES: CC(C)(C)OC(=O)NCCCCCN(C[C@@H](O)c1cccnc1)C(=O)Cc1ccc(C(F)(F)F)nc1] | ¹H NMR (400 MHz, Chloroform-d) δ 8.69 -8.49 (m, 2H), 7.82 - 7.71 (m, 1H), 7.53 -7.45 (m, 1H), 7.38 - 7.35 (m, 1H), 7.34 -7.28 (m, 1H), 5.09 - 4.95 (m, 1H), 4.80 -4.59 (s, 1H), 3.83 - 3.72 (m, 1H), 3.66 -3.52 (m, 2H), 3.48 - 3.09 (m, 6H), 1.66 -1.58 (m, 2H), 0.98 - 0.89 (t, 3H); LCMS (ESI) m/z: 384.2 [M+H]⁺ | 134 |
| 5-bromo-6-chloro-N-[(2S)-2-hydroxy-2-(3-pyridyl)ethyl]-N-propyl-isoindoline-2-carboxamide | 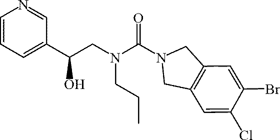 [SMILES: CC(C)(C)OC(=O)NCCCCCN(C[C@@H](O)c1cccnc1)C(=O)Cc1ccc(C#N)c(Cl)c1] | ¹H NMR (400 MHz, Chloroform-d) δ 8.61 (s, 1H), 8.54 (br. d, J = 4.0 Hz, 1H), 7.82 (br. d, J = 7.8 Hz, 1H), 7.54 (s, 1H), 7.38 (s, 1H), 7.30 (dd, J = 5.0, 7.9 Hz, 1H), 5.77 - 5.57 (m, 1H), 5.05-5.02 (m, 1H), 4.87-4.84 (m, 2H), 4.62-4.56 (m, 2H), 3.68 (dd, J = 9.1, 14.7 Hz, 1H), 3.36 - 3.25 (m, 2H), | 135 |
| | | 3.25 - 3.15 (m, 1H), 1.83 - 1.66 (m, 2H), 0.98 (t, J = 7.4 Hz, 3H); LCMS (ESI) m/z: 438.1 [M+H]⁺ | |
| tert-butyl N-[3-[[(2S)-2-hydroxy-2-(3-pyridyl)ethyl]-[2-[6-(trifluoromethyl)-3-pyridyl]acetyl]amino ]propyl]carbamate | 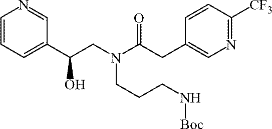 [SMILES: CC(C)(C)OC(=O)NCCN(C[C@@H](O)c1cccnc1)C(=O)Cc1ccc(C(F)(F)F)nc1] | ¹H NMR (400 MHz, CHLOROFORM-d) δ 8.68 - 8.48 (m, 3H), 7.87 - 7.60 (m, 3H), 7.40 - 7.28 (m, 1H), 5.10 - 5.02 (m, 1H), 4.80 - 4.71 (m, 1H), 4.40 (br s, 1H), 4.13 (br d, J=15.9 Hz, 1H), 3.86 - 3.70 (m, 2H), 3.65 - 3.53 (m, 2H), 3.49 - 3.32 (m, 2H), 3.21 - 3.06 (m, 2H), 1.81 (dt, J=6.5, 13.6 Hz, 2H), 1.42 (d, J=8.8 Hz, 9H) LCMS (ESI) m/z: 483.3 [M+H]+ | 140 |
| tert-butyl N-[5-[[(2S)-2-hydroxy-2-(3-pyridyl)ethyl]-[2-[6-(trifluoromethyl)-3-pyridyl]acetyl]amino ]pentyl]carbamate | 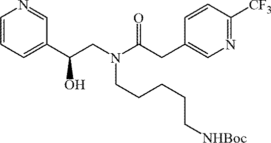 [SMILES: CC(C)(C)OC(=O)NCCCCN(C[C@@H](O)c1cccnc1)C(=O)Cc1ccc(C(F)(F)F)nc1] | ¹H NMR (400 MHz, CHLOROFORM-d) δ 8.67 - 8.50 (m, 3H), 7.86 - 7.63 (m, 3H), 7.38 - 7.28 (m, 1H), 5.04 (br dd, J = 2.1, 8.3 Hz, 1H), 4.65 - 4.54 (m, 1H), 3.82 (s, 1H), 3.75 - 3.52 (m, 3H), 3.42 - 3.20 (m, 3H), 3.17 - 3.07 (m, 2H), 1.54 (br s, 4H), 1.43 (s, 9H), 1.37 - 1.30 (m, 2H); LCMS (ESI) m/z: 511.2 [M+H]+ | 141 |
| 1-[4-[3,5-dichloro-4-[(2-ethoxy-3-pyridyl)methoxy]ph enyl]-2-pyridyl]piperazine | 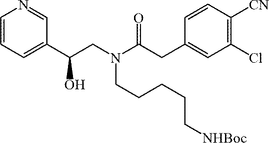 | ¹H NMR (400 MHz, CHLOROFORM-d) δ 8.67 - 8.51 (m, 2H), 7.75 (br d, J = 7.5 Hz, 1H), 7.66 (d, J = 7.9 Hz, 1H), 7.46 (s, 1H), 7.39 - 7.28 (m, 2H), 5.04 (br dd, J = 1.8, 7.7 Hz, 1H), 4.58 (dt, J = 1.7, 2.7 Hz, 2H), 3.79 (s, 1H), 3.72 - 3.64 (m, 1H), 3.61 -3.56 (m, 1H), 3.37 - 3.08 (m, 5H), 1.54 -1.48 (m, 2H), 1.45 - 1.40 (m, 9H), 1.39 -1.21 (m, 4H); LCMS (ESI) m/z :501.2 [M+H]+ | 142 |
| tert-butyl N-[2-[[(2S)-2-hydroxy-2-(3-pyridyl)ethyl]-[2-[6-(trifluoromethyl)-3-pyridyl]acetyl]amino ]ethyl]carbamate | 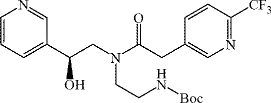 | ¹H NMR (399 MHz, DMSO-d₆, T=273+80K) δ 8.72 - 8.40 (m, 3H), 8.03 - 7.61 (m, 3H), 7.47 - 7.23 (m, 1H), 6.85 - 6.40 (m, 1H), 5.79 - 5.18 (m, 1H), 5.00 - 4.81 (m, 1H), 4.11 - 3.77 (m, 2H), 3.39 (br s, 2H), 3.25 -3.09 (m, 2H), 1.40 - 1.34 (s, 9H); LCMS (ESI) m/z: 469.1 [M+H]+ | 143 |
| (S)-tert-butyl (4-(N-(2-hydroxy-2-(pyridin-3-yl)ethyl)-2-(6-(trifluoromethyl)pyri | 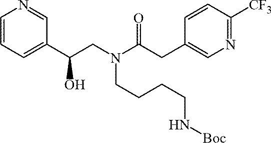 [SMILES: CCCN(C[C@H](O)c1cccnc1)C(=O)Cc1ccc2ccccc2c1] | ¹H NMR (400 MHz, METHANOL-d₄) δ 8.71 - 8.50 (br d, J=9.9 Hz, 3H), 8.03 - 7.90 (m, 1H), 7.87 (br d, J=7.9 Hz, 1H), 7.81 - 7.73 (m, 1H), 7.52 - 7.35 (m, 1H), 5.04 (dt, J=3.9, 8.4 Hz, 1H), 4.70 -4.66 (m, 1), 4.65- | 144 |
| din-3-yl)acetamido)butyl) carbamate | | 4.52 (m, 1H), 4.03 - 3.78 (m, 2H), 3.60 -3.35 (m, 2H), 3.34 - 3.33 (m, 2H) 3.14 -3.00 (m, 2H), 1.74 - 1.59 (m, 2H), 1.56 -1.45 (m, 2H), 1.41 (s, 9H); LCMS (ESI) m/z: 497.2 [M+H]+ | |
| tert-butyl N-[4-[[[(2S)-2-hydroxy-2-(3-pyridyl)ethyl]-[2-[6-(trifluoromethyl)-3-pyridyl]acetyl]amino ]methyl]cyclohexyl] carbamate | 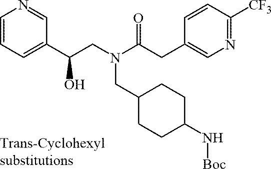 | ¹H NMR (399 MHz, DMSO-d₆, T=273+80K) δ 8.58 - 8.45 (m, 1H), 8.45 - 8.44 (m,2 H), 7.91 - 7.66 (m, 3H), 7.46 - 7.20 (m, 1H), 6.40 - 6.06 (m, 1H), 5.78 - 5.58 (m, 1H), 5.48 - 5.29 (m, 1H), 4.96 - 4.86 (m, 1H), 4.08 - 3.78 (m, 2H), 3.77 - 3.53 (m, 1H), 3.52 - 3.28 (m, 2H), 3.27 - 3.08 (m, 2H), 1.90 - 1.72 (m, 2H), 1.62 (br s, 3H), 1.39 (s, 9H), 1.24 - 0.86 (m, 4H); LCMS (ESI) m/z: 537.3 [M+H}+ | 145 |
| tert-butyl N-[4-[[[(2S)-2-hydroxy-2-(3-pyridyl)ethyl]-[2-[6-(trifluoromethyl)-3-pyridyl]acetyl]amino ]methyl]cyclohexyl] carbamate | 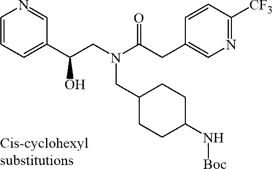 | ¹H NMR (400 MHz, CHLOROFORM-d) δ 8.72 - 8.51 (m, 3H), 7.90 - 7.59 (m, 3H), 7.40 - 7.28 (m, 1H), 5.09 - 4.97 (m, 1H), 4.70 - 4.51 (m, 1H), 4.32 - 3.96 (m, 1H), 3.89 - 3.54 (m, 5H), 3.45 - 3.02 (m, 2H), 1.86 - 1.60 (m, 7H), 1.44 (s, 9H), 1.31 -1.07 (m, 2H); LCMS (ESI) m/z: 537.3 [M+H]+ | 146 |
| N-[(2R)-2-hydroxy-2-(3-pyridyl)ethyl]-2-(2-naphthyl)-N-propyl-acetamide | 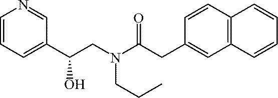 | ¹H NMR (400 MHz, CHLOROFORM-d) δ 8.58 (d, J=1.7 Hz, 1H), 8.50 (dd, J=1.5, 4.8 Hz, 1H), 7.89 - 7.78 (m, 3H), 7.75 - 7.69 (m, 2H), 7.54 - 7.38 (m, 3H), 7.18 (dd, J=4.8, 7.8 Hz, 1H), 5.10 (br s, 1 H), 5.03 (br d, J=6.0 Hz, 1H), 3.92 (s, 2H), 3.80 (dd, J=7.9, 14.3 Hz, 1H), 3.53 (dd, J=2.2, 14.4 Hz, 1H), 3.29 (ddd, J=6.6, 8.8, 15.0 Hz, 1H), 3.07 (ddd, J=6.4, 9.0, 14.9 Hz, 1H), 1.57 - 1.48 (m, 2H), 0.87 (t, J=7.4 Hz, 3H); LCMS (ESI) m/z: 349.1 [M+H]+ | 147 |

### Example 5: Preparation of 1-(3,4-Dichlorophenyl)-N-[2-Hydroxy-2-(3-Pyridyl)ethyl]-N-Propyl-Methanesulfonamide (Compound 10)

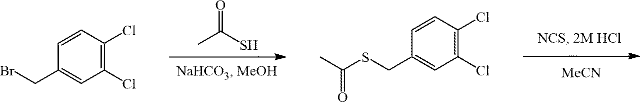

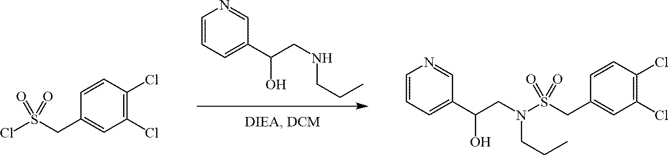

### Step 1: Preparation of S-[(3,4-Dichlorophenyl)Methyl] Ethanethioate

To a stirred solution of 4-(bromomethyl)-1,2-dichloro-benzene (1.20 g, 5.00 mmol) in methanol (10 mL) were added ethanethioic acid (457 mg, 6.00 mmol) and sodium bicarbonate (504 mg, 6.00 mmol) at 20° C. The mixture was stirred at 20° C. for 15 h and the mixture was poured into water (20 mL). The aqueous phase was extracted with ethyl acetate (20 mL × 3). The combined organic phase were dried with anhydrous sodium sulfate, filtered and concentrated in vacuo to give S-[(3,4-dichlorophenyl)methyl] ethanethioate (1 g, 4.25 mmol, 85%) as a yellow oil.

### Step 2: Preparation of (3,4-Dichlorophenyl)Methanesulfonyl Chloride

A solution of NCS (1.14 g, 8.51 mmol) in acetonitrile (5 mL) and HCl (12 M, 177 µL) was cooled to 0° C., and then a solution of S-[(3,4-dichlorophenyl)methyl] ethanethioate (0.50 g, 2.13 mmol) in acetonitrile (1 mL) was added dropwise. The mixture was stirred at 0° C. for 0.5 h and then poured into water (20 mL). The aqueous phase was extracted with ethyl acetate (20 mL × 3). The combined organic phases were dried with anhydrous sodium sulfate, filtered and concentrated in vacuo to give (3,4-dichlorophenyl)methanesulfonyl chloride (0.7 g, crude) as a white solid.

### Step 3: Preparation of 1-(3,4-Dichlorophenyl)-N-[2-Hydroxy-2-(3-Pyridyl)ethyl]-N-Propyl-Methanesulfonamide

To a stirred solution of (3,4-dichlorophenyl)methanesulfonyl chloride (200 mg, 771 µmol) in dichloromethane (2 mL) at 0° C. were added 2-(propylamino)-1-(3-pyridyl)ethanol (139 mg, 771 µmol) and diisopropylethylamine (199 mg, 1.54 mmol, 268 µL). The mixture was stirred at 0° C. for 0.5 h and concentrated in vacuum. The resultant crude product purified by prep-HPLC (Waters Xbridge Prep OBD C18 150 × 40 mm × 10 uM column; 40-60% acetonitrile in a 10 mM ammonium bicarbonate solution in water, 10 min gradient) to give 1-(3,4-dichlorophenyl)-N-[2-hydroxy-2-(3-pyridyl)ethyl]-N-propyl-methanesulfonamide (32 mg, 77 µmol, 10%) as a pale yellow thick oil. ¹H NMR (400 MHz, Dimethylsulfoxide-d₆) δ 8.60 - 8.55 (m, 1H), 8.49 (d, J = 4.6 Hz, 1H), 7.81 - 7.75 (m, 1H), 7.69 (s, 1H), 7.66 (d, J = 8.2 Hz, 1H), 7.44 - 7.32 (m, 2H), 5.82 - 5.75 (m, 1H), 4.88 - 4.80 (m, 1H), 4.54 - 4.43 (m, 2H), 3.40 - 3.33 (m, 1H), 3.30 - 3.25 (m, 1H), 3.20 - 2.98 (m, 2H), 1.54 - 1.39 (m, 2H), 0.79 - 0.71 (m, 3H); LCMS (ESI) m/z: 403.1 [M+H]⁺.

### Example 6: Preparation of 1-(3,4-Dichlorophenyl)-N-[2-Hydroxy-2-(3-Pyridyl)ethyl]-N-Propyl-Methanesulfonamide (Compound 10)

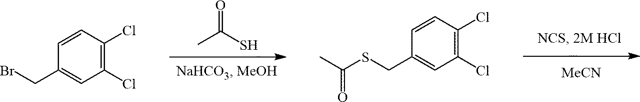

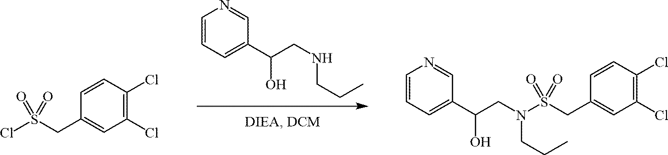

### Step 1: Preparation of S-[(3,4-Dichlorophenyl)methyl] Ethanethioate

To a stirred solution of 4-(bromomethyl)-1,2-dichloro-benzene (1.20 g, 5.00 mmol) in methanol (10 mL) were added ethanethioic acid (457 mg, 6.00 mmol) and sodium bicarbonate (504 mg, 6.00 mmol) at 20° C. The mixture was stirred at 20° C. for 15 h and the mixture was poured into water (20 mL). The aqueous phase was extracted with ethyl acetate (20 mL × 3). The combined organic phase were dried with anhydrous sodium sulfate, filtered and concentrated in vacuo to give S-[(3,4-dichlorophenyl)methyl] ethanethioate (1 g, 4.25 mmol, 85%) as a yellow oil.

### Step 2: Preparation of (3,4-Dichlorophenyl)Methanesulfonyl Chloride

A solution of NCS (1.14 g, 8.51 mmol) in acetonitrile (5 mL) and HCl (12 M, 177 µL) was cooled to 0° C., and then a solution of S-[(3,4-dichlorophenyl)methyl] ethanethioate (0.50 g, 2.13 mmol) in acetonitrile (1 mL) was added dropwise. The mixture was stirred at 0° C. for 0.5 h and then poured into water (20 mL). The aqueous phase was extracted with ethyl acetate (20 mL × 3). The combined organic phases were dried with anhydrous sodium sulfate, filtered and concentrated in vacuo to give (3,4-dichlorophenyl)methanesulfonyl chloride (0.7 g, crude) as a white solid.

### Step 3: Preparation of 1-(3,4-Dichlorophenyl)-N-[2-Hydroxy-2-(3-Pyridyl)ethyl]-N-Propyl-Methanesulfonamide.

To a stirred solution of (3,4-dichlorophenyl)methanesulfonyl chloride (200 mg, 771 µmol) in dichloromethane (2 mL) at 0° C. were added 2-(propylamino)-1-(3-pyridyl)ethanol (139 mg, 771 µmol) and diisopropylethylamine (199 mg, 1.54 mmol, 268 µL). The mixture was stirred at 0° C. for 0.5 h and concentrated in vacuum. The resultant crude product purified by prep-HPLC (Waters Xbridge Prep OBD C18 150 × 40 mm × 10 uM column; 40-60% acetonitrile in a 10 mM ammonium bicarbonate solution in water, 10 min gradient) to give 1-(3,4-dichlorophenyl)-N-[2-hydroxy-2-(3-pyridyl)ethyl]-N-propyl-methanesulfonamide (32 mg, 77 µmol, 10%) as a pale yellow thick oil. ¹H NMR (400 MHz, Dimethylsulfoxide-d₆) δ 8.60 - 8.55 (m, 1H), 8.49 (d, J = 4.6 Hz, 1H), 7.81 - 7.75 (m, 1H), 7.69 (s, 1H), 7.66 (d, J = 8.2 Hz, 1H), 7.44 - 7.32 (m, 2H), 5.82 - 5.75 (m, 1H), 4.88 - 4.80 (m, 1H), 4.54 - 4.43 (m, 2H), 3.40 - 3.33 (m, 1H), 3.30 - 3.25 (m, 1H), 3.20 - 2.98 (m, 2H), 1.54 - 1.39 (m, 2H), 0.79 - 0.71 (m, 3H); LCMS (ESI) m/z: 403.1 [M+H]⁺.

### Example 7: Preparation of 2-(3,4-Dichlorophenyl)-N-(2-Hydroxy-2-Pyrimidin-5-yl-Ethyl)-N-Propyl-Acetamide (Compound 13)

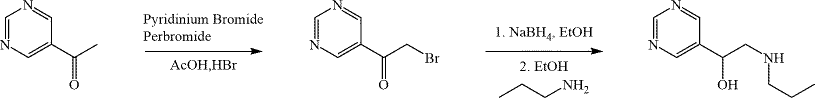

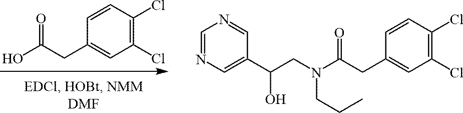 [SMILES: CCCN(C[C@@H](O)c1cccnc1)C(=O)Cc1ccc(C)nc1]

### Step 1: Preparation of 2-Bromo-1-Pyrimidin-5-yl-Ethanone

To a solution of 1-pyrimidin-5-ylethanone (1 g, 8.19 mmol) in HBr (2 mL) and acetic acid (10 mL) was added pyridinium tribromide (2.64 g, 8.27 mmol). The reaction mixture was stirred at 15° C. for 12 h and filtered. The resultant solids were dried in vacuo to afford crude 2-bromo-1-pyrimidin-5-yl-ethanone.HBr (1.5 g, crude) as a pale yellow solid and which was used in the next step without further purification.

### Step 2: Preparation of 2-(Propylamino)-1-Pyrimidin-5-yl-Ethanol

To a solution of 2-bromo-1-pyrimidin-5-yl-ethanone.HBr (800 mg, 3.06 mmol) in ethanol (10 mL) was added sodium borohydride (348 mg, 9.19 mmol) at 0° C. Then the mixture was stirred at 15° C. for 2 h and filtered. The resultant filtrate was treated with propan-1-amine (543 mg, 9.19 mmol) and the entire reaction mixture was stirred at 80° C. for 12 h. Concentration and purification by prep-HPLC (Waters Xbridge BEH C18 100 × 30 mm × 10 um column; 1-10% acetonitrile in a 0.1% trifluoroacetic acid solution in water, 10 min gradient) (acid) afforded 2-(propylamino)-1-pyrimidin-5-yl-ethanol.TFA (30 mg, 92 µmol, 3%) as a pale yellow solid. ¹H NMR (400 MHz, Methanol-d₄) δ 9.25 (s, 1H), 9.02 - 8.97 (m, 2H), 4.54 -4.44 (m, 1H), 4.15 (dd, J = 3.8, 12.0 Hz, 1H), 3.89 (dd, J = 3.8, 12.0 Hz, 1H), 3.13 - 2.99 (m, 1H), 2.97 -2.80 (m, 1H), 1.84 - 1.67 (m, 2H), 1.07 - 0.93 (m, 3H); LCMS (ESI) m/z: 182.1 [M+H]⁺.

### Step 3: 2-(3,4-Dichlorophenyl)-N-(2-Hydroxy-2-Pyrimidin-5-yl-Ethyl)-N-Propyl-Acetamide

To a solution of 2-(3,4-dichlorophenyl)acetic acid (21 mg, 102 µmol) in dimethylformamide (0.5 mL) were added N-(3-dimethylaminopropyl)-N′-ethylcarbodiimide (23 mg, 122 µmol), 1-hydroxybenzotriazole (17 mg, 122 µmol), N-methylmorpholine (31 mg, 305 µmol, 34 µL) and 2-(propylamino)-1-pyrimidin-5-yl-ethanol.TFA (30 mg, 1021 µmol). The mixture was stirred at 15° C. for 2 h and concentrated. The resultant crude product purified by prep-HPLC (Waters Xbridge BEH C18 100 × 25 mm × 5 um column; 25-55% acetonitrile in a 10 mM ammonium bicarbonate solution in water, 8 min gradient) (neutral) to give 2-(3,4-dichlorophenyl)-N-(2-hydroxy-2-pyrimidin-5-yl-ethyl)-N-propyl-acetamide (7 mg, 19 µmol, 19%) as a white solid. ¹H NMR (400 MHz, Chloroform-d) δ 9.16 (s, 1H), 8.71 (s, 2H), 7.43 (d, J = 8.2 Hz, 1H), 7.36 (d, J = 1.5 Hz, 1H), 7.11 (dd, J = 1.6, 8.1 Hz, 1H), 4.95 - 4.87 (m, 1H), 4.34 - 4.17 (m, 2H), 3.73 (s, 2H), 3.37 - 3.16 (m, 2H), 3.12 - 2.95 (m, 1H), 1.66 - 1.58 (m, 2H), 0.92 (t, J = 7.3 Hz, 3H); LCMS (ESI) m/z: 368.1 [M+H]⁺.

### Example 8: Preparation of 2-(5-Chloro-2-Pyridyl)-N-[(2S)-2-Hydroxy-2-(3-Pyridyl)Ethyl]-N-Propyl-Acetamide (Compound 14)

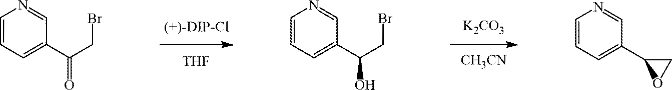

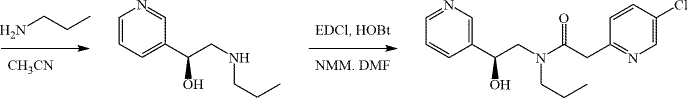

### Step 1: Preparation of (1S)-2-Bromo-1-(3-Pyridyl)Ethanol

To a solution of 2-bromo-1-(pyridin-3-yl)ethan-1-one.HBr (10 g, 35.59 mmol) in tetrahydrofuran (180 mL) was added chloro-bis[(1R,2R,3S,5R)-2,6,6-trimethylnorpinan-3-yl]borane (1.7 M, 104.69 mL) in heptane and triethylamine (3.96 g, 39.15 mmol) at -20° C. and the mixture was stirred at -20° C. for 2 h. Then the mixture was warmed to 20° C. and stirred for an additional 36 h. The reaction solution was basified with triethylamine to pH= 8-9 at 0° C. and then warmed to room temperature (20° C.) and poured into water (100 mL). The layers were separated, and the aqueous phase was extracted with ethyl acetate (50 mL × 4). The combined organic phases were washed with brine (30 mL), dried over anhydrous sodium sulfate and concentrated. The crude product was purified by flash column (ISCO 220 g silica, 0-100% ethyl acetate and 0.2% triethylamine in petroleum ether, gradient over 40 min) to give (1S)-2-bromo-1-(3-pyridyl)ethanol (11 g, crude) as brown viscous liquid. ¹H NMR (400 MHz, Dimethylsulfoxide-d₆) δ 8.59 (s, 1H), 8.48 (d, J = 4.4 Hz, 1H), 7.80 (br. d, J = 7.8 Hz, 1H), 7.41 - 7.33 (m, 1H), 5.97 (d, J = 4.8 Hz, 1H), 4.88 (q, J = 5.1 Hz, 1H), 3.76 - 3.70 (m, 1H), 3.69 - 3.62 (m, 1H); LCMS (ESI) m/z: 202.0 [M+H]⁺.

### Step 2: Preparation of 3-[(2S)-Oxiran-2-yl]Pyridine

To a solution of (1S)-2-bromo-1-(3-pyridyl)ethanol (4 g, 19.80 mmol) in acetonitrile (10 mL) was added potassium carbonate (10.94 g, 79.19 mmol). The mixture was stirred at 80° C. for 3 h, cooled and filtered. The filtrate containing the crude product 3-[(2S)-oxiran-2-yl]pyridine (2.4 g, crude) in acetonitrile as a brown solution was used in next step without further purification.

### Steps 3 and 4 Were Performed Similar to the Protocol Described for Compound 7

The product 2-(5-chloro-2-pyridyl)-N-[(2S)-2-hydroxy-2-(3-pyridyl)ethyl]-N-propyl-acetamide was obtained as a pale yellow solid. ¹H NMR (400 MHz, Chloroform-d) δ 8.68 - 8.57 (m, 1H), 8.56 - 8.46 (m, 2H), 7.81 - 7.72 (m, 1H), 7.68 (dd, J = 2.6, 8.3 Hz, 1H), 7.36 - 7.31 (m, 1H), 7.28 - 7.26 (m, 1H), 5.14 -5.03 (m, 1H), 4.96 (d, J = 3.4 Hz, 1H), 4.24 - 3.87 (m, 2H), 3.80 - 3.55 (m, 2H), 3.52 - 3.32 (m, 1H), 3.27 - 3.13 (m, 1H), 1.61 - 1.50 (m, 2H), 0.95 - 0.87 (m, 3H); LCMS (ESI) m/z: 334.2 [M+H]⁺.

Compound 15 was synthesized according to the protocol described for the Compound 14.

| Name | Structure | NMR, MS | Compd# |
|---|---|---|---|
| N-[(2S)-2-hydroxy-2-(3-pyridyl)ethyl]-2-(6-methyl-3-pyridyl)-N-propyl-acetamide | 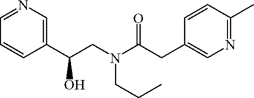 | ¹H NMR (400 MHz, Methanol-d₄) δ 8.66 - 8.53 (m, 1H), 8.52 - 8.42 (m, 1H), 8.31 - 8.19 (m, 1H), 8.00 - 7.82 (m, 1H), 7.63 - 7.52 (m, 1H), 7.50 - 7.37 (m, 1H), 7.30 - 7.22 (m, 1H), 5.02 (br. dd, J = 4.2, 7.7 Hz, 1H), 3.95 - 3.65 (m, 3H), 3.60 - 3.23 (m, 3H), 2.51 (d, J = 8.3 Hz, 3H), 1.70 - 1.54 (m, 2H), 0.91 (td, J = 7.4, 12.5 Hz, 3H); LCMS (ESI) m/z: 314.3 [M+H]⁺. | 15 |

### Example 9: Preparation of 2-(3-Chloro-4-Methyl-Phenyl)-N-[(2S)-2-Hydroxy-2-(3-Pyridyl)ethyl]-N-Propyl-Acetamide (Compound 16)

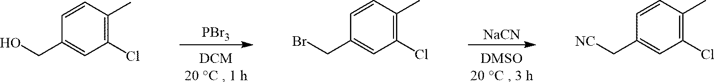 [SMILES: CC(C)N(C[C@@H](O)c1cccnc1)C(=O)Cc1ccc(Cl)c(Cl)c1]

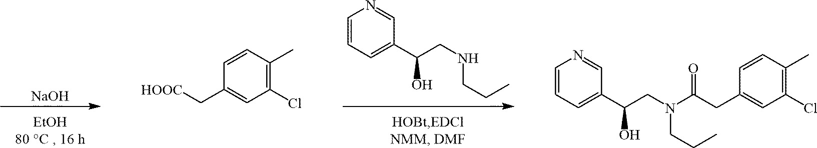 [SMILES: CCN(C[C@@H](O)c1cccnc1)C(=O)Cc1ccc(Cl)c(Cl)c1]

### Step 1: Preparation of 4-(bromomethyl)-2-chloro-1-methyl-benzene.

To a solution of (3-chloro-4-methyl-phenyl)methanol (1.00 g, 6 mmol) in dichloromethane (15 mL) was added PBr₃ (1.90 g, 7.02 mmol). The reaction mixture was stirred at 20° C. for 1 h. The mixture was then cooled to 0° C. and quenched by the slow addition of methanol (10 mL), further diluted with water (10 mL) and extracted with dichloromethane (10 mL × 2). The combined organic layer was concentrated to dryness to give 4-(bromomethyl)-2-chloro-1-methyl-benzene (1.10 g, 5.01 mmol, 78%) as a yellow oil. The material was used directly in the next step without additional purification.

### Step 2: Preparation of 2-(3-chloro-4-methyl-phenyl)acetonitrile.

To a solution of 4-(bromomethyl)-2-chloro-1-methyl-benzene (1.00 g, 4.56 mmol) in dimethylsulfoxide (1 mL) was added sodium cyanide (447 mg, 9 mmol) at 0° C. The reaction mixture was then stirred at 25° C. for 3 h. Water (20 mL) was added to the reaction and the aqueous phase was extracted with ethyl acetate (20 mL × 2). The combined organic layers were washed with brine (20 mL), dried over sodium sulfate, filtered and concentrated. The crude 2-(3-chloro-4-methyl-phenyl)acetonitrile (530 mg, 3.20 mmol, 70%) was obtained as a brown thick oil and used directly in the next step. ¹H NMR (400 MHz, Chloroform-d) δ 7.33 (s, 1H), 7.24 (d, J = 7.9 Hz, 1H), 7.13 (dd, J = 1.3, 7.9 Hz, 1H), 3.71 (s, 2H), 2.38 (s, 3H)

### Step 3: Preparation of 2-(3-Chloro-4-Methyl-Phenyl)Acetic Acid

To a solution of 2-(3-chloro-4-methyl-phenyl)acetonitrile (300 mg, 1.81 mmol) in ethanol (3 mL) was added sodium hydroxide (2 M, 1.81 mL) under N₂. Then the mixture was stirred at 80° C. for 12 h. Water (5 mL) was added to the reaction and the aqueous phase was extracted with ethyl acetate (30 mL × 2). The aqueous phase was acidified with 1 M HCl to pH=3~4 at 0° C. and then extracted with ethyl acetate (30 mL × 2). The combined organic layers were washed with brine (50 mL), dried over sodium sulfate, filtered and concentrated. The crude 2-(3-chloro-4-methyl-phenyl)acetic acid (263 mg, 1.42 mmol, 79%) was obtained as a pale yellow solid and used directly in the next step without further purification.

### Step 4: Preparation of 2-(3-Chloro-4-Methyl-Phenyl)-N-[(2S)-2-Hydroxy-2-(3-Pyridyl)ethyl]-N-Propyl-Acetamide

To a solution of 2-(3-chloro-4-methyl-phenyl)acetic acid (101 mg, 549 µmol) in dimethylformamide (2 mL) was added 1-hydroxybenzotriazole (81 mg, 599 µmol), N-(3-dimethylaminopropyl)-N′-ethylcarbodiimide (115 mg, 599 µmol), N-methylmorpholine (152 mg, 1.50 mmol, 165 µL) and (1S)-2-(propylamino)-1-(3-pyridyl)ethanol (90 mg, 499 µmol). The mixture was stirred at 20° C. for 2 h. The reaction mixture was concentrated in vacuo. The crude material was purified by prep-HPLC (Waters Xbridge BEH C18 100 × 30 mm × 10 um column; 30-60% acetonitrile in an a 10 mM ammonium bicarbonate solution in water, 10 min gradient) to give 2-(3-chloro-4-methyl-phenyl)-N-[(2S)-2-hydroxy-2-(3-pyridyl)ethyl]-N-propyl-acetamide (13 mg, 38 µmol, 8%) as a pale yellow solid. ¹H NMR (400 MHz, Chloroform-d) δ 8.64 - 8.50 (m, 2H), 7.76 (br. d, J = 7.7 Hz, 1H), 7.38 - 7.27 (m, 2H), 7.23 (br. d, J = 7.7 Hz, 1H), 7.08 (br. d, J = 7.7 Hz, 1H), 5.05 (br. d, J = 7.1 Hz, 2H), 3.81 - 3.72 (m, 1H), 3.70 (s, 2H), 3.53 (br. d, J = 14.3 Hz, 1H), 3.24 (br. dd, J = 6.9, 15.3 Hz, 1H), 3.14 - 3.03 (m, 1H), 2.39 (s, 3H), 1.62 - 1.49 (m, 2H), 0.91 (br. t, J = 7.4 Hz, 3H); LCMS (ESI) m/z: 347.0 [M+H]⁺.

### Example 10: Preparation of N-[(2S)-2-Hydroxy-2-(3-Pyridyl)Ethyl]-N-Propyl-2-[6-(Trifluoromethyl)-3-Pyridyl]Acetamide (Compound 17)

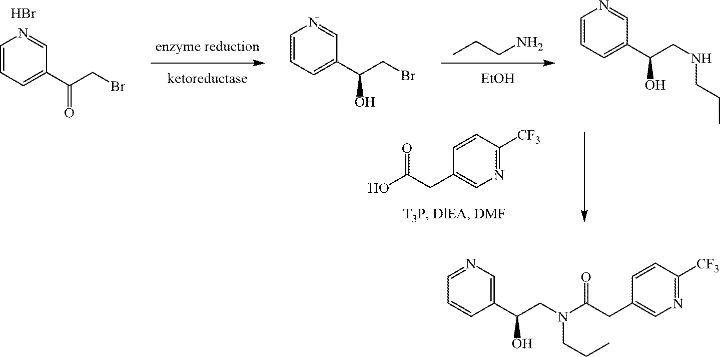

### Step 1: (1S)-2-Bromo-1-(3-Pyridyl)Ethanol

To a solution of (2R,3S,4R,5R)-2,3,4,5,6-pentahydroxyhexanal;hydrate (6.35 g, 32.03 mmol) in BUFFER (90 mL) were added NAD (78 mg, 10.68 mmol), NADP (78 mg, 10.68 mmol), ketoreductase (900 mg, 10.68 mmol) and GDH (156 mg, 10.68 mmol). Then 2-bromo-1-(3-pyridyl)ethanone;hydrobromide (3 g, 10.68 mmol) in BUFFER (60 mL) was added to the mixture. Then the mixture was stirred at 35° C. for 10 min, then was added 1 N NaOH to PH=7. After 0.5 h interval, 1 N NaOH was used to adjust the pH =7 until the pH did not change, then the mixture was stirred at 30° C. for 12 h. The resultant mixture was filtered and the solid was rinsed with EtOAc (50 mL * 3). The combined filtrates were extracted first with EtOAc and THF (2:1) (100 mL * 4) and then with CHCl₃/i-PrOH (50 mL*3). The combined organic layers were dried over Na₂SO₄, filtered and concentrated under reduced pressure. The compound (1S)-2-bromo-1-(3-pyridyl)ethanol (0.6 g, 2.82 mmol) was obtained as a pale yellow gum which was used in next step directly. LCMS (ESI) m/z: 202.9 [M+H]⁺.

Note: Buffer: A mixture of NaH₂PO4.2H₂O (3.96 g) and Na₂HPO₄.12H₂O (11.1 g) were dissolved in H₂O (500 mL) to make 0.1 M (pH = 7) aqueous solution.

### Step 2: (1S)-2-(Propylamino)-1-(3-Pyridyl)Ethanol

To a solution of (1S)-2-bromo-1-(3-pyridyl)ethanol (600 mg, 2.97 mmol) in EtOH (10 mL) was added propan-1-amine (4.31 g, 72.98 mmol) and the mixture was stirred at 80° C. for 2 h. The reaction mixture was concentrated and was purified by prep-HPLC (Waters Xbridge Prep OBD C18 150*40 mm*10 um column; 1-35% acetonitrile in an a 10 mM ammonium bicarbonate solution in water and in an a 0.05% ammonia solution in water, 8 min gradient) to afford (1S)-2-(propylamino)-1-(3-pyridyl)ethanol (300 mg, 1.33 mmol, 45%) as a yellow gum. LCMS (ESI) m/z: 181.2 [M+H]⁺.

¹H NMR (400 MHz, CHLOROFORM-d) δ 8.61 (d, J = 2 Hz, 1H), 8.54 (dd, J = 4.8, 1.6 Hz, 1H), 7.75 (td, J = 1.6, 7.8 Hz, 1H), 7.29-7.26 (m, 1H), 4.78 - 4.68 (m, 1H), 3.01 - 2.90 (m, 1H), 2.72-2.62 (m, 3H), 1.59 -1.43 (m, 2H), 1.03 - 0.83 (t, 3H).

### Step 3: N-[(2S)-2-Hydroxy-2-(3-Pyridyl)Ethyl]-N-Propyl-2-[6-(Trifluoromethyl)-3-Pyridyl]Acetamide

To a solution of 2-[6-(trifluoromethyl)-3-pyridyl]acetic acid.HCl (134 mg, 555 umol) in DMF (2 mL) were added DIEA (215 mg, 1.66 mmol), (1S)-2-(propylamino)-1-(3-pyridyl)ethanol (100 mg, 555 umol) and T3P (424 mg, 666 umol). The resultant mixture was stirred at 20° C. for 2 h and concentrated. The crude product was purified by prep-HPLC (Waters Xbridge Prep OBD C18 150*40 mm*10 um column; 15-45% acetonitrile in an a 10 mM ammonium bicarbonate solution in water and in an a 0.05% ammonia solution in water, 8 min gradient) to obtain N-[(2S)-2-hydroxy-2-(3-pyridyl)ethyl]-N-propyl-2-[6-(trifluoromethyl)-3-pyridyl]acetamide (110 mg, 295 umol, 53%) as a white gum.

¹H NMR (400 MHz, CHLOROFORM-d) δ 8.66 - 8.57 (m, 2H), 8.55 - 8.49 (m, 1H), 7.84 (dd, J = 1.4, 7.8 Hz, 1H), 7.81 - 7.71 (m, 1H), 7.71 - 7.61 (m, 1H), 7.38 - 7.27 (m, 1H), 5.09 - 4.95 (m, 1H), 4.08 - 3.48 (m, 5H), 3.40 - 3.09 (m, 2H), 1.66 - 1.51 (m, 2H), 1.04 - 0.86 (m, 3H). LCMS (ESI for C18H20F3N3O2) [M+H]+: 368.1; (Rt: 2.707 min).

Single crystal X-ray confirmed the S-configuration of the product. This product was also compared with the method using protocol described for Compound 14.

The following compounds were synthesized according to the protocol described above.

| Name | Structure | NMR, MS | Compd # |
|---|---|---|---|
| 2-(3,4-dichlorophenyl) -N-[(2S)-2-hydroxy-2-(3-pyridyl)ethyl]-N-isopropyl-acetamide | 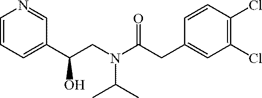 | ¹H NMR (400 MHz, Chloroform-d) δ 8.60 (d, J = 1.4 Hz, 1H), 8.55 (dd, J = 1.4, 4.6 Hz, 1H), 7.84 - 7.77 (m, 1H), 7.44 (d, J = 8.2 Hz, 1H), 7.38 (d, J = 2.0 Hz, 1H), 7.34 - 7.28 (m, 1H), 7.13 (dd, J = 2.0, 8.2 Hz, 1H), 5.64 - 5.56 (m, 1H), 4.94 - 4.87 (m, 1H), 4.18 - 4.06 (m, 1H), 3.87 - 3.80 (m, 1H), 3.80 - 3.73 (m, 2H), 3.23 - 3.14 (m, 1H), 1.27 (d, J = 6.6 Hz, 3H), 1.05 (d, J = 6.6 Hz, 3H) LCMS (ESI) m/z: 367.1 [M+H]⁺. | 18 |
| 2-(3,4-dichlorophenyl) -N-ethyl-N-[(2S)-2-hydroxy-2-(3-pyridyl)ethyl]ac etamide | 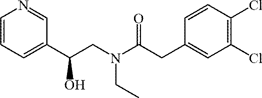 | ¹H NMR (400 MHz, Chloroform-d) δ 8.65 - 8.57 (m, 1H), 8.57 - 8.51 (m, 1H), 7.74 (br. d, J = 7.8 Hz, 1H), 7.43 (d, J = 8.2 Hz, 1H), 7.38 (d, J = 1.7 Hz, 1H), 7.32 - 7.28 (m, 1H), 7.15 - 6.99 (m, 1H), 5.09 - 4.99 (m, 1H), 4.68 (br. d, J = 3.4 Hz, 1H), 3.77 - 3.67 (m, 3H), 3.54 (dd, J = 2.5, 14.4 Hz, 1H), 3.41 - 3.29 (m, 1H), 3.20 (qd, J = 7.3, 14.8 Hz, 1H), 1.16 (t, J = 7.1 Hz, 3H); LCMS (ESI) m/z: 353.0 [M+H]⁺. | 20 |

### Example 11: Preparation of 2-[3-Chloro-4-(Trifluoromethoxy)Phenyl]-N-[(2S)-2-Hydroxy-2-(3-Pyridyl)Ethyl]-N-Propyl-Acetamide (Compound 21)

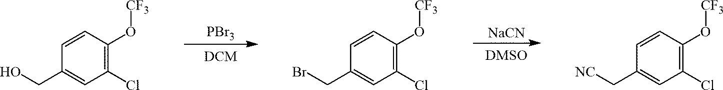

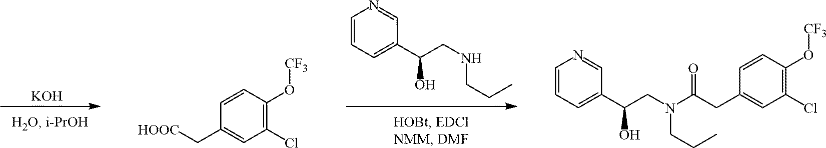

### Step 1: Preparation of 4-(bromomethyl)-2-chloro-1-(trifluoromethoxy)benzene.

To a solution of [3-chloro-4-(trifluoromethoxy)phenyl]methanol (1.00 g, 4.41 mmol) in dichloromethane (10 mL) was added PBr₃ (1.31 g, 4.85 mmol), then the mixture was stirred at 20° C. for 1 h and then at 50° C. for 2 h. The mixture was cooled to 0° C. and quenched by the slow addition of water (20 mL). The aqueous phase was extracted with dichloromethane (20 mL × 2). The combined organic layers were washed with brine (20 mL), dried over sodium sulfate and concentrated. The crude product was purified by flash column (ISCO 10 g silica, 0-30% ethyl acetate in petroleum ether, gradient over 30 min) to yield 4-(bromomethyl)-2-chloro-1-(trifluoromethoxy)benzene (725 mg, 2.50 mmol, 57%) as a yellow oil. ¹H NMR (400 MHz, Chloroform-d) δ 7.53 (d, J = 1.8 Hz, 1H), 7.36 - 7.28 (m, 2H), 4.43 (s, 2H).

### Step 2: Preparation of 2-[3-Chloro-4-(Trifluoromethoxy)Phenyl]Acetonitrile

To a solution of 4-(bromomethyl)-2-chloro-1-(trifluoromethoxy)benzene (725 mg, 2.50 mmol) in dimethylsulfoxide (8 mL) was added sodium cyanide (245 mg, 5.01 mmol). The mixture was stirred at 25° C. for 2 h. Water (20 mL) was added to the reaction, the reaction mixture was extracted with ethyl acetate (20 mL × 2). The combined organic layers were washed with brine (20 mL), dried over sodium sulfate, filtered, and concentrated. The crude product 2-[3-chloro-4-(trifluoromethoxy) phenyl]acetonitrile (520 mg, 2.21 mmol, 88%) was obtained as a yellow thick oil and used without further purification in the next step.

### Step 3: Preparation of 2-[3-Chloro-4-(Trifluoromethoxy)Phenyl]acetic Acid

To a solution of 2-[3-chloro-4-(trifluoromethoxy)phenyl]acetonitrile (300 mg, 1.27 mmol) in isopropanol (2 mL) was added potassium hydroxide (357 mg, 6.37 mmol) and water (1 mL). The mixture was stirred at 80° C. for 12 h. The reaction mixture was cooled to 25° C. and extracted with ethyl acetate (10 mL × 2). The aqueous phase was acidified with 1M HCl (10 mL) to pH=1~2 at 0° C. and then extracted with ethyl acetate (10 mL × 2). The combined organic layer was concentrated to dryness to give 2-[3-chloro-4-(trifluoromethoxy)phenyl]acetic acid (95 mg, 373 µmol, 29%) as a viscous yellow oil. This material was used directly in the next step without additional purification.

### Step 4: Preparation of 2-[3-Chloro-4-(Trifluoromethoxy)Phenyl]-N-[(2S)-2-Hydroxy-2-(3-Pyridyl)ethyl]-N-Propyl-Acetamide

To a solution of (1S)-2-(propylamino)-1-(3-pyridyl)ethanol (45 mg, 250 µmol) in dimethylformamide (0.5 mL) was added 2-[3-chloro-4-(trifluoromethoxy)phenyl]acetic acid (70 mg, 275 µmol), 1-hydroxybenzotriazole (41 mg, 300 µmol), N-(3-dimethylaminopropyl)-N′-ethylcarbodiimide (58 mg, 300 µmol) and N-methylmorpholine (76 mg, 750 µmol, 82 µL). Then the mixture stirred at 20° C. for 2 h. The reaction mixture was purified directly by prep-HPLC (Waters Xbridge BEH C18 100 × 30 mm × 10 um column; 33-55% acetonitrile in a 10 mM ammonium bicarbonate solution in water, 10 min gradient) to afford 2-[3-chloro-4-(trifluoromethoxy)phenyl]-N-[(2S)-2-hydroxy-2-(3-pyridyl)ethyl]-N-propyl-acetamide (5 mg, 13 µmol, 5%) as a pale yellow thick oil. ¹H NMR (400 MHz, Chloroform-d) δ 8.58 (s, 1H), 8.53 (d, J = 4.5 Hz, 1H), 7.74 (br. d, J = 7.9 Hz, 1H), 7.39 (s, 1H), 7.34 - 7.28 (m, 1H), 7.26 (br. s, 1H), 7.20 (dd, J = 1.5, 8.4 Hz, 1H), 5.04 (br. d, J = 6.4 Hz, 1H), 4.73 - 4.65 (s, 1H), 3.79 - 3.69 (m, 3H), 3.53 (dd, J = 2.4, 14.4 Hz, 1H), 3.31 - 3.20 (m, 1H), 3.17 - 3.08 (m, 1H), 1.62 - 1.53 (m, 2H), 0.95 - 0.87 (m, 3H) LCMS (ESI) m/z: 417.0 [M+H]⁺.

### Example 12: Preparation of N-(5,6-Dichloroindan-2-yl)-N-[(2S)-2-Hydroxy-2-(3-Pyridyl)Ethyl]Propenamide (Compound 22) and N-(5,6-Dichloroindan-2-yl)-N-[(2R)-2-Hydroxy-2-(3-Pyridyl)Ethyl]Propenamide (Compound 23)

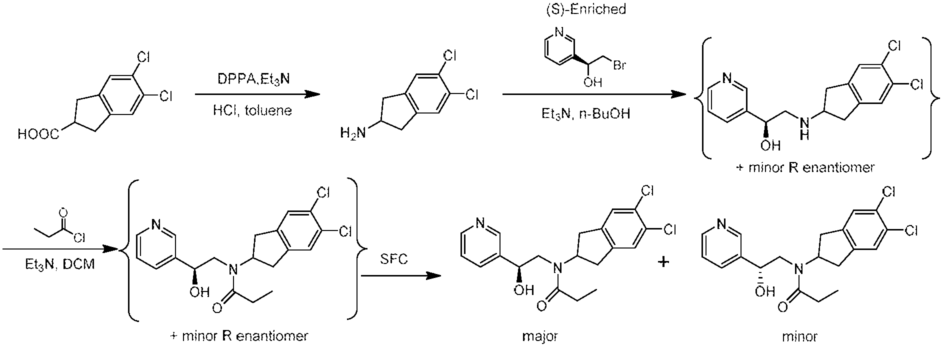 [SMILES: O=C(Cc1ccc(Cl)c(Cl)c1)N(CC1COC1)C[C@@H](O)c1cccnc1]

### Step 1: Preparation of 5,6-Dichloroindan-2-Amine

To a solution of 5,6-dichloroindane-2-carboxylic acid (1 g, 4.33 mmol) in toluene (20 mL) were added diphenylphosphoryl azide (1.19 g, 4.33 mmol) and triethylamine (657 mg, 6.49 mmol). The mixture was stirred at 20° C. for 1 h and then at 90° C. for 2 h and cooled to 20° C. The reaction mixture was then treated with HCl (6 M, 2.16 mL) and was stirred at 20° C. for 16 h. The reaction mixture was then filtered and the solids were dried in vacuo. The crude material was purified by prep-HPLC (Waters Xbridge Prep OBD C18 150 × 40 10 um column; 20-50% acetonitrile in an a 10 mM ammonium bicarbonate solution in water, 8 min gradient) to give 5,6-dichloroindan-2-amine (530 mg, 2.62 mmol, 61% as a pale yellow oil. ¹H NMR (400 MHz, Dimethylsulfoxide-d₆) δ 7.43 (s, 2H), 3.79 - 3.63 (m, 1H), 3.00 (dd, J = 6.6, 16.0 Hz, 2H), 2.55 (dd, J = 5.2, 16.1 Hz, 2H), 1.68 (br. s, 2H).

### Step 2: Preparation of (1S)-2-[(5,6-Dichloroindan-2-yl)Amino]-1-(3-Pyridyl)Ethanol and (1R)-2-[(5,6-Dichloroindan-2-yl)Amino]-1-(3-Pyridyl)Ethanol

To a solution of (1S)-2-bromo-1-(3-pyridyl)ethanol (624 mg, 3.09 mmol) in n-butanol (8 mL) were added 5,6-dichloroindan-2-amine (0.52 g, 2.57 mmol) and triethylamine (312 mg, 3.09 mmol). The resultant mixture was stirred at 120° C. for 16 h and concentrated. The crude product was purified by prep-HPLC (Welch Xtimate C18 250 × 50 10 um column; 20-60% acetonitrile in an a 10 mM ammonium bicarbonate solution in water, 10 min gradient) to give (1S)-2-[(5,6-dichloroindan-2-yl)amino]-1-(3-pyridyl)ethanol (220 mg, 681 µmol, 26%) as a pale brown oil. LCMS (ESI) m/z: 323.0 [M+H]⁺.

### Step 3: Preparation of N-(5,6-Dichloroindan-2-yl)-N-[(2S)-2-Hydroxy-2-(3-Pyridyl)Ethyl]Propanamide and N-(5,6-Dichloroindan-2-yl)-N-[(2R)-2-Hydroxy-2-(3-Pyridyl)Ethyl]Propanamide

To a solution of (1S)-2-[(5,6-dichloroindan-2-yl)amino]-1-(3-pyridyl)ethanol (150 mg, 464.09 µmol) in dichloromethane (2 mL) were added triethylamine (94 mg, 928 µmol) and propionyl chloride (43 mg, 464 µmol) at 0° C. The mixture was stirred at 0° C. for 1 h and concentrated to dryness. The crude product was purified by prep-HPLC (Waters Xbridge Prep OBD C18 150 × 40 10 um column; 35-60% acetonitrile in an a 10 mM ammonium bicarbonate solution in water, 8 min gradient) and then by preparative SFC (DAICEL CHIRALCEL OJ (250 mm × 30 mm,10 um) column, 40° C., eluting with 40% methanol containing 0.1%ammonium hydroxide in a flow of 70 g/min CO2 at 100 bar) to give N-(5,6-dichloroindan-2-yl)-N-[(2S)-2-hydroxy-2-(3-pyridyl)ethyl]propanamide (18 mg, 48 µmol, 10%) as a white solid and N-(5,6-dichloroindan-2-yl)-N-[(2R)-2-hydroxy-2-(3-pyridyl)ethyl]propanamide (3 mg, 6.98 µmol, 2%) as a white solid. ¹H NMR (400 MHz, Chloroform-d) for (S)-enantiomer: δ 8.67 - 8.38 (m, 2H), 7.72 -7.57 (m, 1H), 7.37 - 7.27 (m, 3H), 5.36 (br. s, 1H), 4.99 - 4.70 (m, 2H), 3.76 (br. dd, J = 9.0, 14.5 Hz, 1H), 3.30 - 3.17 (m, 2H), 3.03 (td, J = 8.3, 16.1 Hz, 2H), 2.82 (br. dd, J = 6.8, 16.6 Hz, 1H), 2.66 - 2.45 (m, 2H), 1.24 (br. t, J = 7.2 Hz, 3H); LCMS (ESI) m/z: 379.0 [M+H]⁺. ¹H NMR (400 MHz, Chloroform-d) for (R)-enantiomer: δ 8.60 - 8.42 (m, 2H), 7.63 (br. d, J = 7.8 Hz, 1H), 7.37 - 7.27 (m, 3H), 5.36 (br. s, 1H), 4.97 -4.77 (m, 2H), 3.76 (br. dd, J = 9.0, 14.4 Hz, 1H), 3.28 - 3.17 (m, 2H), 3.03 (td, J = 8.1, 16.1 Hz, 2H), 2.82 (br. dd, J = 7.2, 16.6 Hz, 1H), 2.65 - 2.38 (m, 2H), 1.24 (br. t, J = 7.3 Hz, 3H); LCMS (ESI) m/z: 379.0 [M+H]⁺. (Note: Though the reactions were carried with a chiral alcohol, the material still contained some percentage of the other isomer, which was separated through chiral HPLC).

### Example 13: Preparation of 2-(4-Chloro-3-Cyano-Phenyl)-N-[(2S)-2-Hydroxy-2-(3-Pyridyl)Ethyl]-N-Propyl-Acetamide (Compound 24)

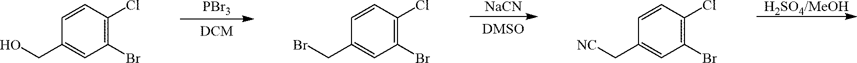 [SMILES: O=C(Cc1ccc(Cl)c(Cl)c1)N(C[C@@H](O)c1cccnc1)C1COC1]

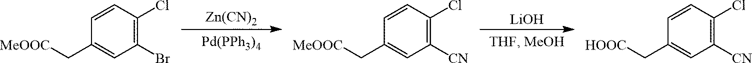 [SMILES: CC1(CN(C[C@@H](O)c2cccnc2)C(=O)Cc2ccc(Cl)c(Cl)c2)CC1]

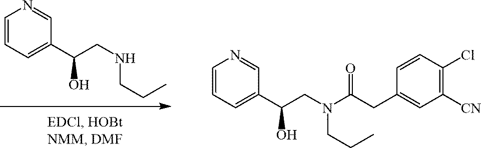 [SMILES: CCC(=O)N(CCc1ccc(C(F)(F)F)nc1)C[C@@H](O)c1cccnc1]

### Step 1: Preparation of 2-bromo-4-(bromomethyl)-1-chloro-benzene.

To a solution of (3-bromo-4-chloro-phenyl)methanol (3 g, 13.55 mmol) in dichloromethane (50 mL) was added PBr₃ (4.03 g, 14.90 mmol). The mixture was stirred at 20° C. for 1 h and was quenched by addition of methanol (10 mL) at 0° C. Water (20 mL) was added and the aqueous phase was extracted with dichloromethane (50 mL × 2). The combined organic layers were washed with brine (20 mL × 2), dried over sodium sulfate, filtered and concentrated under reduced pressure to give crude 2-bromo-4-(bromomethyl)-1-chloro-benzene (3.82 g, 12.09 mmol, 89%) as a pale yellow oil. ¹H NMR (400 MHz, Chloroform-d) δ 7.68 - 7.65 (m, 1H), 7.45 - 7.41 (m, 1H), 7.27 (m, 1H), 4.41 (s, 2H).

### Step 2: Preparation of 2-(3-bromo-4-chloro-phenyl)acetonitrile.

To a solution of 2-bromo-4-(bromomethyl)-1-chloro-benzene (3.7 g, 13.01 mmol) in dimethylsulfoxide (30 mL) was added sodium cyanide (1.28 g, 26.02 mmol). The mixture was stirred at 20° C. for 2 h and was quenched by the addition of water (20 mL) and the dimethylsulfoxide phase was collected. The water phase was then extracted with ethyl acetate (40 mL × 2). The combined organic layers were washed with brine (10 mL × 2), dried over sodium sulfate, filtered and concentrated under reduced pressure. The crude product was purified by flash column (ISCO 40 g silica, 0-15% ethyl acetate in petroleum ether, gradient over 20 min) to give 2-(3-bromo-4-chloro-phenyl)acetonitrile (2.3 g, 7.98 mmol, 61%) as a yellow oil. ¹H NMR (400 MHz, Chloroform-d) δ 7.68 - 7.61 (m, 1H), 7.52 - 7.44 (m, 1H), 7.27 (dd, J = 6.6, 7.8 Hz, 1H), 3.75 (s, 2H).

### Step 3: Preparation of Methyl 2-(3-Bromo-4-Chloro-Phenyl)Acetate

A solution of 2-(3-bromo-4-chloro-phenyl)acetonitrile (2 g, 8.68 mmol) in a mixture of sulfuric acid (2.55 g, 26.03 mmol, 1.39 mL) and water (235 mg, 13.02 mmol,) was stirred at 65° C. for 0.5 h. After cooling to 50° C., methanol (20 mL) was slowly added. Then the mixture was stirred at 90° C. for 12 h followed by the addition of water (10 mL) and the aqueous phase was then extracted with ethyl acetate (50 mL × 2). The combined organic layers were dried over sodium sulfate, filtered and concentrated under reduced pressure to yield crude methyl 2-(3-bromo-4-chloro-phenyl)acetate (2 g, 7.59 mmol, 88%) as a pale yellow oil. The material was used in the next step without further purification. LCMS (ESI) m/z: 264.9 [M+H]⁺.

### Step 4: Preparation of Methyl 2-(4-Chloro-3-Cyano-Phenyl)Acetate

To a solution of methyl 2-(3-bromo-4-chloro-phenyl)acetate (2 g, 7.59 mmol) in dimethylformamide (20 mL) was added zinc cyanide (980 mg, 8.35 mmol) and palladium-tetrakis(triphenylphosphine) (877 mg, 759 µmol). The mixture was stirred at 100° C. for 12 h and then treated with water (20 mL). The aqueous phase was then extracted with ethyl acetate (50 mL). The combined organic layers were washed with brine (20 mL × 2), dried over sodium sulfate, filtered and concentrated under reduced pressure. The crude residue was purified by flash column (ISCO 20 g silica, 20-25% ethyl acetate in petroleum ether, gradient over 20 min) to yield methyl 2-(4-chloro-3-cyanophenyl)acetate (0.6 g, 2.86 mmol, 38%) as a white solid.

### Step 5: Preparation of 2-(4-Chloro-3-Cyano-Phenyl)Acetic Acid

To a solution of methyl 2-(4-chloro-3-cyano-phenyl)acetate (300 mg, 1.43 mmol) in tetrahydrofuran (2 mL) and methanol (2 mL) was added lithium hydroxide hydrate (2 M, 2.15 mL). The mixture was stirred at 20° C. for 2 h and then concentrated in vacuum. Water (2 mL) was added and the aqueous phase was extracted with ethyl acetate (5 mL × 3). The combined organic layers were dried over sodium sulfate, filtered and concentrated under reduced pressure. The crude 2-(4-chloro-3-cyanophenyl)acetic acid (200 mg) was used in the next step without further purification.

### Step 6: Preparation of 2-(4-Chloro-3-Cyano-Phenyl)-N-[(2S)-2-Hydroxy-2-(3-Pyridyl)ethyl]-N-Propyl-Acetamide

To a solution of 2-(4-chloro-3-cyano-phenyl)acetic acid (99 mg, 505 µmol) in dimethylformamide (3 mL) was added N-(3-dimethylaminopropyl)-N′-ethylcarbodiimide (89 mg, 466 µmol), 1-hydroxybenzotriazole (63 mg, 466 µmol), N-methylmorpholine (118 mg, 1.17 mmol, 128 µL) and (1S)-2-(propylamino)-1-(3-pyridyl)ethanol (70 mg, 388 µmol). The mixture was stirred at 15° C. for 3 h and then concentrated in vacuum. The residue was purified by prep-HPLC (Welch Xtimate C18 150 × 25 mm × 5 um column; 25-55% acetonitrile in a 10 mM ammonium bicarbonate solution in water, 10 min gradient) to give 2-(4-chloro-3-cyano-phenyl)-N-[(2S)-2-hydroxy-2-(3-pyridyl)ethyl]-N-propyl-acetamide (34 mg, 92 µmol, 24%) as a pale yellow thick oil. ¹H NMR (400 MHz, Chloroform-d) δ 8.69 - 8.57 (m, 1H), 8.57 - 8.52 (m, 1H), 7.79 - 7.71 (m, 1H), 7.59 - 7.54 (m, 1H), 7.53 - 7.48 (m, 1H), 7.48 - 7.42 (m, 1H), 7.33 - 7.28 (m, 1H), 5.09 - 4.97 (m, 1H), 4.47 - 4.34 (m, 1H), 3.79 - 3.69 (m, 3H), 3.53 (dd, J = 2.6, 14.3 Hz, 1H), 3.39 -3.12 (m, 2H), 1.69 - 1.61 (m, 2H), 1.02 - 0.86 (m, 3H); LCMS (ESI) m/z: 358.1 [M+H]⁺.

The following compounds was synthesized according to the protocol described for the compound 24:

| | | | |
|---|---|---|---|
| Name | Structure | NMR, MS | Compd# |
| 2-(3,4-dichlorophenyl) -N-[(2S)-2-hydroxy-2-(3-pyridyl)ethyl]-N-(oxetan-3-ylmethyl)acetamide | 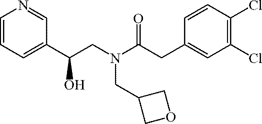 | ¹H NMR (400 MHz, Chloroform-d) δ 8.60 - 8.48 (m, 2H), 7.72 (br. d, J = 7.7 Hz, 1H), 7.43 (d, J = 8.3 Hz, 0.6H), 7.39 - 7.33 (m, 1.4H), 7.31 - 7.27 (m, 0.5H), 7.25 (s, 0.5H), 7.10 (dd, J = 1.6, 8.2 Hz, 0.6H), 7.01 (br. d, J = 8.3 Hz, 0.4H), 5.00 (br. s, 0.6H), 4.90 (br. d, J = 6.8 Hz, 0.4H), 4.83 - 4.69 (m, 2H), 4.48 (td, J = 6.4, 13.0 Hz, 0.7H), 4.34 (q, J = 6.4 Hz, 0.3H), 4.24 (br. s, 0.5H), 3.95 (br. dd, J = 7.0, 13.9 Hz, 0.6H), 3.81 - 3.47 (m, 5.5H), 3.37 - 3.12 (m, 1.4H); LCMS (ESI) m/z: 395.1 [M+H]⁺. | 25 |
| 2-(3,4-dichlorophenyl) -N-[(2S)-2-hydroxy-2-(3-pyridyl)ethyl]-N-(oxetan-3-yl)acetamide | 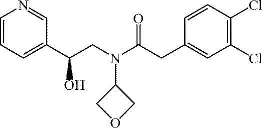 | ¹H NMR (400 MHz, Chloroform-d) δ 8.70 - 8.53 (m, 2H), 7.83 - 7.64 (m, 1H), 7.46 - 7.28 (m, 3H), 7.07 (br. d, J = 8.4 Hz, 1H), 5.17 - 5.02 (m, 1H), 4.86 - 4.58 (m, 5H), 3.88 (br. d, J = 5.3 Hz, 2H), 3.81 - 3.43 (m, 3H).¹H NMR (400 MHz, Methanol-d₄) δ 8.67 - 8.55 (m, 1H), 8.53 - 8.42 (m, 1H), 7.99 - 7.86 (m, 1H), 7.51 - 7.33 (m, 3H), 7.24 - 7.05 (m, 1H), 5.35 - 5.04 (m, 1H), 4.96 - 4.68 (m, 4H), 4.67 - 4.56 (m, 1H), 3.91 - 3.60 (m, 4H); LCMS (ESI) m/z: 380.9 [M+H]⁺. | 26 |
| 2-(3,4-dichlorophenyl) -N-[(2S)-2-hydroxy-2-(3-pyridyl)ethyl]-N-[(1-methylcyclopropyl) methyl]acetamide | 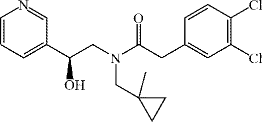 [SMILES: NCCCCCN(CC(O)c1cccnc1)C(=O)Cc1ccc(Cl)c(Cl)c1] | ¹H NMR (400 MHz, Chloroform-d) δ 8.65 - 8.57 (m, 1H), 8.54 (dd, J = 1.6, 4.8 Hz, 1H), 7.76 (br. d, J = 7.9 Hz, 1H), 7.43 (d, J = 8.2 Hz, 1H), 7.37 (d, J = 2.0 Hz, 1H), 7.32 - 7.28 (m, 1H), 7.12 (dd, J = 2.0, 8.2 Hz, 1H), 5.09 (br. d, J = 8.7 Hz, 1H), 4.71 (d, J = 3.9 Hz, 1H), 4.00 (dd, J = 8.9, 14.5 Hz, 1H), 3.90 - 3.71 (m, 2H), | 27 |
| | | 3.56 - 3.44 (m, 2H), 2.91 (d, J = 14.8 Hz, 1H), 1.07 (s, 3H), 0.59 - 0.31 (m, 4H); LCMS (ESI) m/z: 393.1 [M+H]⁺. | |

### Example 14: Preparation of Methyl N-[2-Hydroxy-2-(3-Pyridyl)Ethyl]-N-[2-[6-(Trifluoromethyl)-3-Pyridyl]ethyl]Carbamate (Compound 28)

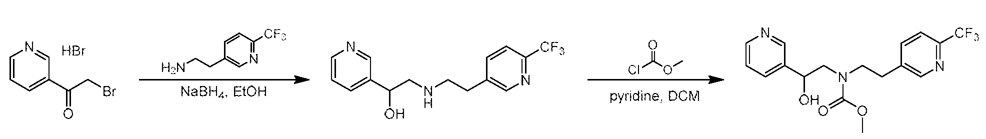

### Step 1: Preparation of 1-(3-Pyridyl)-2-[2-[6-(Trifluoromethyl)-3-Pyridyl]ethylamino]Ethanol

To a solution of 2-bromo-1-(pyridin-3-yl)ethan-1-one (600 mg, 2.14 mmol HBr) in ethanol (6 mL) was added sodium borohydride (300 mg, 7.92 mmol). The mixture was stirred at 25° C. for 2 h. The reaction mixture was filtered and 2-[6-(trifluoromethyl)-3-pyridyl]ethanamine (812 mg, 4.27 mmol) was added to the filtrate and the reaction mixture was stirred at 80° C. for 6 h and concentrated. The crude product was purified by prep-HPLC (Kromasil C18 250 × 50 mm × 10 um column; 5-45% acetonitrile in an a 0.04% ammonia and 10 mM ammonium bicarbonate solution in water, 10 min gradient) to give 1-(3-pyridyl)-2-[2-[6-(trifluoromethyl)-3-pyridyl]ethylamino]ethanol (276 mg, 887 µmol, 42%) as a yellow thick oil; LCMS (ESI) m/z: 312.1 [M+H]⁺.

### Step 2: Preparation of Methyl N-[2-Hydroxy-2-(3-Pyridyl)Ethyl]-N-[2-[6-(Trifluoromethyl)-3-Pyridyl]ethyl]Carbamate

To a solution of 1-(3-pyridyl)-2-[2-[6-(trifluoromethyl)-3-pyridyl]ethylamino]ethanol (150 mg, 482 µmol) in dichloromethane (2 mL) was added pyridine (114 mg, 1.45 mmol) and methyl chloroformate (46 mg, 482 µmol). The mixture was stirred at -10° C. for 30 min and then concentrated. The crude product thus obtained was purified by prep-HPLC (Waters Xbridge Prep OBD C18 150 × 40 mm × 10 um column; 15-45% acetonitrile in an a 10 mM ammonium bicarbonate solution in water, 8 min gradient) to give methyl N-[2-hydroxy-2-(3-pyridyl)ethyl]-N-[2-[6-(trifluoromethyl)-3-pyridyl]ethyl]carbamate (84 mg, 224 µmol, 46%) as a pale yellow thick oil. ¹H NMR (400 MHz, Chloroform-d) δ 8.65 - 8.50 (m, 3H), 7.83 - 7.59 (m, 3H), 7.31 (dd, J = 4.9, 7.7 Hz, 1H), 5.03 (br. s, 1H), 3.90 (br. s, 1H), 3.67 (s, 3H), 3.62 - 3.21 (m, 4H), 2.89 (br. s, 2H). ¹H NMR (400 MHz, Methanol-d₄) δ 8.64 - 8.40 (m, 3H), 7.96 - 7.82 (m, 2H), 7.74 (d, J = 8.1 Hz, 1H), 7.44 (dd, J = 4.9, 7.7 Hz, 1H), 5.05 - 4.90 (m, 1H), 3.67 - 3.56 (m, 2H), 3.56 - 3.47 (m, 4H), 3.47 - 3.34 (m, 1H), 3.01 (br. t, J = 6.7 Hz, 2H); LCMS (ESI) m/z: 370.0 [M+H]⁺.

### Example 15: Preparation of 3-(3,4-Dichlorophenyl)-1-[(2S)-2-Hydroxy-2-(3-Pyridyl)ethyl]-1-Isopropyl-Urea (Compound 29)

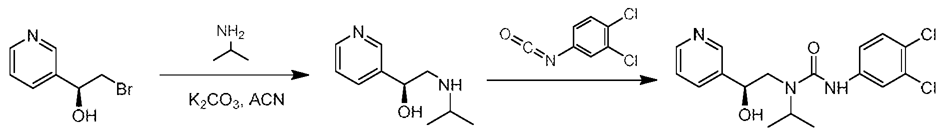

### Step 1: Preparation of (1S)-2-(Isopropylamino)-1-(3-Pyridyl)Ethanol

To a solution of (1S)-2-bromo-1-(3-pyridyl)ethanol (2 g, 9.90 mmol) in acetonitrile (10 mL) was added potassium carbonate (5.47 g, 39.59 mmol). The mixture was stirred at 80° C. for 2 h and filtered followed by the addition of propan-2-amine (1.40 g, 23.76 mmol). The reaction solution was stirred at 80° C. for 24 h and concentrated under reduced pressure. The crude product thus obtained was purified by prep-HPLC (Kromasil C18 250 × 50 mm × 10 um column; 1-25% acetonitrile in an a 0.04% ammonia and 10 mM ammonium bicarbonate solution in water, 10 min gradient) to give (1S)-2-(isopropylamino)-1-(3-pyridyl)ethanol (640 mg, 3.55 mmol, 36%) as a pale yellow thick oil. ¹H NMR (400 MHz, Dimethylsulfoxide-d₆) δ 8.54 (d, J = 1.8 Hz, 1H), 8.44 (dd, J = 1.5, 4.7 Hz, 1H), 7.80 - 7.68 (m, 1H), 7.33 (dd, J = 4.9, 7.7 Hz, 1H), 5.43 (br. s, 1H), 4.63 (dd, J = 4.6, 7.8 Hz, 1H), 2.76 - 2.59 (m, 3H), 0.95 (dd, J = 6.4, 7.6 Hz, 6H); LCMS (ESI) m/z: 180.9 [M+H]⁺.

### Step 2: Preparation of 3-(3,4-Dichlorophenyl)-1-[(2S)-2-Hydroxy-2-(3-Pyridyl)Ethyl]-1-Isopropyl-Urea

To a solution of (1S)-2-(isopropylamino)-1-(3-pyridyl)ethanol (150 mg, 832 µmol) in dichloromethane (2 mL) was added 1,2-dichloro-4-isocyanato-benzene (156 mg, 832 µmol). The mixture was stirred at 40° C. for 1 h and concentrated. The crude product was purified by prep-HPLC (Waters Xbridge BEH C18 100 × 30 mm × 10 um column; 40-65% acetonitrile in an a 10 mM ammonium bicarbonate solution in water, 10 min gradient) to give 3-(3,4-dichlorophenyl)-1-[(2S)-2-hydroxy-2-(3-pyridyl)ethyl]-1-isopropyl-urea (114 mg, 308 µmol, 37%) as a pale yellow solid. ¹H NMR (400 MHz, Chloroform-d) δ 9.03 (br. s, 1H), 8.59 - 8.46 (m, 2H), 7.79 (td, J = 1.7, 7.9 Hz, 1H), 7.52 (d, J = 2.6 Hz, 1H), 7.36 (dd, J = 4.9, 7.8 Hz, 1H), 7.32 - 7.27 (m, 1H), 7.20 (dd, J = 2.5, 8.7 Hz, 1H), 5.90 (br. s, 1H), 4.87 (br. d, J = 9.2 Hz, 1H), 4.39 (td, J = 6.7, 13.4 Hz, 1H), 3.53 (dd, J = 9.4, 15.9 Hz, 1H), 3.20 (br. d, J = 15.3 Hz, 1H), 1.29 - 1.18 (m, 3H), 1.09 (d, J = 6.6 Hz, 3H). ¹H NMR (400 MHz, Methanol-d4) δ 8.63 (d, J = 2.0 Hz, 1H), 8.49 (dd, J = 1.5, 5.1 Hz, 1H), 8.00 - 7.92 (m, 1H), 7.66 (d, J = 2.4 Hz, 1H), 7.47 (dd, J = 5.0, 7.8 Hz, 1H), 7.39 (d, J = 8.8 Hz, 1H), 7.21 (dd, J = 2.5, 8.7 Hz, 1H), 4.99 (dd, J = 2.2, 8.8 Hz, 1H), 4.34 (spt, J = 6.8 Hz, 1H), 3.58 (dd, J = 8.8, 15.7 Hz, 1H), 3.37 (dd, J = 2.5, 15.8 Hz, 1H), 1.24 (d, J = 6.8 Hz, 3H), 1.12 (d, J = 6.6 Hz, 3H); LCMS (ESI) m/z: 368.1 [M+H]⁺.

The following compounds were synthesized according to the protocol described above.

| Name | Structure | NMR, MS | Compd# |
|---|---|---|---|
| N-[(2S)-2-hydroxy-2-(3-pyridyl)ethyl]-N-[2-[6-(trifluoromethyl)-3-pyridyl]ethyl]prop enamide | 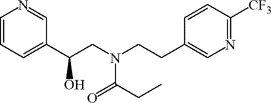 | ¹H NMR (400 MHz, Methanol-d₄) δ 8.60 (t, J = 6.7 Hz, 2H), 8.47 (ddd, J = 1.3, 4.9, 10.4 Hz, 1H), 7.97 - 7.88 (m, 2H), 7.75 (t, J = 7.8 Hz, 1H), 7.45 (dt, J = 5.0, 7.4 Hz, 1H), 5.08 - 4.92 (m, 1H), 3.87 - 3.56 (m, 3H), 3.47 - 3.39 (m, 1H), 3.09 - 2.96 (m, 2H), 2.51 - 2.14 (m, 2H), 1.01 (q, J = 7.4 Hz, 3H); LCMS (ESI) m/z: 368.1 [M+H]⁺. | 30 |
| 2-(4-chloro-2-fluoro-phenyl)-N-[2-hydroxy-2-(3- | 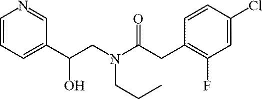 [SMILES: CCCN(CC(O)c1cncc(Br)c1)C(=O)Cc1ccc(C(F)(F)F)nc1] | ¹H NMR (400 MHz, Methanol-d₄) δ 8.64 -8.53 (m, 1H), 8.51 - 8.42 (m, 1H), 8.00 -7.83 (m, 1H), 7.50 - 7.36 (m, 1H), 7.27 -7.11 (m, 3H), 5.08 - 4.97 (m, 1H), 3.90 - | 31 |
| pyridyl)ethyl]-N-propyl-acetamide | | 3.64 (m, 3H), 3.59 - 3.22 (m, 3H), 1.71 -1.54 (m, 2H), 0.98 - 0.82 (m, 3H); LCMS (ESI) m/z: 351.1 [M+H]⁺. | |
| N-(2-hydroxy-2-pyridazin-3-yl-ethyl)-N-propyl-2-[6-(trifluoromethyl)-3-pyridyl]acetamide | 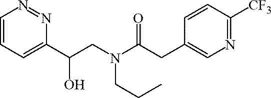 | ¹H NMR (400 MHz, Chloroform-d) δ 9.10 (dd, J = 1.5, 4.9 Hz, 1H), 8.56 (s, 1H), 7.79 (dd, J = 1.1, 8.6 Hz, 2H), 7.68 (d, J = 8.4 Hz, 1H), 7.49 (dd, J = 5.0, 8.5 Hz, 1H), 5.40 (br. s, 1H), 5.31 - 5.22 (m, 1H), 3.99 - 3.86 (m, 2H), 3.80 (s, 2H), 3.49 - 3.28 (m, 2H), 1.77 - 1.67 (m, 2H), 0.98 (t, J = 7.4 Hz, 3H); LCMS (ESI) m/z: 369.2 [M+H]⁺. | 32 |
| N-(5-aminopentyl)-2-(3,4-dichlorophenyl)-N-[2-hydroxy-2-(3-pyridyl)ethyl]acet amide | 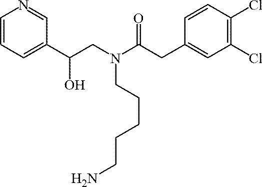 | ¹H NMR (400 MHz, Methanol-d₄) δ 8.63 -8.53 (m, 1H), 8.52 - 8.42 (m, 1H), 7.99 -7.81 (m, 1H), 7.54 - 7.33 (m, 3H), 7.14 (ddd, J = 1.8, 8.3, 14.7 Hz, 1H), 5.15 -4.94 (m, 1H), 3.91 - 3.65 (m, 3H), 3.64 -3.37 (m, 3H), 2.73 (td, J = 7.4, 12.2 Hz, 2H), 1.68 - 1.47 (m, 4H), 1.39 - 1.25 (m, 2H); LCMS (ESI) m/z: 410.0 [M+H]⁺. | 33 |

### Example 16: Preparation of N-[(5,6-Dichloroindan-2-yl)Methyl]-N-[(2S)-2-Hydroxy-2-(3-Pyridyl)ethyl]Propenamide (Compound 34)

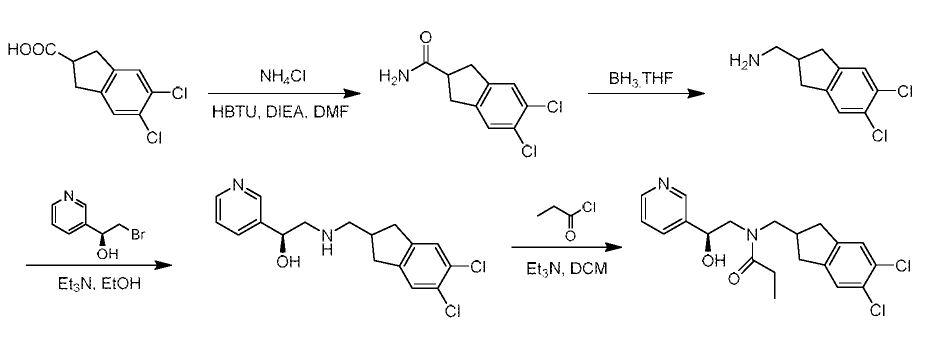

### Step 1: Preparation of 5,6-dichloroindane-2-carboxamide.

To a solution of 5,6-dichloroindane-2-carboxylic acid (1.5 g, 6.49 mmol) in dimethylformamide (20 mL) were added ammonium chloride (382 mg, 7.14 mmol), N,N,N′,N′-tetramethyl-O-(1H-benzotriazol-1-yl)uronium hexafluorophosphate (2.46 g, 6.49 mmol) and diisopropylethylamine (2.52 g, 19.47 mmol). The mixture was stirred at 25° C. for 1 h. Water (30 mL) was added and the aqueous layer was extracted with ethyl acetate (30 mL × 2). The combined organic layers were washed with brine (15 mL), dried over sodium sulfate, filtered and concentrated to dryness. The crude product was purified by flash column (ISCO 20 g silica, 0-10% methanol in dichloromethane, gradient over 30 min) to yield 5,6-dichloroindane-2-carboxamide (1.16 g, 5.04 mmol, 78%) as a white solid. ¹H NMR (400 MHz, Dimethylsulfoxide-d₆) δ 7.48 - 7.41 (m, 3H), 6.90 (br. s, 1H), 3.25 - 3.14 (m, 1H), 3.11 - 2.96 (m, 4H).

### Step 2: Preparation of (5,6-Dichloroindan-2-yl)Methanamine.

To a solution of 5,6-dichloroindane-2-carboxamide (1 g, 4.35 mmol) in tetrahydrofuran (15 mL) was added borane-tetrahydrofuran (1 M, 8.69 mL)). The mixture was stirred at 75° C. for 16 h and treated with 1.5 M HCl (20 mL) and methanol (20 mL) and stirred further at 75° C. for 2 h. Then the mixture was basified with 2N sodium hydroxide, extracted with ethyl acetate (50 mL × 2). The combined organic layers were washed with brine (30 mL), dried over sodium sulfate, filtered and concentrated to dryness. The crude product (5,6-dichloroindan-2-yl)methanamine (0.85 g) was obtained as a white solid and used in the next step without further purification. ¹H NMR (400 MHz, Dimethylsulfoxide-d₆) δ 7.46 (s, 2H), 6.51 (br. s, 2H), 3.07 - 2.93 (m, 2H), 2.83 - 2.74 (m, 2H), 2.73 - 2.64 (m, 3H).

### Step 3: Preparation of (1S)-2-[(5,6-Dichloroindan-2-yl)Methylamino]-1-(3-Pyridyl)Ethanol

To a solution of (1S)-2-bromo-1-(3-pyridyl)ethanol (729 mg, 3.61 mmol) in ethanol (8 mL) were added (5,6-dichloroindan-2-yl)methanamine (780 mg, 3.61 mmol) and triethylamine (438 mg, 4.33 mmol). The mixture was stirred at 80° C. for 16 h and concentrated. The resultant crude product was purified by prep-HPLC (Kromasil C18 250 × 50 5 um column; 20-60% acetonitrile in an a 10 mM ammonium bicarbonate solution in water, 10 min gradient) to give (1S)-2-[(5,6-dichloroindan-2-yl)methylamino]-1-(3-pyridyl)ethanol (180 mg, crude) as a pale yellow oil. ¹H NMR (400 MHz, Chloroform-d) δ 8.60 (d, J = 1.7 Hz, 1H), 8.57 - 8.51 (m, 1H), 7.77 - 7.68 (m, 1H), 7.32 - 7.29 (m, 1H), 7.28 - 7.27 (m, 2H), 4.73 (dd, J = 3.5, 9.2 Hz, 1H), 3.12 - 3.01 (m, 2H), 2.80 - 2.57 (m, 7H).

### Step 4: Preparation of N-[(5,6-Dichloroindan-2-yl)Methyl]-N-[(2S)-2-Hydroxy-2-(3-Pyridyl)ethyl]Propenamide

To a solution of (1S)-2-[(5,6-dichloroindan-2-yl)methylamino]-1-(3-pyridyl)ethanol (150 mg, 445 µmol) in dichloromethane (5 mL) were added triethylamine (90 mg, 890 µmol) and propionyl chloride (45 mg, 489 µmol) at 0° C. The mixture was stirred at 0° C. for 1 h and then concentrated to dryness. The crude product was purified by prep-HPLC (Waters Xbridge BEH C18 100 × 30 10 um column; 30-52% acetonitrile in an a 10 mM ammonium bicarbonate solution in water, 10 min gradient) to give N-[(5,6-dichloroindan-2-yl)methyl]-N-[(2S)-2-hydroxy-2-(3-pyridyl)ethyl]propanamide (68 mg, 173 µmol, 39%) as a pale yellow solid.

¹H NMR (400 MHz, Chloroform-d) δ 8.59 (d, J = 2.0 Hz, 1H), 8.54 (dd, J = 1.6, 4.8 Hz, 1H), 7.82 - 7.69 (m, 1H), 7.33 - 7.24 (m, 3H), 5.07 - 4.92 (m, 2H), 3.81 (dd, J = 8.1, 14.4 Hz, 1H), 3.50 (dd, J = 2.3, 14.4 Hz, 1H), 3.35 - 3.23 (m, 1H), 3.21 - 3.10 (m, 1H), 3.07 - 2.88 (m, 2H), 2.85 - 2.71 (m, 1H), 2.67 - 2.48 (m, 2H), 2.32 - 2.20 (m, 2H), 1.15 (t, J = 7.4 Hz, 3H); LCMS (ESI) m/z: 393.2 [M+H]⁺.

### Example 17: Preparation of Enantiomer 1 (Compound 35) and Enantiomer 2 (Compound 36) of 2-(3,4-Dichlorophenyl)-N-[2-Hydroxy-2-Pyridazin-3-yl-Ethyl]-N-Propyl-Acetamide

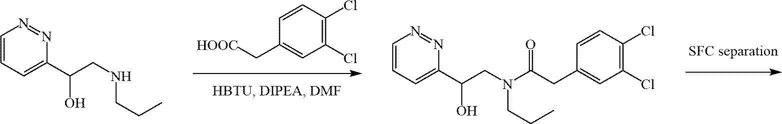

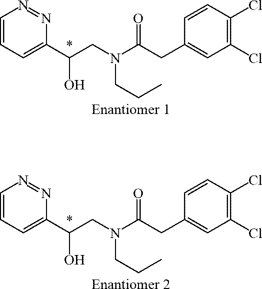

To a solution of 2-(propylamino)-1-pyridazin-3-yl-ethanol (330 mg, 1.82 mmol) in dimethylformamide (4 mL) were added 2-(3,4-dichlorophenyl)acetic acid (373 mg, 1.82 mmol), N,N,N′,N′-tetramethyl-O-(1H-benzotriazol-1-yl)uronium hexafluorophosphate (760 mg, 2.00 mmol) and diisopropylethylamine (706 mg, 5.46 mmol, 951 µL). The mixture was stirred at 20° C. for 1 h. The crude solution was purified by prep-HPLC (Kromasil C18 250 × 50 10 um column; 25-45% acetonitrile in an a 10 mM ammonium bicarbonate solution in water, 10 min gradient) to obtain 2-(3,4-dichlorophenyl)-N-[2-hydroxy-2-pyridazin-3-yl-ethyl]-N-propyl-acetamide which was chirally resolved by preparative SFC (Phenomenex-Cellulose-2 (250 mm × 30 mm, 10 um) column, 40° C., eluting with 40% ethanol containing 0.1%ammonium hydroxide in a flow of 70 g/min CO₂ at 100 bar) to yield enantiomer 1 (compound 35, 110 mg, 298 µmol, 16%) as a pale yellow solid and enantiomer 2 (compound 36, 115 mg, 310 µmol, 17%) as a pale yellow thick oil.

¹H NMR (400 MHz, Chloroform-d) for enantiomer 1: δ 9.09 (dd, J = 1.3, 4.9 Hz, 1H), 7.80 (dd, J = 1.1, 8.6 Hz, 1H), 7.48 (dd, J = 5.0, 8.5 Hz, 1H), 7.40 (d, J = 8.2 Hz, 1H), 7.31 (d, J = 1.8 Hz, 1H), 7.06 (dd, J = 2.1, 8.3 Hz, 1H), 5.62 (d, J = 4.9 Hz, 1H), 5.32 - 5.21 (m, 1H), 4.00 - 3.87 (m, 2H), 3.67 (s, 2H), 3.40 - 3.17 (m, 2H), 1.71 - 1.62 (m, 2H), 0.93 (t, J = 7.4 Hz, 3H); LCMS (ESI) m/z: 368.1 [M+H]⁺.

¹H NMR (400 MHz, Chloroform-d) for enantiomer 2: δ 9.09 (dd, J = 1.3, 4.9 Hz, 1H), 7.80 (d, J = 8.6 Hz, 1H), 7.48 (dd, J = 5.0, 8.5 Hz, 1H), 7.41 (d, J = 8.2 Hz, 1H), 7.31 (d, J = 1.8 Hz, 1H), 7.10 - 7.03 (m, 1H), 5.62 (d, J = 4.9 Hz, 1H), 5.29 - 5.22 (m, 1H), 3.99 - 3.85 (m, 2H), 3.67 (s, 2H), 3.40 - 3.14 (m, 2H), 1.70 -1.60 (m, 2H), 0.93 (t, J = 7.4 Hz, 3H); LCMS (ESI) m/z: 368.1 [M+H]⁺.

### Example 18: Preparation of N-[2-(5-Chloro-3-Pyridyl)-2-Hydroxy-Ethyl]-2-(3,4-Dichlorophenyl)-N-Propyl-Acetamide (Compound 37)

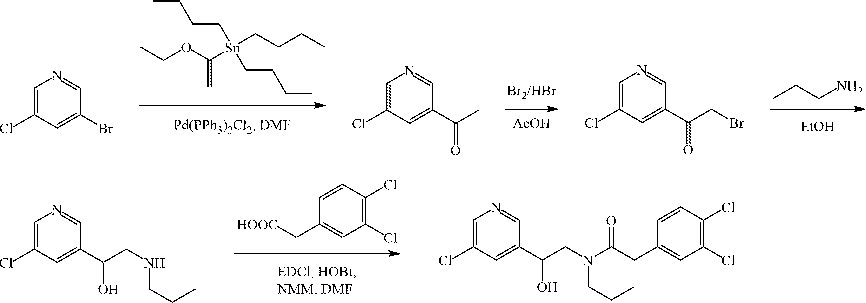

### Step 1: Preparation of 1-(5-Chloropyridin-3-yl)Ethan-1-One

To a solution of 3-bromo-5-chloro-pyridine (4 g, 20.79 mmol) in dimethylformamide (30 mL) were added bis(triphenylphosphine)palladium(II) dichloride(146 mg, 208 µmol) and tributyl(1-ethoxyvinyl)stannane (9.01 g, 24.94 mmol, 8.42 mL) under N₂. The mixture was stirred at 100° C. for 3 h and then the reaction mixture was treated with ethyl acetate (80 mL) and 8 g KF in water (40 mL) and stirred further for 1 h. The water phase was then extracted with ethyl acetate (100 mL × 2). The combined organic layers were washed with sodium bicarbonate (50 mL × 1), dried over sodium sulfate, filtered and concentrated under reduced pressure. The residue was treated with tetrahydrofuran (40 mL) and 2 M HCl (40 mL), and stirred at 25° C. for 12 h. Then tetrahydrofuran was concentrated in vacuum. The mixture was extracted with ethyl acetate (70 mL × 2). The combined organic layers were dried over sodium sulfate, filtered and concentrated under reduced pressure. The crude product was purified by flash column (ISCO 40 g silica, 0-20% ethyl acetate in petroleum ether, gradient over 20 min) to give 1-(5-chloropyridin-3-yl)ethan-1-one (2.5 g, 16.07 mmol, 77%) as a white solid. ¹H NMR (400 MHz, Chloroform-d) δ 9.03 (d, J = 1.8 Hz, 1H), 8.75 (d, J = 2.3 Hz, 1H), 8.21 (t, J = 2.1 Hz, 1H), 2.65 (s, 3H).

### Step 2: Preparation of 2-Bromo-1-(5-Chloropyridin-3-yl)Ethan-1-One

To a solution of 1-(5-chloropyridin-3-yl)ethan-1-one (1 g, 6.43 mmol) in acetic acid (10 mL) was added HBr (7.88 g, 32.14 mmol, 33% purity) and bromine (1.08 g, 6.75 mmol) at 0° C. The mixture was stirred at 25° C. for 0.5 h, filtered and the solids were dried in vacuo to afford 2-bromo-1-(5-chloropyridin-3-yl)ethan-1-one (2 g, crude) as a white solid.

### Step 3: Preparation of 1-(5-Chloro-3-Pyridyl)-2-(Propylamino)Ethanol

To a solution of 2-bromo-1-(5-chloropyridin-3-yl)ethan-1-one (1.5 g, 6.40 mmol) in ethanol (30 mL) was added sodium borohydride (1.21 g, 31.99 mmol) at 0° C. The mixture was stirred at 25° C. for 2 h and filtered. To the filtrate was added propan-1-amine (378 mg, 6.40 mmol) and the mixture was stirred at 80° C. for 2 h and concentrated. The crude product 1-(5-chloro-3-pyridyl)-2-(propylamino)ethanol (5 g, crude) was used in the next step without further purification. LCMS (ESI) m/z: 215.1 [M+H]⁺.

### Step 4: Preparation of N-[2-(5-Chloro-3-Pyridyl)-2-Hydroxy-Ethyl]-2-(3,4-Dichlorophenyl)-N-Propyl-Acetamide

To a solution of 2-(3,4-dichlorophenyl)acetic acid (955 mg, 4.66 mmol) in dimethylformamide (20 mL) were added N-(3-dimethylaminopropyl)-N′-ethylcarbodiimide (2.14 g, 11.18 mmol), 1-hydroxybenzotriazole (1.51 g, 11.18 mmol), N-methylmorpholine (2.83 g, 27.95 mmol) and 1-(5-chloro-3-pyridyl)-2-(propylamino)ethanol (2 g, 9.32 mmol). The mixture was stirred at 25° C. for 2 h and concentrated. The residue was purified by prep-HPLC (Kromasil C18 (250 × 50 mm × 10 um) column; 40-60% acetonitrile in a 10 mM ammonium bicarbonate solution in water, 10 min gradient) to give N-[2-(5-chloro-3-pyridyl)-2-hydroxy-ethyl]-2-(3,4-dichlorophenyl)-N-propyl-acetamide (98 mg, 242 µmol, 3%) as a yellow thick oil. ¹H NMR (400 MHz, Chloroform-d) δ 8.57 - 8.38 (m, 2H), 7.75 (t, J = 1.8 Hz, 1H), 7.43 (d, J = 8.2 Hz, 1H), 7.39 - 7.35 (m, 1H), 7.11 (dd, J = 2.0, 8.3 Hz, 1H), 5.04 (s, 1H), 5.02 - 4.98 (m, 1H), 4.44 - 4.30 (m, 1H), 3.78 - 3.58 (m, 3H), 3.52 (dd, J = 2.4, 14.4 Hz, 1H), 3.33 - 3.04 (m, 2H), 1.62 - 1.48 (m, 2H), 0.92 (t, J = 7.4 Hz, 3H); LCMS (ESI) m/z: 400.9 [M+H]⁺.

Compound 46 was synthesized according to the protocol described for compound 37:

| Name | Structure | NMR, MS | Compd# |
|---|---|---|---|
| N-[2-(5-bromo-3-pyridyl)-2-hydroxyethyl]-N-propyl-2-[6-(trifluoromethyl)-3-pyridyl]acetamide | 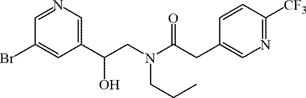 [SMILES: O=C(O)Cc1ccc2ncsc2c1 ; CCCNC[C@@H](O)c1cccnc1 ; "HBTU, DIPEA, DMF" ; CCCN(C[C@@H](O)c1cccnc1)C(=O)Cc1ccc2ncsc2c1] | ¹H NMR (400 MHz, Chloroform-d) δ 8.59 (d, J = 2.0 Hz, 2H), 8.49 (d, J = 1.5 Hz, 1H), 7.98 - 7.88 (m, 1H), 7.86 - 7.77 (m, 1H), 7.74 - 7.59 (m, 1H), 5.10 - 4.93 (m, 1H), 4.59 (br. s, 1H), 3.81 (s, 2H), 3.78 - 3.62 (m, 1H), 3.53 (dd, J = 2.4, 14.3 Hz, 1H), 3.40 - 3.28 (m, 1H), 3.27 - 3.05 (m, 1H), 1.74 - 1.54 (m, 2H), 1.03 - 0.86 (m, 3H); LCMS (ESI) m/z: 446.1 [M+H]⁺. | 46 |

### Example 19: Preparation of N-[(2S)-2-Hydroxy-2-(3-Pyridyl)ethyl]-N-Propyl-2-[6-(Trifluoromethyl)Pyridazin-3-yl]Acetamide (Compound 38)

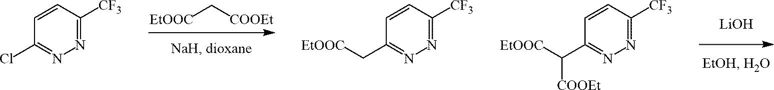 [SMILES: CCCNCC(O)c1cnccn1 ; "HBTU, DIEA, DMF" ; O=C(O)C1Cc2cc(Cl)c(Cl)cc2C1 ; CCCN(CC(O)c1cnccn1)C(=O)C1Cc2cc(Cl)c(Cl)cc2C1 ; "SFC"]

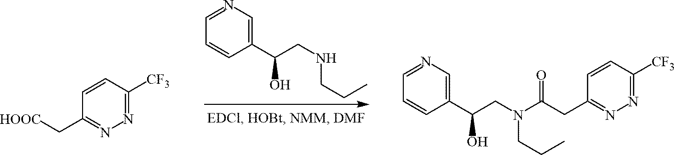

### Step 1: Preparation of Diethyl 2-[6-(Trifluoromethyl)Pyridazin-3-yl]Propanedioate

To a solution of diethyl propanedioate (1.32 g, 8.22 mmol) in dioxane (35 mL) was added sodium hydride (438 mg, 10.96 mmol, 60% purity) at 0° C. The reaction mixture was then stirred at 0° C. for 1h before 3-chloro-6-(trifluoromethyl)pyridazine (1 g, 5.48 mmol) was added. The resultant mixture was stirred at 110° C. for 16 h, quenched by the addition water (10 mL) and the aqueous layer was extracted with ethyl acetate (30 mL × 2). The combined organic layers were washed with brine (10 mL × 1), dried over sodium sulfate, filtered and concentrated under reduced pressure. The crude product was purified by flash column (ISCO 40 g silica, 10-20% ethyl acetate in petroleum ether, gradient over 20 min) to give ethyl 2-[6-(trifluoromethyl) pyridazin-3-yl]acetate (340 mg, crude) and diethyl 2-[6-(trifluoromethyl)pyridazin-3-yl]propanedioate (450 mg, 1.25 mmol, 23%) as a pale yellow oil. LCMS (ESI) m/z: 235.0 [M+H]⁺. LCMS (ESI) m/z: 307.0 [M+H]⁺.

### Step 2: Preparation of 2-[6-(Trifluoromethyl)Pyridazin-3-yl]Acetic Acid

To a solution of diethyl 2-[6-(trifluoromethyl)pyridazin-3-yl]propanedioate (430 mg, 1.40 mmol) in methanol (12 mL) and water (4 mL) was added lithium hydroxide hydrate (177 mg, 4.21 mmol). The mixture was stirred at 25° C. for 24 h and acidified with 2N HCl to pH 5~6 at 0° C., and then concentrated in vacuum. The residue was purified by prep-HPLC (Waters Xbridge BEH C18 100 × 25 mm × 5 um column; 1-20% acetonitrile in a 10 mM ammonium bicarbonate solution in water, 8 min gradient) to give 2-[6-(trifluoromethyl)pyridazin-3-yl]acetic acid (70 mg, 340 µmol, 24%) as a white solid. LCMS (ESI) m/z: 207.1 [M+H]⁺. ¹H NMR (400 MHz, Dimethylsulfoxide-d₆) δ 8.04 (d, J = 8.7 Hz, 1H), 7.88 - 7.82 (m, 1H), 3.69 - 3.60 (m, 2H)

### Step 3: Preparation of N-[(2S)-2-Hydroxy-2-(3-Pyridyl)ethyl]-N-Propyl-2-[6-(Trifluoromethyl)Pyridazin-3-yl]Acetamide

To a solution of 2-[6-(trifluoromethyl)pyridazin-3-yl]acetic acid (70 mg, 340 µmol) in dimethylformamide (2 mL) were added N-(3-dimethylaminopropyl)-N′-ethylcarbodiimide (78 mg, 408 µmol), HOBT (55 mg, 408 µmol), N-methylmorpholine (103 mg, 1.02 mmol, 112 µL) and (1S)-2-(propylamino)-1-(3-pyridyl)ethanol (61 mg, 340 µmol). The mixture was stirred at 25° C. for 2 h and concentrated. The resultant residue was purified by prep-HPLC (Waters Xbridge BEH C18 100 × 30 mm × 10 um column; 20-45% acetonitrile in a 10 mM ammonium bicarbonate solution in water, 10 min gradient) to give N-[(2S)-2-hydroxy-2-(3-pyridyl)ethyl]-N-propyl-2-[6-(trifluoromethyl)pyridazin-3-yl]acetamide (6 mg, 17 µmol, 5%) as a pale yellow thick oil. ¹H NMR (400 MHz, Chloroform-d) δ 8.68 -8.55 (m, 1H), 8.51 (dd, J = 1.4, 4.8 Hz, 1H), 7.89 - 7.77 (m, 2H), 7.76 - 7.64 (m, 1H), 7.39 - 7.23 (m, 1H), 5.21 - 4.93 (m, 1H), 4.69 - 4.36 (m, 1H), 4.34 - 3.84 (m, 2H), 3.72 - 3.55 (m, 2H), 3.51 - 3.10 (m, 2H), 1.69 - 1.46 (m, 2H), 1.04 - 0.78 (m, 1H); LCMS (ESI) m/z: 369.0 [M+H]⁺.

### Example 20: Preparation of 2-(6-Cyano-3-Pyridyl)-N-[(2S)-2-Hydroxy-2-(3-Pyridyl)ethyl]-N-Propyl-Acetamide (Compound 39)

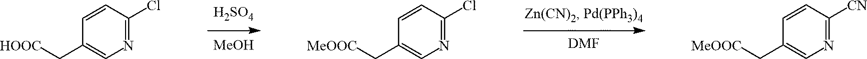

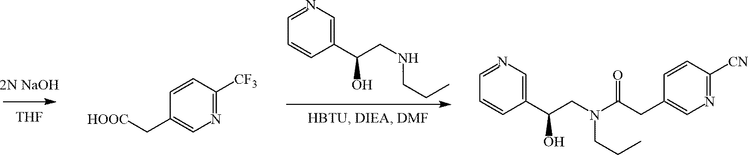

### Step 1: Preparation of Methyl 2-(6-Chloro-3-Pyridyl)Acetate

To a solution of 2-(6-chloro-3-pyridyl)acetic acid (2 g, 11.66 mmol) in methanol (20 mL) was added sulfuric acid (1.49 g, 15.15 mmol). The mixture was stirred at 25° C. for 1 h and concentrated to dryness. The resultant crude product was treated with saturated sodium carbonate (30 mL) and then extracted with ethyl acetate (30 mL × 2). The combined organic layers were washed with brine (30 mL), dried over sodium sulfate, filtered and concentrated to dryness to give methyl 2-(6-chloro-3-pyridyl)acetate (1.88 g, 10.13 mmol, 87%) as a pale red oil. The material was used directly in the next step without purification. ¹H NMR (400 MHz, Chloroform-d) δ 8.29 (d, J = 2.3 Hz, 1H), 7.62 (dd, J = 2.6, 8.2 Hz, 1H), 7.31 (d, J = 8.2 Hz, 1H), 3.72 (s, 3H), 3.62 (s, 2H).

### Step 2: Preparation of Methyl 2-(6-Cyano-3-Pyridyl)Acetate

To a solution of methyl 2-(6-chloro-3-pyridyl)acetate (1.7 g, 9.16 mmol) in dimethylformamide (20 mL) were added zinc cyanide (1.08 g, 9.16 mmol) and palladium-tetrakis(triphenylphosphine) (529 mg, 458 µmol). The mixture was stirred at 100° C. for 12 h. Water (20 mL) was added to the reaction and the aqueous phase was extracted with ethyl acetate (30 mL × 2). The combined organic layers were washed with brine (15 mL), dried over sodium sulfate, filtered and concentrated to dryness. The crude product was purified by flash column (ISCO 20 g silica, 0-30% ethyl acetate in petroleum ether, gradient over 20 min) to give methyl 2-(6-cyano-3-pyridyl)acetate (350 mg, 1.99 mmol, 22%) as a colorless oil. Recovered methyl 2-(6-chloro-3-pyridyl)acetate (1.1 g, 5.93 mmol, 65%) was recycled as a colorless oil. ¹H NMR (400 MHz, Chloroform-d) δ 8.64 (d, J = 1.7 Hz, 1H), 7.81 (dd, J = 2.2, 8.1 Hz, 1H), 7.69 (d, J = 7.9 Hz, 1H), 3.75 (s, 3H), 3.74 (s, 2H).

### Step 3: Preparation of 2-(6-Cyano-3-Pyridyl)Acetic Acid

To a solution of methyl 2-(6-cyano-3-pyridyl)acetate (250 mg, 1.42 mmol) in tetrahydrofuran (5 mL) was added sodium hydroxide (2 M, 851 µL). The reaction mixture was stirred at 25° C. for 1 h and then was acidified with acetic acid until pH =4. The aqueous layer was extracted with ethyl acetate (30 mL × 2). The organic layer was washed with brine (20 mL), dried over sodium sulfate, filtered and concentrated to give 2-(6-cyano-3-pyridyl)acetic acid (130 mg, crude) as a pale pink solid. The material was used directly in the next step without additional purification.

### Step 4: Preparation of 2-(6-Cyano-3-Pyridyl)-N-[(2S)-2-Hydroxy-2-(3-Pyridyl)Ethyl]-N-Propyl-Acetamide

To a solution of 2-(6-cyano-3-pyridyl)acetic acid (130 mg, 802 µmol) in dimethylformamide (3 mL) were added (1S)-2-(propylamino)-1-(3-pyridyl)ethanol (145 mg, 802 µmol), N,N,N′,N′-tetramethyl-O-(1H-benzotriazol-1-yl)uronium hexafluorophosphate (334 mg, 882 µmol) and diisopropylethylamine (311 mg, 2.41 mmol). The mixture was stirred at 20° C. for 1 h and filtered. The filtrate was purified by prep-HPLC (Phenomenex Gemini-NX C18 75 × 30 3u column; 8-28% acetonitrile in an a 10 mM ammonium bicarbonate solution in water, 7 min gradient) to give 2-(6-cyano-3-pyridyl)-N-[(2S)-2-hydroxy-2-(3-pyridyl)ethyl]-N-propyl-acetamide (116 mg, 357 µmol, 45%) as a pale yellow thick oil. ¹H NMR (400 MHz, Chloroform-d) δ 8.66 - 8.56 (m, 2H), 8.52 (dd, J = 1.5, 4.9 Hz, 1H), 7.82 - 7.72 (m, 2H), 7.71 - 7.62 (m, 1H), 7.38 - 7.27 (m, 1H), 5.11 - 4.96 (m, 1H), 4.08 (d, J = 15.9 Hz, 0.2H), 3.81 (s, 1.5H), 3.73 - 3.66 (m, 1H), 3.59 - 3.51 (m, 1H), 3.39 - 3.10 (m, 2H), 1.70 - 1.59 (m, 2H), 1.00 - 0.86 (m, 3H); LCMS (ESI) m/z: 325.2 [M+H]⁺.

### Example 21: Preparation of 2-(1,3-Benzothiazol-6-yl)-N-[(2S)-2-Hydroxy-2-(3-Pyridyl)Ethyl]-N-Propyl-Acetamide (Compound 40)

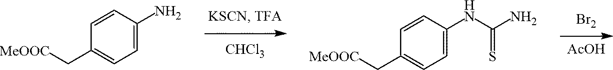

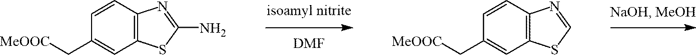

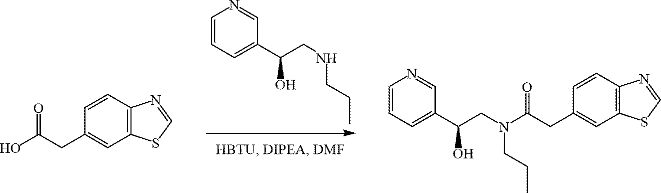

### Step 1: Preparation of Methyl 2-[4-(Carbamothioylamino)Phenyl]acetate

A mixture of methyl 2-(4-aminophenyl)acetate (2.53 g, 15.32 mmol), KSCN (2.23 g, 22.97 mmol) and trifluoroacetic acid (4.37 g, 38.29 mmol, 2.83 mL) in chloroform (40 mL) was stirred at 80° C. for 12 h. The reaction mixture was treated with chloroform (30 mL) and then washed with water (15 mL), dried over sodium sulfate, filtered and concentrated. The crude product was purified by flash column (ISCO 20 g silica, 40-80% ethyl acetate in petroleum ether, gradient over 40 min) to give methyl 2-[4-(carbamothioylamino)phenyl]acetate (1.35 g, 6.02 mmol, 39%) as a pale yellow solid. ¹H NMR (400 MHz, Dimethylsulfoxide-d₆) δ 9.65 (s, 1H), 7.34 (d, J = 8.4 Hz, 2H), 7.21 (d, J = 8.4 Hz, 2H), 3.64 (s, 2H), 3.61 (s, 3H)

### Step 2: Preparation of Methyl 2-(2-Amino-1,3-Benzothiazol-6-yl)Acetate

To a solution of methyl 2-[4-(carbamothioylamino)phenyl]acetate (1.2 g, 5.35 mmol) in acetic acid (24 mL) was added bromine (1.03 g, 6.42 mmol) and the mixture was stirred at 80° C. for 2 h. The reaction mixture was concentrated to dryness. The residue was treated with saturated sodium thiosulfate (10 mL) and saturated aqueous solution of sodium carbonate (30 mL) and the aqueous layer was extracted with ethyl acetate (30 mL × 2). The combined organic layers were washed with brine (15 mL), dried over sodium sulfate, filtered and concentrated to dryness. The crude product was purified by flash column (ISCO 12 g silica, 10-50% ethyl acetate in petroleum ether, gradient over 20 min) to yield methyl 2-(2-amino-1,3-benzothiazol-6-yl)acetate (0.55 g, 2.47 mmol, 46%) as a pale yellow solid. ¹H NMR (400 MHz, Dimethylsulfoxide-d₆) δ 7.53 (d, J = 1.6 Hz, 1H), 7.42 (s, 2H), 7.26 (d, J = 8.1 Hz, 1H), 7.08 (dd, J = 1.8, 8.2 Hz, 1H), 3.66 (s, 2H), 3.60 (s, 3H)

### Step 3: Preparation of Methyl 2-(1,3-Benzothiazol-6-yl)Acetate

To a solution of methyl 2-(2-amino-1,3-benzothiazol-6-yl)acetate (0.53 g, 2.38 mmol) in tetrahydrofuran (15 mL) was added isopentyl nitrite (559 mg, 4.77 mmol, 642 µL). The reaction mixture was stirred at 70° C. for 2 h. The reaction mixture was concentrated to dryness and the crude product was purified by flash column (ISCO 12 g silica, 0-20% ethyl acetate in petroleum ether, gradient over 20 min) to afford methyl 2-(1,3-benzothiazol-6-yl)acetate (0.41 g, 1.96 mmol, 82%) as a pale yellow oily liquid. ¹H NMR (400 MHz, Chloroform-d) δ 8.98 (s, 1H), 8.10 (d, J = 8.4 Hz, 1H), 7.90 (d, J = 1.1 Hz, 1H), 7.45 (dd, J = 1.7, 8.4 Hz, 1H), 3.80 (s, 2H), 3.73 (s, 3H)

### Step 4: Preparation of 2-(1,3-Benzothiazol-6-yl)Acetic Acid

To a solution of methyl 2-(1,3-benzothiazol-6-yl)acetate (0.39 g, 1.86 mmol) in methanol (8 mL), was added sodium hydroxide (2 M, 1.86 mL). The reaction mixture was stirred at 25° C. for 1 h and then it was treated with water (3 mL). The solution was then acidified with 1M HCl (5 mL) at 0° C. and filtered. The solids were dried to obtain 2-(1,3-benzothiazol-6-yl)acetic acid (0.31 g, 1.58 mmol, 85%) as a pale yellow solid, and used directly in the next step without further purification.

### Step 5: 2-(1,3-Benzothiazol-6-yl)-N-[(2S)-2-Hydroxy-2-(3-Pyridyl)Ethyl]-N-Propyl-Acetamide

To a solution of 2-(1,3-benzothiazol-6-yl)acetic acid (100 mg, 518 µmol) in dimethylformamide (2 mL), were added (1S)-2-(propylamino)-1-(3-pyridyl)ethanol (93 mg, 518 µmol), N,N,N′,N′-tetramethyl-O-(1H-benzotriazol-1-yl)uronium hexafluorophosphate (216 mg, 569 µmol) and diisopropylethylamine (201 mg, 1.55 mmol, 270 µL). The reaction mixture was stirred at 25° C. for 1 h and then it was filtered. The filtrate was purified by prep-HPLC (Phenomenex Gemini-NX C18 75 × 30 mm × 3 um column; 15-35% acetonitrile in a 10 mM ammonium bicarbonate solution in water, 7 min gradient) to yield 2-(1,3-benzothiazol-6-yl)-N-[(2S)-2-hydroxy-2-(3-pyridyl)ethyl]-N-propyl-acetamide (96 mg, 269 µmol, 52%) as a pale yellow thick oil. ¹H NMR (400 MHz, Chloroform-d) δ 8.99 (s, 1H), 8.64 - 8.48 (m, 2H), 8.11 (d, J = 8.4 Hz, 1H), 7.93 - 7.81 (m, 1H), 7.72 (br. d, J = 7.9 Hz, 1H), 7.41 (dd, J = 1.5, 8.4 Hz, 1H), 7.22 (dd, J = 5.1, 7.7 Hz, 1H), 5.18 - 4.86 (m, 2H), 3.90 (s, 2H), 3.82 - 3.49 (m, 2H), 3.38 - 3.05 (m, 2H), 1.64 - 1.50 (m, 2H), 0.90 (t, J = 7.4 Hz, 3H); LCMS (ESI) m/z: 356.2 [M+H]⁺.

### Example 22: Preparation of Enantiomer 1 (Compound 41) and Enantiomer 2 (Compound 42) of 5,6-Dichloro-N-(2-Hydroxy-2-Pyrazin-2-yl-Ethyl)-N-Propyl-Indane-2-Carboxamide

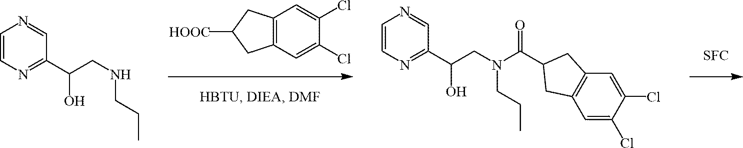

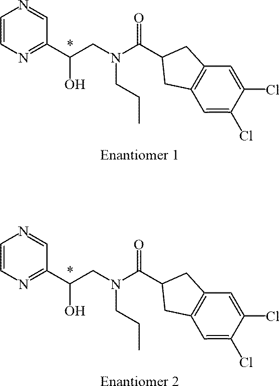

To a solution of 2-(propylamino)-1-pyrazin-2-yl-ethanol (190 mg, 1.05 mmol) in dimethylformamide (4 mL) was added 5,6-dichloroindane-2-carboxylic acid (242 mg, 1.05 mmol), N,N,N′,N′-tetramethyl-O-(1H-benzotriazol-1-yl)uronium hexafluorophosphate (437 mg, 1.15 mmol) and diisopropylethylamine (406 mg, 3.15 mmol). The mixture was stirred at 20° C. for 1 h, filtered and the filtrate was purified directly by prep-HPLC (Waters Xbridge BEH C18 100 × 30 10 um column; 30-60% acetonitrile in an a 10 mM ammonium bicarbonate solution in water, 10 min gradient) to obtain 5,6-dichloro-N-(2-hydroxy-2-pyrazin-2-yl-ethyl)-N-propyl-indane-2-carboxamide (180 mg) as a white solid. An amount of 120 mg was chirally separated by preparative SFC (Phenomenex-Cellulose-2 (250 mm × 30 mm, 10 um) column, 40° C., eluting with 38% ethanol containing 0.1% ammonium hydroxide in a flow of 70 g/min CO2 at 100 bar) to obtain enantiomer 1 (12 mg, 29 µmol, 3%) and enantiomer 2 (62 mg, 158 µmol, 15%) as white solids.

NMR and MS of the racemate: ¹H NMR (400 MHz, Chloroform-d) δ 8.88 (d, J = 1.0 Hz, 1H), 8.58 - 8.47 (m, 2H), 7.30 - 7.27 (m, 2H), 5.36 (d, J = 4.9 Hz, 1H), 5.14 - 5.04 (m, 1H), 3.90 - 3.72 (m, 2H), 3.60 - 3.46 (m, 1H), 3.39 - 3.25 (m, 2H), 3.24 - 3.13 (m, 2H), 3.13 - 3.00 (m, 2H), 1.72 - 1.62 (m, 2H), 0.93 (t, J = 7.4 Hz, 3H); LCMS (ESI) m/z: 394.1 [M+H]⁺.

Compound 41: ¹H NMR (400 MHz, Chloroform-d): δ 8.89 (d, J = 1.0 Hz, 1H), 8.55 - 8.49 (m, 2H), 7.29 (s, 1H), 7.28 - 7.27 (m, 1H), 5.37 (d, J = 4.9 Hz, 1H), 5.13 - 5.06 (m, 1H), 3.89 - 3.77 (m, 2H), 3.55 (quin, J = 8.3 Hz, 1H), 3.39 - 3.25 (m, 2H), 3.23 - 3.15 (m, 2H), 3.12 - 3.01 (m, 2H), 1.71 - 1.61 (m, 2H), 0.94 (t, J = 7.4 Hz, 3H); LCMS (ESI)m/z: 394.2 [M+H]⁺.

Compound 42: ¹H NMR (400 MHz, Chloroform-d): δ 8.89 (d, J = 1.0 Hz, 1H), 8.55 - 8.49 (m, 2H), 7.29 (s, 1H), 7.28 - 7.27 (m, 1H), 5.37 (d, J = 4.9 Hz, 1H), 5.13 - 5.06 (m, 1H), 3.89 - 3.77 (m, 2H), 3.55 (quin, J = 8.3 Hz, 1H), 3.39 - 3.25 (m, 2H), 3.23 - 3.15 (m, 2H), 3.12 - 3.01 (m, 2H), 1.71 - 1.61 (m, 2H), 0.94 (t, J = 7.4 Hz, 3H); LCMS (ESI) m/z: 394.2 [M+H]⁺.

### Example 23: Preparation of 2-(5-Chloro-3-Pyridyl)-N-[(2S)-2-Hydroxy-2-(3-Pyridyl)Ethyl]-N-Propyl-Acetamide (Compound 44)

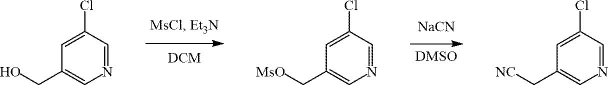

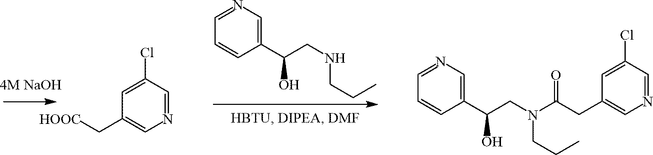

### Step 1: Preparation of (5-Chloro-3-Pyridyl)Methyl Methanesulfonate

To a solution of (5-chloro-3-pyridyl)methanol (1 g, 6.97 mmol) in dichloromethane (20 mL) was added triethylamine (1.06 g, 10.45 mmol) and methanesulfonyl chloride (957 mg, 8.36 mmol) at 0° C. The mixture was stirred at 25° C. for 1 h and then treated with dichloromethane (30 mL). The organic layer was washed with saturated aqueous solution of ammonium chloride (15 mL) and brine (15 mL). The combined organic layer was dried over sodium sulfate, filtered and concentrated to dryness to give (5-chloro-3-pyridyl)methyl methanesulfonate (1.4 g, crude) as a brown oil. The material was used directly in the next step without additional purification.

### Step 2: Preparation of 2-(5-Chloro-3-Pyridyl)Acetonitrile

To a solution of (5-chloro-3-pyridyl)methyl methanesulfonate (1.4 g, 6.32 mmol) in dimethylsulfoxide (20 mL) was added sodium cyanide (929 mg, 18.95 mmol). The mixture was stirred at 20° C. for 3 h before it was treated with water (30 mL). The aquous layer was extracted with ethyl acetate (30 mL × 2). The combined organic layers were washed with brine (15 mL), dried over sodium sulfate, filtered and concentrated to dryness. The crude product was purified by flash column (ISCO 10 g silica, 0-50% ethyl acetate in petroleum ether, gradient over 20 min) to give 2-(5-chloro-3-pyridyl)acetonitrile (0.4 g, 2.62 mmol, 42%) as a brown oil. ¹H NMR (400 MHz, Chloroform-d) δ 8.58 (d, J = 2.2 Hz, 1H), 8.48 (d, J = 1.5 Hz, 1H), 7.74 (t, J = 2.1 Hz, 1H), 3.80 (s, 2H).

### Step 3: Preparation of 2-(5-Chloro-3-Pyridyl)Acetic Acid

A solution of 2-(5-chloro-3-pyridyl)acetonitrile (0.2 g, 1.31 mmol) in sodium hydroxide (4 M, 1.64 mL) was stirred at 110° C. for 0.5 h. The reaction mixture was acidified with concentrated HCl until pH =1. Then the mixture was filtered. The filtrate was purified by prep-HPLC (Nano-micro Kromasil C18 80 × 25 3u column; 3-30% acetonitrile in an a 0.04% hydrochloric acid solution in water, 7 min gradient) to give 2-(5-chloro-3-pyridyl)acetic acid.HCl (140 mg, 673 µmol, 51%) as a red solid.

### Step 4: Preparation of 2-(5-Chloro-3-Pyridyl)-N-[(2S)-2-Hydroxy-2-(3-Pyridyl)ethyl]-N-Propyl-Acetamide

To a solution of (1S)-2-(propylamino)-1-(3-pyridyl)ethanol (104 mg, 577 µmol) in dimethylformamide (2 mL) were added 2-(5-chloro-3-pyridyl)acetic acid. HCl (120 mg, 577 µmol), N,N,N′,N′-tetramethyl-O-(1H-benzotriazol-1-yl)uronium hexafluorophosphate (241 mg, 634 µmol) and diisopropylethylamine (224 mg, 1.73 mmol). The mixture was stirred at 20° C. for 1 h and filtered. The filtrate was purified by prep-HPLC (Waters Xbridge Prep OBD C18 150 × 40 10 um column; 15-35% acetonitrile in an a 10 mM ammonium bicarbonate solution in water, 8 min gradient) to give 2-(5-chloro-3-pyridyl)-N-[(2S)-2-hydroxy-2-(3-pyridyl)ethyl]-N-propyl-acetamide (89 mg, 264 µmol, 46%) as a pale yellow thick oil. ¹H NMR (400 MHz, Chloroform-d) δ 8.68 - 8.42 (m, 3H), 8.40 - 8.25 (m, 1H), 7.81 - 7.72 (m, 1H), 7.70 - 7.59 (m, 1H), 7.39 - 7.28 (m, 1H), 5.10 - 4.91 (m, 1H), 4.55 (br. s, 1H), 3.97 - 3.60 (m, 3H), 3.55 (dd, J = 2.7, 14.3 Hz, 1H), 3.40 - 3.10 (m, 2H), 1.65 - 1.55 (m, 2H), 0.99 - 0.86 (m, 3H); LCMS (ESI) m/z: 334.2 [M+H]⁺.

### Example 24: Preparation of N-[(2S)-2-Hydroxy-2-(3-Pyridyl)Ethyl]-N-Propyl-2-[5-(Trifluoromethoxy)-2-Pyridyl]Acetamide (Compound 45)

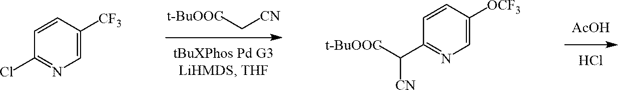

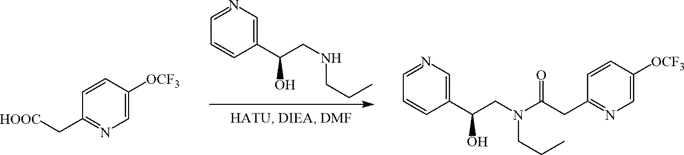

### Step 1: Preparation of Tert-butyl 2-Cyano-2-[5-(Trifluoromethoxy)-2-Pyridyl]Acetate

A solution of 2-chloro-5-(trifluoromethoxy)pyridine (1 g, 5.06 mmol), tert-butyl 2-cyanoacetate (1.79 g, 12.66 mmol, 1.81 mL), [2-(2-aminophenyl)phenyl]-methylsulfonyloxy-palladium;ditert-butyl-[2-(2,4,6-triisopropylphenyl)phenyl]phosphane (402 mg, 506 µmol) in tetrahydrofuran (15 mL) was degassed and then cooled to 0° C. Lithium bis(trimethylsilyl)amide (1 M, 15.19 mL) was added dropwise, then the mixture was slowly allowed to warm up to 25° C. for 16 h under N₂. The reaction mixture was treated with water ( 10 mL) and the aqueous solution was extracted with ethyl acetate(10 mL × 3). The combined organic phase was washed with brine (10 mL × 3), dried with anhydrous sodium sulfate, filtered and concentrated in vacuum. The crude product was purified by flash column (ISCO 10 g silica, 0-15% ethyl acetate in petroleum ether, gradient over 10 min) to give tert-butyl 2-cyano-2-[5-(trifluoromethoxy)-2-pyridyl]acetate (2 g, crude) as a yellow solid. LCMS (ESI) m/z: 303.2 [M+H]⁺.

### Step 2: Preparation of 2-[5-(Trifluoromethoxy)-2-Pyridyl]acetic Acid

A solution of tert-butyl 2-cyano-2-[5-(trifluoromethoxy)-2-pyridyl]acetate (1.9 g, 6.29 mmol) in acetic acid (10 mL) and HCl (15 mL) was heated at 100° C. for 3 h. The reaction mixture was concentrated under reduced pressure. The crude product was treated with ethyl acetate (5 mL) and then filtered. The filtrate was concentrated under reduced pressure to give a crude 2-[5-(trifluoromethoxy)-2-pyridyl]acetic acid.HCl (350 mg, 1.22 mmol, 19%) as a pale yellow solid. ¹H NMR (400 MHz, Chloroform-d) δ 8.51 (s, 1H), 7.67 (br. d, J = 8.3 Hz, 1H), 7.39 (d, J = 8.8 Hz, 1H), 3.96 (s, 2H); LCMS (ESI) m/z: 222.1 [M+H]⁺.

### Step 3: Preparation of N-[(2S)-2-Hydroxy-2-(3-Pyridyl)ethyl]-N-Propyl-2-[5-(Trifluoromethoxy)-2-Pyridyl]acetamide

To a solution of 2-[5-(trifluoromethoxy)-2-pyridyl]acetic acid.HCl (147 mg, 572 µmol) in dimethylformamide (3 mL) were added HATU (316 mg, 832 µmol),diisopropylethylamine (287 mg, 2.22 mmol) and (1S)-2-(propylamino)-1-(3-pyridyl)ethanol (100 mg, 555 µmol). The mixture was stirred at 25° C. for 2 h and filtered. The filtrate was purified by prep-HPLC (Waters Xbridge 150 × 25 5 um column; 15%-55% acetonitrile in an a 10 mM ammonium bicarbonate solution, 8 min gradient) to yield N-[(2S)-2-hydroxy-2-(3-pyridyl)ethyl]-N-propyl-2-[5-(trifluoromethoxy)-2-pyridyl]acetamide (28 mg, 72 µmol, 13%) as a pale yellow oil. ¹H NMR (400 MHz, Dimethylsulfoxide-d₆) δ 8.69 - 8.40 (m, 3H), 7.90 - 7.63 (m, 2H), 7.40 (br. s, 2H), 5.77 - 5.26 (m, 1H), 4.89 (br. s, 1H), 3.91 (s, 2H), 3.79 - 3.44 (m, 2H), 3.43 - 3.33 (m, 1H), 3.22 (br. s, 1H), 1.61 - 1.43 (m, 2H), 0.82 (m, 3H); LCMS (ESI) m/z: 384.1 [M+H]⁺.

### Example 25: Preparation of 2-(1,3-Benzothiazol-5-yl)-N-[(2S)-2-Hydroxy-2-(3-Pyridyl)Ethyl]-N-Propyl-Acetamide (Compound 47)

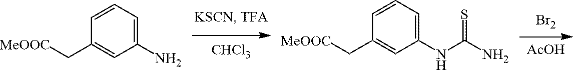

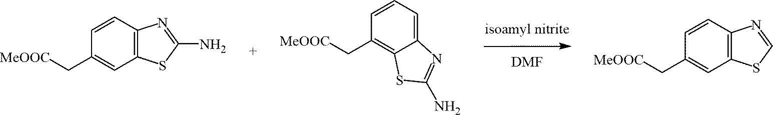 [SMILES: "MeMgBr" ; CCCN(CC(O)c1cncc(C(C)=O)c1)C(=O)Cc1ccc(C(F)(F)F)nc1 ; CCCN(CC(O)c1cncc(C(C)(C)O)c1)C(=O)Cc1ccc(C(F)(F)F)nc1 ; "THF"]

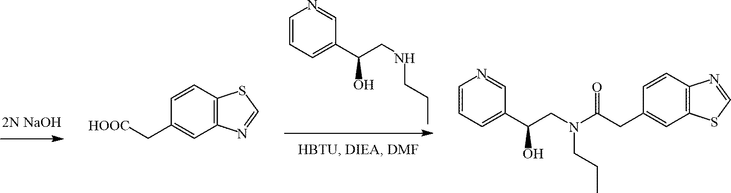

### Step 1: Preparation of Methyl 2-[3-(Carbamothioylamino)Phenyl]Acetate

To a solution of methyl 2-(3-aminophenyl)acetate (2.7 g, 16.34 mmol) in chloroform (40 mL) were added KSCN (2.38 g, 24.52 mmol) and trifluoroacetic acid (4.66 g, 40.86 mmol). The mixture was stirred at 80° C. for 12 h before water (30 mL) was added. The aqueous layer was extracted with ethyl acetate (60 mL × 2). The combined organic layers were washed with brine (30 mL), dried over sodium sulfate, filtered and concentrated to dryness. The crude product was purified by flash column (ISCO 20 g silica, 20-50% ethyl acetate in petroleum ether, gradient over 30 min) to give methyl 2-[3-(carbamothioylamino)phenyl]acetate (1 g, 4.46 mmol, 27%) as a yellow solid.

### Step 2: Preparation of Methyl 2-(2-Amino-1,3-Benzothiazol-5-yl)Acetate and Methyl 2-(2-Amino-1,3-Benzothiazol-7-yl)Acetate

To a solution of methyl 2-[3-(carbamothioylamino)phenyl]acetate (0.9 g, 4.01 mmol) in acetic acid (15 mL) was added bromine (770 mg, 4.82 mmol, 248 µL). The reaction mixture was stirred at 80° C. for 2 h and then concentrated to dryness. The crude residue was treated with saturated sodium thiosulfate (10 mL) and saturated aqueous solution of sodium carbonate (30 mL). The aqueous layer was extracted with ethyl acetate (30 mL × 2). The combined organic layers were washed with brine (15 mL),dried over sodium sulfate, filtered and concentrated to dryness. The crude product was purified by flash column (ISCO 20 g silica, 20-50% ethyl acetate in petroleum ether, gradient over 30 min) to give a mixture of methyl 2-(2-amino-1,3-benzothiazol-5-yl)acetate and methyl 2-(2-amino-1,3-benzothiazol-7-yl)acetate (0.42 g, 1.89 mmol, 47%) as a brown oil.

### Step 3: Preparation of Methyl 2-(1,3-Benzothiazol-7-yl)Acetate and Methyl 2-(1,3-Benzothiazol-5-yl)Acetate

A mixture of methyl 2-(2-amino-1,3-benzothiazol-5-yl)acetate and methyl 2-(2-amino-1,3-benzothiazol-7-yl)acetate (0.4 g, 1.80 mmol) in tetrahydrofuran (8 mL) was treated with isopentyl nitrite (422 mg, 3.60 mmol). The reaction mixture was stirred at 70° C. for 2 h and concentrated. The crude product was purified by flash column (ISCO 10 g silica, 8-12% ethyl acetate in petroleum ether, gradient over 30 min). methyl 2-(1,3-benzothiazol-7-yl)acetate (90 mg, 434 µmol, 24%) as a pale yellow oil and methyl 2-(1,3-benzothiazol-5-yl)acetate (100 mg, 483 µmol, 27%) as a pale yellow oil. ¹H NMR (400 MHz, Chloroform-d) for methyl 2-(1,3-benzothiazol-7-yl)acetate δ 9.02 (s, 1H), 8.09 (d, J = 8.2 Hz, 1H), 7.52 (t, J = 7.8 Hz, 1H), 7.38 (d, J = 7.3 Hz, 1H), 3.93 (s, 2H), 3.72 (s, 3H). ¹H NMR (400 MHz, Chloroform-d) for methyl 2-(1,3-benzothiazol-5-yl)acetate δ 9.01 (s, 1H), 8.06 (s, 1H), 7.93 (d, J = 8.2 Hz, 1H), 7.40 (d, J = 8.4 Hz, 1H), 3.82 (s, 2H), 3.73 (s, 3H).

### Step 4: Preparation of 2-(1,3-Benzothiazol-5-yl)Acetic Acid

To a solution of methyl 2-(1,3-benzothiazol-5-yl)acetate (90 mg, 434 µmol) in methanol (3 mL) was added sodium hydroxide (2 M, 434 µL). The reaction mixture was stirred at 25° C. for 1 h and then concentrated under reduced pressure to remove methanol. The crude residue was acidified with 2 M HCl until pH =1 and filtered. The solids were dried in vacuo and used directly without purification. 2-(1,3-benzothiazol-5-yl)acetic acid (75 mg, 388 µmol, 89%) was obtained as a white solid.

### Step 5: Preparation of 2-(1,3-Benzothiazol-5-yl)-N-[(2S)-2-Hydroxy-2-(3-Pyridyl)ethyl]-N-Propyl-Acetamide

To a solution of (1S)-2-(propylamino)-1-(3-pyridyl)ethanol (61 mg, 336 µmol) in dimethylformamide (2 mL) were added 2-(1,3-benzothiazol-5-yl)acetic acid (65 mg, 336 µmol), N,N,N′,N′-tetramethyl-O-(1H-benzotriazol-1-yl)uronium hexafluorophosphate (140 mg, 370 µmol) and diisopropylethylamine (130 mg, 1.01 mmol). The mixture was stirred at 20° C. for 1 h and filtered. The filtrate was purified by prep-HPLC (Phenomenex Luna C18 200 × 40 10 um column; 10-35% acetonitrile in an a 10 mM ammonium bicarbonate solution in water, 10 min gradient) to give 2-(1,3-benzothiazol-5-yl)-N-[(2S)-2-hydroxy-2-(3-pyridyl)ethyl]-N-propyl-acetamide (64 mg, 181 µmol, 54%) as a pale yellow thick oil. ¹H NMR (400 MHz, Chloroform-d) δ 9.02 (s, 1H), 8.63 - 8.54 (m, 1H), 8.50 (dd, J = 1.6, 4.8 Hz, 1H), 8.01 (s, 1H), 7.96 (d, J = 8.2 Hz, 1H), 7.72 (td, J = 1.8, 7.8 Hz, 1H), 7.45 - 7.31 (m, 1H), 7.22 (dd, J = 4.8, 7.8 Hz, 1H), 5.13 - 4.90 (m, 2H), 3.92 (s, 2H), 3.75 (dd, J = 8.1, 14.4 Hz, 1H), 3.54 (dd, J = 2.3, 14.4 Hz, 1H), 3.39 - 3.24 (m, 1H), 3.22 - 3.06 (m, 1H), 1.64 - 1.49 (m, 2H), 0.90 (t, J = 7.4 Hz, 3H); LCMS (ESI) m/z: 356.2 [M+H]⁺.

### Example 26: Preparation of N-[2-(5-Acetyl-3-Pyridyl)-2-Hydroxy-Ethyl]-N-Propyl-2-[6-(Trifluoromethyl)-3-Pyridyl]Acetamide (Compound 48)

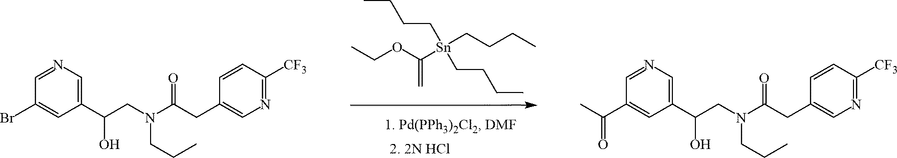

To a solution of N-[2-(5-bromo-3-pyridyl)-2-hydroxy-ethyl]-N-propyl-2-[6-(trifluoromethyl)-3-pyridyl]acetamide (50 mg, 112 µmol) in dimethylformamide (1 mL) were added tributyl(1-ethoxyvinyl)stannane (48 mg, 134 µmol) and Pd(PPh₃)₂Cl₂ (786 ug, 1 µmol) under N₂. The reaction mixture was stirred at 100° C. for 3 h and then cooled to 25° C. Then the mixture was treated with ethyl acetate (5 mL) and 40 mg KF in water (5 mL) and stirred at 25° C. for 1 h, then the layers were separated. The aqueous phase was extracted with acetate (10 mL × 3). The combined organic layers were washed with saturated sodium bicarbonate (10 mL), brine (10 mL), dried over sodium sulfate, filtered and concentrated in vacuum. The crude product was dissolved in tetrahydrofuran (2 mL) and HCl (2 M, 2 mL) was added, the mixture was stirred at 25° C. for 2 h. The reaction mixture was concentrated under reduced pressure. The crude product was purified by prep-HPLC (Waters Xbridge 150 × 25 5 um column; 25%-55% acetonitrile in an a 10 mM ammonium bicarbonate solution, 8 min gradient) to give N-[2-(5-acetyl-3-pyridyl)-2-hydroxy-ethyl]-N-propyl-2-[6-(trifluoromethyl)-3-pyridyl]acetamide (4 mg, 9 µmol, 8%) as a pale yellow thick oil. ¹H NMR (400 MHz, Dimethylsulfoxide-d₆) δ 9.15 - 9.04 (m, 1H), 8.86 - 8.77 (m, 1H), 8.60 - 8.52 (m, 1H), 8.31 - 8.23 (m, 1H), 7.88 - 7.78 (m, 1H), 7.73 - 7.60 (m, 1H), 5.18 - 5.02 (m, 1H), 4.63 (br. s, 1H), 3.82 (s, 2H), 3.75 (dd, J = 8.5, 14.4 Hz, 1H), 3.53 (dd, J = 2.6, 14.3 Hz, 1H), 3.40 - 3.19 (m, 2H), 2.64 (s, 3H), 1.72 - 1.65 (m, 2H), 1.01 - 0.86 (m, 3H); LCMS (ESI) m/z: 410.2 [M+H]⁺.

### Example 27: Preparation of 2-[4-(Difluoromethyl)-3-Fluoro-Phenyl]-N-[(2S)-2-Hydroxy-2-(3-Pyridyl)Ethyl]-N-Propyl-Acetamide (Compound 50)

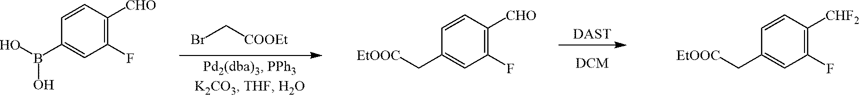

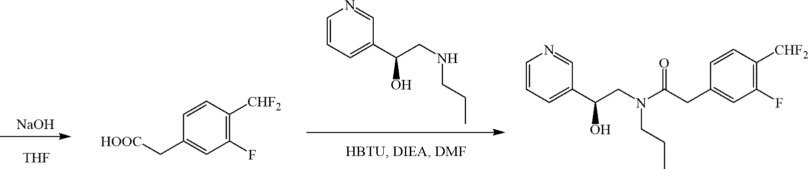

### Step 1: Preparation of Ethyl 2-(3-Fluoro-4-Formyl-Phenyl)Acetate

To a solution of (3-fluoro-4-formyl-phenyl)boronic acid (2.3 g, 13.70 mmol) in tetrahydrofuran (40 mL) /water (4 mL) were added ethyl 2-bromoacetate (1.83 g, 10.96 mmol), tris(dibenzylideneacetone) dipalladium(0) (251 mg, 274 µmol),triphenylphosphine (180 mg, 685 µmol) and potassium carbonate (3.79 g, 27.39 mmol). The mixture was stirred at 70° C. for 12 h. Water (30 mL) was added to the reaction and the aqueous layer was extracted with ethyl acetate (50 mL × 2). The combined organic layers were washed with brine (30 mL), dried over sodium sulfate, filtered and concentrated to dryness. The crude product was purified by flash column (ISCO 40 g silica, 0-10% ethyl acetate in petroleum ether, gradient over 20 min) to give ethyl 2-(3-fluoro-4-formyl-phenyl)acetate (750 mg, crude) as a pale yellow oil ¹H NMR (400 MHz, Chloroform-d) δ 10.34 (s, 1H), 7.84 (t, J = 7.6 Hz, 1H), 7.24 - 7.13 (m, 2H), 4.23 - 4.15 (m, 2H), 3.68 (s, 2H), 1.30 - 1.25 (m, 3H).

### Step 2: Preparation of Ethyl 2-[4-(Difluoromethyl)-3-Fluoro-Phenyl]Acetate

To a solution of ethyl 2-(3-fluoro-4-formyl-phenyl)acetate (750 mg, 3.57 mmol) in dichloromethane (10 mL) was added diethylaminosulfur trifluoride (1.15 g, 7.14 mmol). The mixture was stirred at 25° C. for 12 h before saturated solution of aqueous sodium bicarbonate (20 mL) was added to the reaction. The aqueous layer was extracted with dichloromethane (30 mL × 2). The combined organic layers were washed with brine (15 mL), dried over sodium sulfate, filtered and concentrated to dryness. The crude product was purified by flash column (ISCO 20 g silica, 0-10% ethyl acetate in petroleum ether, gradient over 30 min) to give ethyl 2-[4-(difluoromethyl)-3-fluoro-phenyl]acetate (600 mg, 2.58 mmol, 72%) as a pale yellow oil. ¹H NMR (400 MHz, Chloroform-d) δ 7.55 (t, J = 7.7 Hz, 1H), 7.17 (d, J = 7.9 Hz, 1H), 7.11 (d, J = 11.0 Hz, 1H), 7.03 - 6.73 (m, 1H), 4.18 (q, J = 7.1 Hz, 2H), 3.65 (s, 2H), 1.27 (t, J = 7.2 Hz, 3H).

### Step 3: Preparation of 2-[4-(Difluoromethyl)-3-Fluoro-Phenyl]Acetic Acid

To a solution of ethyl 2-[4-(difluoromethyl)-3-fluoro-phenyl]acetate (200 mg, 861 µmol) in tetrahydrofuran (5 mL) was added sodium hydroxide (2 M, 861 µL). The reaction mixture was stirred at 25° C. for 12 h and acidified with 2 M HCl until pH =4 and then the aqueous layer was extracted with ethyl acetate (30 mL × 2). The organic layer was washed with brine (20 mL), dried over sodium sulfate, filtered and concentrated to give 2-[4-(difluoromethyl)-3-fluoro-phenyl]acetic acid (150 mg, 735 µmol, 85%) as a pale yellow solid.

### Step 4: Preparation of 2-[4-(Difluoromethyl)-3-Fluoro-Phenyl]-N-[(2S)-2-Hydroxy-2-(3-Pyridyl)Ethyl]-N-Propyl-Acetamide

To a solution of (1S)-2-(propylamino)-1-(3-pyridyl)ethanol (100 mg, 555 µmol) in dimethylformamide (2 mL) was added 2-[4-(difluoromethyl)-3-fluoro-phenyl]acetic acid (113 mg, 555 µmol), N,N,N′,N′-tetramethyl-O-(1H-benzotriazol-1-yl)uronium hexafluorophosphate (231 mg, 610 µmol) and diisopropylethylamine (215 mg, 1.66 mmol, 290 µL). The mixture was stirred at 20° C. for 1 h and solution was filtered. The filtrate was purified by prep-HPLC (Waters Xbridge BEH C18 100 × 30 10 um column; 20-50% acetonitrile in an a 10 mM ammonium bicarbonate solution in water, 8 min gradient) to give 2-[4-(difluoromethyl)-3-fluoro-phenyl]-N-[(2S)-2-hydroxy-2-(3-pyridyl)ethyl]-N-propyl-acetamide (108 mg, 292 µmol, 53%) as a pale yellow thick oil. ¹H NMR (400 MHz, Chloroform-d) δ 8.65 - 8.57 (m, 1H), 8.53 (dd, J = 1.6, 4.8 Hz, 1H), 7.74 (td, J = 1.7, 7.8 Hz, 1H), 7.62 - 7.47 (m, 1H), 7.38 - 7.27 (m, 1H), 7.14 (d, J = 7.9 Hz, 1H), 7.08 (d, J = 11.0 Hz, 1H), 7.04 - 6.74 (m, 1H), 5.09 - 4.94 (m, 1H), 4.67 (br. s, 1H), 3.85 - 3.71 (m, 3H), 3.53 (dd, J = 2.6, 14.4 Hz, 1H), 3.35 - 3.06 (m, 2H), 1.60 - 1.50 (m, 2H), 0.92 (t, J = 7.4 Hz, 3H); LCMS (ESI) m/z: 367.2 [M+H]⁺.

### Example 28: Preparation of 2-(3-Chloro-4-Cyano-Phenyl)-N-(2-Hydroxy-2-Pyridazin-3-yl-Ethyl)-N-Propyl-Acetamide (Compound 52) and its Enantiomer 1 (Compound 53) and Enantiomer 2 (Compound 54)

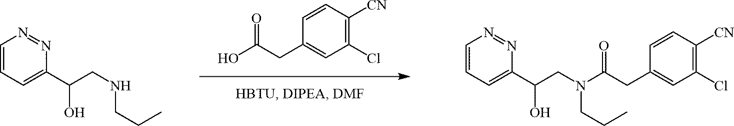

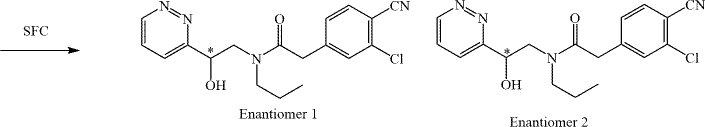

To a solution of 2-(3-chloro-4-cyano-phenyl)acetic acid (200 mg, 1.02 mmol) in dimethylformamide (3 mL) were added 2-(propylamino)-1-pyridazin-3-yl-ethanol (185 mg, 1.02 mmol), N,N,N′,N′-tetramethyl-O-(1H-benzotriazol-1-yl)uronium hexafluorophosphate (427 mg, 1.12 mmol) and diisopropylethylamine (396 mg, 3.07 mmol). The mixture was stirred at 20° C. for 1 h and filtered. The filtrate was purified by prep-HPLC (Waters Xbridge BEH C18 100 × 30 10 um column; 15-45% acetonitrile in an a 10 mM ammonium bicarbonate solution in water, 8 min gradient) to give 2-(3-chloro-4-cyanophenyl)-N-(2-hydroxy-2-pyridazin-3-yl-ethyl)-N-propyl-acetamide (160 mg) as a white solid. An amount of 150 mg of this product was chirally separated by preparative SFC Phenomenex-Cellulose-2 (250 mm x 30 mm,10 um) column, 40° C., eluting with 42% IPA containing 0.1% ammonium hydroxide in a flow of 70 g/min CO2 at 100 bar) to afford enantiomer 1 (12 mg, 32 µmol, 3%) as a white solid. and enantiomer 2 (55 mg, 152 µmol, 15%) as a pale yellow thick oil.

Compound 52: ¹H NMR (400 MHz, Chloroform-d) δ 9.11 (dd, J = 1.5, 4.9 Hz, 1H), 7.80 (dd, J = 1.3, 8.6 Hz, 1H), 7.64 (d, J = 7.9 Hz, 1H), 7.50 (dd, J = 4.9, 8.4 Hz, 1H), 7.39 (s, 1H), 7.23 (d, J = 7.7 Hz, 1H), 5.37 (d, J = 4.9 Hz, 1H), 5.28 - 5.19 (m, 1H), 3.99 - 3.83 (m, 2H), 3.76 (s, 2H), 3.44 - 3.27 (m, 2H), 1.74 -1.63 (m, 2H), 0.96 (t, J = 7.4 Hz, 3H); LCMS (ESI) m/z: 359.2 [M+H]⁺.

Compound 53: ¹H NMR (400 MHz, Chloroform-d) δ 9.11 (dd, J = 1.5, 4.9 Hz, 1H), 7.80 (dd, J = 1.3, 8.6 Hz, 1H), 7.64 (d, J = 7.9 Hz, 1H), 7.50 (dd, J = 4.9, 8.4 Hz, 1H), 7.39 (s, 1H), 7.23 (d, J = 7.7 Hz, 1H), 5.37 (d, J = 4.9 Hz, 1H), 5.28 - 5.19 (m, 1H), 3.99 - 3.83 (m, 2H), 3.76 (s, 2H), 3.44 - 3.27 (m, 2H), 1.74 -1.63 (m, 2H), 0.96 (t, J = 7.4 Hz, 3H); LCMS (ESI) m/z: 359.2 [M+H]⁺.

Compound 54: ¹H NMR (400 MHz, Chloroform-d) δ 9.10 (dd, J = 1.6, 4.9 Hz, 1H), 7.84 - 7.70 (m, 1H), 7.67 - 7.56 (m, 1H), 7.50 (dd, J = 5.0, 8.5 Hz, 1H), 7.44 - 7.37 (m, 1H), 7.26 - 7.19 (m, 1H), 5.38 (br. d, J = 4.8 Hz, 1H), 5.25 (td, J = 3.5, 6.8 Hz, 1H), 4.02 - 3.85 (m, 2H), 3.83 - 3.68 (m, 2H), 3.46 - 3.21 (m, 2H), 1.71 - 1.65 (m, 2H), 0.95 (t, J = 7.4 Hz, 3H); LCMS (ESI) m/z: 359.1 [M+H]⁺.

### Example 29: Preparation of N-[2-Hydroxy-2-[5-(1-Hydroxy-1-Methyl-Ethyl)-3-Pyridyl]Ethyl]-N-Propyl-2-[6-(Trifluoromethyl)-3-Pyridyl]Acetamide (Compound 55)

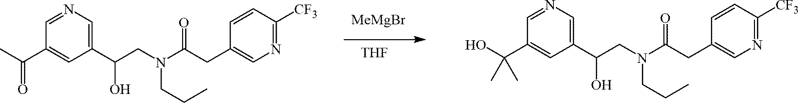

To a solution of N-[2-(5-acetyl-3-pyridyl)-2-hydroxy-ethyl]-N-propyl-2-[6-(trifluoromethyl)-3-pyridyl]acetamide (200 mg, 489 µmol) in tetrahydrofuran (3 mL) was added bromo(methyl)magnesium (1 M, 977 µL) at 0° C. The reaction mixture was stirred at 25° C. for 2 h and was quenched with water (1 mL). The reaction solution was concentrated under reduced pressure. The crude product was purified by prep-HPLC (Waters Xbridge 150 × 25 5 um column; 20%-45% acetonitrile in a 10 mM ammonium bicarbonate solution, 8 min gradient) to give 35 mg crude product. Then purified by prep-HPLC (Phenomenex Luna C18 200 × 40 mm × 10 um column ; 1%-50% acetonitrile in a 0.2% FA solution in water, 8 min gradient) to give N-[2-hydroxy-2-[5-(1-hydroxy-1-methyl-ethyl)-3-pyridyl]ethyl]-N-propyl-2-[6-(trifluoromethyl)-3-pyridyl]acetamide (14 mg, 30 µmol, 6%, FA) as a pale yellow thick oil. ¹H NMR (400 MHz, Methanol-d₄) δ 8.66 - 8.58 (m, 1H), 8.57 (s, 1H), 8.42 (d, J = 1.3 Hz, 1H), 8.08 - 7.96 (m, 1H), 7.87 (br. dd, J = 8.5, 14.7 Hz, 1H), 7.80 - 7.73 (m, 1H), 5.05 (td, J = 4.2, 8.4 Hz, 1H), 4.10 (d, J = 16.3 Hz, 1H), 4.01 - 3.87 (m, 1H), 3.87 - 3.79 (m, 1H), 3.71 (dd, J = 4.3, 13.6 Hz, 1H), 3.58 - 3.42 (m, 2H), 1.73 - 1.61 (m, 2H), 1.56 (d, J = 9.0 Hz, 6H), 0.93 (td, J = 7.4, 19.0 Hz, 3H); LCMS (ESI) m/z: 426.3 [M+H]⁺.

### Example 30: Preparation of N-[(1-Acetyl-4-Piperidyl)Methyl]-N-[(2S)-2-Hydroxy-2-(3-Pyridyl)Ethyl]-2-[6-(Trifluoromethyl)-3-Pyridyl]Acetamide (Compound 57)

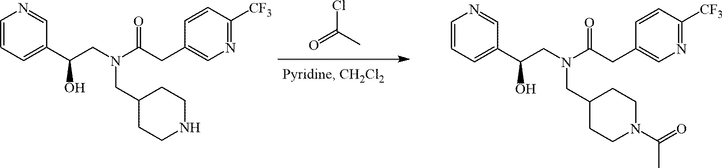

To a solution of N-[(2S)-2-hydroxy-2-(3-pyridyl)ethyl]-N-(4-piperidylmethyl)-2-[6-(trifluoromethyl)-3-pyridyl]acetamide.TFA (70 mg, 130 µmol) in dichloromethane (1 mL) were added pyridine (10 mg, 130 µmol) and acetyl chloride (10 mg, 130 µmol, 9 µL) at 0° C. The reaction mixture then was stirred at 25° C. for 12 h before it was filtered. The filtrated was purified by prep-HPLC (Waters Xbridge BEH C18 100 × 30 mm × 10 um column; 1-30% acetonitrile in an a 10 mM ammonium bicarbonate solution in water, 8 min gradient) to give N-[(1-acetyl-4-piperidyl)methyl]-N-[(2S)-2-hydroxy-2-(3-pyridyl)ethyl]-2-[6-(trifluoromethyl)-3-pyridyl]acetamide (18 mg, 38 µmol, 29%) as a white solid. ¹H NMR (400 MHz, Methanol-d₄) δ 8.69 - 8.42 (m, 3H), 8.03 - 7.75 (m, 3H), 7.51 - 7.37 (m, 1H), 5.06 (br. dd, J = 3.2, 8.8 Hz, 1H), 4.61 - 4.45 (m, 1H), 4.21 (d, J = 16.3 Hz, 1H), 4.03 - 3.83 (m, 3H), 3.68 - 3.41 (m, 3H), 3.26 (br. d, J = 13.1 Hz, 1H), 3.14 - 3.01 (m, 1H), 2.59 (br. t, J = 12.7 Hz, 1H), 2.16 - 1.99 (m, 4H), 1.85 - 1.61 (m, 2H), 1.35 - 1.03 (m, 2H); LCMS (ESI) m/z: 465.2 [M+H]⁺.

### Example 31: Preparation of N-[(2S)-2-Hydroxy-2-(3-Pyridyl)Ethyl]-N-[(1-Methyl-4-Piperidyl)Methyl]-2-[6-(Trifluoromethyl)-3-Pyridyl]Acetamide (Compound 58)

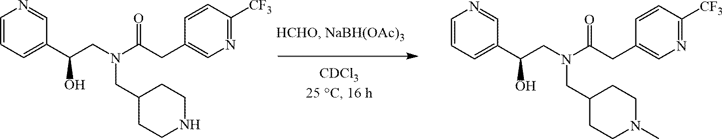

To a solution of N-[(2S)-2-hydroxy-2-(3-pyridyl)ethyl]-N-(4-piperidylmethyl)-2-[6-(trifluoromethyl)-3-pyridyl]acetamide.TFA (80 mg, 149 µmol) in chloroform (1 mL) were added formaldehyde (13 mg, 447 µmol) and triethylamine (15 mg, 149 µmol, 21 µL). The reaction mixture was stirred at 25° C. for 0.5 h and sodium triacetoxyborohydride (63 mg, 298 µmol) was added to the reaction mixture. The mixture was then stirred at 25° C. for 15 h and filtered. The filtrate was purified by prep-HPLC (Waters Xbridge BEH C18 100 × 30 mm × 10 um column; 10-40% acetonitrile in an a 10 mM ammonium bicarbonate solution in water, 8 min gradient) to give N-[(2S)-2-hydroxy-2-(3-pyridyl)ethyl]-N-[(1-methyl-4-piperidyl)methyl]-2-[6-(trifluoromethyl)-3-pyridyl]acetamide (1 mg, 21 µmol, 1%) as a white solid. ¹H NMR (400 MHz, Methanol-d₄) δ 8.70 - 8.39 (m, 3H), 8.02 - 7.73 (m, 3H), 7.52 - 7.36 (m, 1H), 5.05 (br. d, J = 7.3 Hz, 1H), 4.57 (br. s, 1H), 4.23 - 4.16 (m, 1H), 4.02 - 3.96 (m, 1H), 3.95 - 3.87 (m, 1H), 3.87 - 3.69 (m, 1H), 3.65 - 3.53 (m, 1H), 3.51 - 3.46 (m, 1H), 3.46 - 3.41 (m, 1H), 3.23 (br. dd, J = 7.3, 13.8 Hz, 1H), 2.93 (br. t, J = 10.9 Hz, 2H), 2.31 (s, 3H), 2.16 - 1.97 (m, 2H), 1.88 - 1.64 (m, 3H), 1.44 - 1.26 (m, 2H); LCMS (ESI) m/z: 437.2 [M+H]⁺.

### Example 32: Preparation of N-[(2S)-2-hydroxy-2-(3-pyridyl)ethyl]-N-propyl-2-[5-(trifluoromethyl)-3-pyridyl]acetamide (Compound 59).

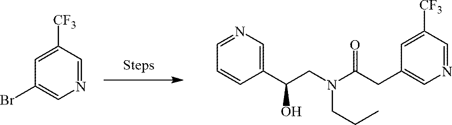

The compound 59 is synthesized similar to the protocol described for the compound 45. The compound N-[(2S)-2-hydroxy-2-(3-pyridyl)ethyl]-N-propyl-2-[5-(trifluoromethyl)-3-pyridyl]acetamide (68 mg, 182 µmol, 41%) was obtained as a viscous pale yellow oil. ¹H NMR (400 MHz, Methanol-d₄) δ 8.75 (br. d, J = 9.4 Hz, 1H), 8.67 (s, 1H), 8.65 - 8.54 (m, 1H), 8.52 - 8.41 (m, 1H), 8.05 - 7.83 (m, 2H), 7.51 -7.36 (m, 1H), 5.04 (dt, J = 3.9, 8.3 Hz, 1H), 4.22 - 3.90 (m, 2H), 3.89 - 3.81 (m, 1H), 3.74 - 3.56 (m, 1H), 3.55 - 3.43 (m, 2H), 1.75 - 1.55 (m, 2H), 1.00 - 0.85 (m, 3H); LCMS (ESI) m/z: 368.1 [M+H]⁺.

### Example 33: Preparation of N-Cyclobutyl-N-(2-Hydroxy-2-Pyridazin-3-yl-Ethyl)-2-[6-(Trifluoromethyl)-3-Pyridyl]acetamide (Compound 60) and its Enantiomer 1 (Compound 61) and Enantiomer 2 (Compound 62)

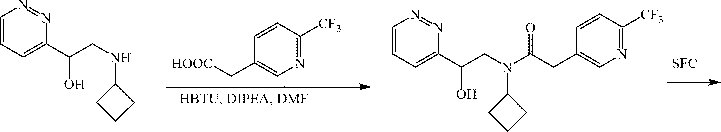

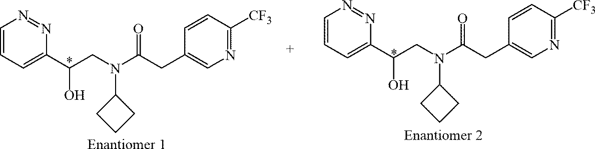

To a solution of 2-(cyclobutylamino)-1-pyridazin-3-yl-ethanol (200 mg, 1.03 mmol) in dimethylformamide (3 mL) were added 2-[6-(trifluoromethyl)-3-pyridyl]acetic acid (211 mg, 1.03 mmol), N,N,N′,N′-tetramethyl-O-(1H-benzotriazol-1-yl)uronium hexafluorophosphate (430 mg, 1.13 mmol) and diisopropylethylamine (399 mg, 3.09 mmol). The mixture was stirred at 20° C. for 1 h and filtered. The filtrate was purified by prep-HPLC (Kromasil C18 250 × 50 10 um column; 15-45% acetonitrile in an a 10 mM ammonium bicarbonate solution in water, 10 min gradient) to give N-cyclobutyl-N-(2-hydroxy-2-pyridazin-3-yl-ethyl)-2-[6-(trifluoromethyl)-3-pyridyl]acetamide (Compound 60, 180 mg) as a pale yellow thick oil.

Compound 60: ¹H NMR (400 MHz, Chloroform-d) δ 9.12 (dd, J = 1.4, 5.0 Hz, 1H), 8.59 (s, 1H), 7.87 -7.77 (m, 2H), 7.69 (d, J = 7.9 Hz, 1H), 7.51 (dd, J = 5.0, 8.5 Hz, 1H), 5.36 (br. s, 1H), 5.17 (dd, J = 2.1, 8.5 Hz, 1H), 4.38 (quin, J = 8.5 Hz, 1H), 4.07 - 3.78 (m, 4H), 2.55 (quin, J = 10.1 Hz, 1H), 2.36 - 2.20 (m, 2H), 2.18 - 2.08 (m, 1H), 1.82 (q, J = 9.8 Hz, 1H), 1.73 - 1.65 (m, 1H); LCMS (ESI) m/z: 381.1 [M+H]⁺.

The above racemic compound was separated using chiral HPLC to afford the enantiomers:

Compound 61: ¹H NMR (400 MHz, Chloroform-d) δ 9.11 (dd, J = 1.3, 4.9 Hz, 1H), 8.59 (s, 1H), 7.88 -7.75 (m, 2H), 7.69 (d, J = 8.2 Hz, 1H), 7.51 (dd, J = 5.0, 8.5 Hz, 1H), 5.36 (br. d, J = 3.7 Hz, 1H), 5.17 (br. d, J = 8.6 Hz, 1H), 4.38 (quin, J = 8.6 Hz, 1H), 4.07 - 3.79 (m, 4H), 2.55 (quin, J = 10.2 Hz, 1H), 2.36 -2.21 (m, 2H), 2.19 - 2.09 (m, 1H), 1.82 (q, J = 9.7 Hz, 1H), 1.75 - 1.68 (m, 1H); LCMS (ESI) m/z: 381.2 [M+H]⁺.

Compound 62: ¹H NMR (400 MHz, Chloroform-d) δ 9.19 - 9.05 (m, 1H), 8.59 (s, 1H), 7.81 (t, J = 9.3 Hz, 2H), 7.68 (d, J = 8.2 Hz, 1H), 7.51 (dd, J = 5.0, 8.5 Hz, 1H), 5.36 (br. s, 1H), 5.17 (br d, J = 7.5 Hz, 1H), 4.46 - 4.27 (m, 1H), 4.05 - 3.79 (m, 4H), 2.55 (quin, J = 10.1 Hz, 1H), 2.35 - 2.21 (m, 2H), 2.20 - 2.08 (m, 1H), 1.82 (q, J = 9.7 Hz, 1H), 1.75 - 1.66 (m, 1H); LCMS (ESI) m/z: 381.2 [M+H]⁺.

### Example 34: Preparation of 2-(5-Chloro-6-Methyl-2-Pyridyl)-N-[2-Hydroxy-2-(3-Pyridyl)ethyl]-N-Propyl-Acetamide (Compound 63)

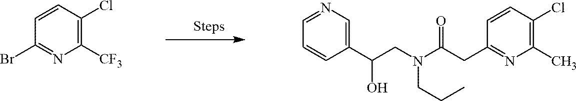 [SMILES: CCCN(CCc1ccc(Cl)c(Cl)c1)CC(O)c1cccnc1]

The compound 63 was synthesized similar to the protocol described for the compound 45, except that an achiral amine was used in the final amide coupling step. The compound 2-(5-chloro-6-methyl-2-pyridyl)-N-[2-hydroxy-2-(3-pyridyl)ethyl]-N-propyl-acetamide (26 mg, 74 µmol, 55%) was obtained as a pale yellow thick oil. ¹H NMR (400 MHz, Methanol-d₄) δ 8.75 (br. d, J = 9.4 Hz, 1H), 8.67 (s, 1H), 8.65 -8.54 (m, 1H), 8.52 - 8.41 (m, 1H), 8.05 - 7.83 (m, 2H), 7.51 - 7.36 (m, 1H), 5.04 (dt, J = 3.9, 8.3 Hz, 1H), 4.22 - 3.90 (m, 2H), 3.89 - 3.81 (m, 1H), 3.74 - 3.56 (m, 1H), 3.55 - 3.43 (m, 2H), 1.75 - 1.55 (m, 2H), 1.00 - 0.85 (m, 3H); LCMS (ESI) m/z: 368.1 [M+H]⁺.

### Example 35: Preparation of 2-(2-Chlorobenzothiophen-6-yl)-N-[(2S)-2-Hydroxy-2-(3-Pyridyl)Ethyl]-N-Propyl-Acetamide (Compound 67)

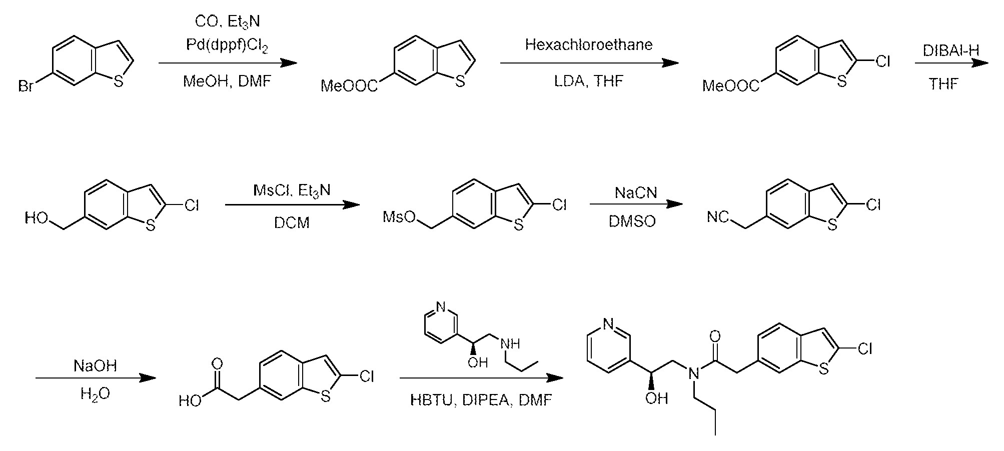 [SMILES: O=C(N[C@@H](Cc1ccc(F)cc1)C(=O)N1CCC(O)CC1)c1cc2cc(Cl)ccc2[nH]1]

### Step 1: Preparation of Methyl Benzothiophene-6-Carboxylate

To a solution of 6-bromobenzothiophene (3.1 g, 14.55 mmol) in dimethylformamide (40 mL) were added methanol (9.32 g, 291 mmol), [1,1′-bis(diphenylphosphino)ferrocene]dichloropalladium(II) (1.06 g, 1.45 mmol) and triethylamine (2.94 g, 29.10 mmol, 4.05 mL). The mixture was stirred under a CO atmosphere (50 psi) at 80° C. for 16 h. The reaction mixture was filtered and the filtrate was concentrated in vacuo. The crude product was purified by flash column (ISCO 40 g silica, 0-10% ethyl acetate in petroleum ether, gradient over 20 min) to yield methyl benzothiophene-6-carboxylate (2.7 g, 14.05 mmol, 97%) as a white solid.

### Step 2: Preparation of Methyl 2-Chlorobenzothiophene-6-Carboxylate

To a solution of methyl benzothiophene-6-carboxylate (500 mg, 2.60 mmol) in tetrahydrofuran (15 mL) was added (lithium diisopropylamine, 1.56 mL, 2 M in tetrahydrofuran) at -70° C. The reaction mixture was stirred at -70° C. for 0.5 h before hexachloroethane (1.23 g, 5.20 mmol) in tetrahydrofuran (5 mL) was added. The reaction was then stirred at -70° C. for 2 h. Water (20 mL) was added to the reaction, the reaction mixture was extracted with ethyl acetate (30 mL × 2). The combined organic layers were washed with brine (30 mL), dried over sodium sulfate, filtered and concentrated. The crude product was purified by flash column (ISCO 20 g silica, 0-10% ethyl acetate in petroleum ether, gradient over 20 min) to give methyl 2-chlorobenzothiophene-6-carboxylate (290 mg, 1.28 mmol, 49%) as a white solid.

### Step 3: Preparation of (2-Chlorobenzothiophen-6-yl)Methanol

To a solution of methyl 2-chlorobenzothiophene-6-carboxylate (200 mg, 882 µmol) in tetrahydrofuran (6 mL) was added diisobutylaluminum hydride (2.65 mL, 1 M in toluene) at -60° C. and mixture was warmed up and stirred at 20° C. for 1 h. HCl (15 mL, 1M) was added to the reaction and the aqueous layer was extracted with ethyl acetate (30 mL × 2). The combined organic layers were washed with brine (15 mL), dried over sodium sulfate, filtered and concentrated. The crude product was purified by flash column (ISCO 10 g silica, 0-50% ethyl acetate in petroleum ether, gradient over 20 min) to give (2-chlorobenzothiophen-6-yl)methanol (120 mg, 604 µmol, 68%) as a white solid. ¹H NMR (400 MHz, Chloroform-d) δ 7.73 (s, 1H), 7.65 (d, J = 8.2 Hz, 1H), 7.34 (dd, J = 1.2, 8.1 Hz, 1H), 7.17 (s, 1H), 4.80 (br. d, J = 4.4 Hz, 2H), 1.81 - 1.70 (m, 1H).

### Step 4: Preparation of (2-Chlorobenzothiophen-6-yl)Methyl Methanesulfonate

To a solution of (2-chlorobenzothiophen-6-yl)methanol (120 mg, 604 µmol) in dichloromethane (2 mL) were added triethylamine (92 mg, 906 µmol, 126 µL) and methanesulfonyl chloride (83 mg, 725 mmol) at 0° C. The mixture was stirred at 20° C. for 1 h, dichloromethane (30 mL) was added to the reaction and the organic phase was washed with saturated ammonium chloride (15 mL) and brine (15 mL). The combined organic layer was dried over sodium sulfate, filtered and concentrated to dryness. The crude (2-chlorobenzothiophen-6-yl)methyl methanesulfonate (160 mg) as a pale yellow oil and used directly in the next step without purification.

### Step 5: Preparation of 2-(2-chlorobenzothiophen-6-yl)acetonitrile.

To a solution of (2-chlorobenzothiophen-6-yl)methyl methanesulfonate (140 mg, 506 µmol) in dimethylsulfoxide (4 mL) was added sodium cyanide (74 mg, 1.52 mmol). The mixture was stirred at 20° C. for 1 h. Water (20 mL) was added to the reaction and the reaction mixture was extracted with ethyl acetate (30 mL × 2). The combined organic layers were washed with brine (15 mL) and dried over sodium sulfate and concentrated. The crude residue was purified by flash column (ISCO 4 g silica, 0-10% ethyl acetate in petroleum ether, gradient over 20 min) to give 2-(2-chlorobenzothiophen-6-yl)acetonitrile (80 mg, 385 µmol, 76%) as a pale yellow solid. ¹H NMR (400 MHz, Chloroform-d) δ 7.71 (s, 1H), 7.67 (d, J = 8.2 Hz, 1H), 7.29 (dd, J = 1.5, 8.3 Hz, 1H), 7.19 (s, 1H), 3.86 (s, 2H).

### Step 6: Preparation of 2-(2-Chlorobenzothiophen-6-yl)Acetic Acid

A mixture of 2-(2-chlorobenzothiophen-6-yl)acetonitrile (60 mg, 289 µmol) in sodium hydroxide (4 M, 361 µL) was stirred at 110° C. for 16 h. Water (5 mL) was added to the reaction, the mixture was acidified by 12 M HCl (1 mL) in 0° C., the mixture was extracted with ethyl acetate (10 mL × 2). The combined organic layers were washed with brine (15 mL) and dried over sodium sulfate and concentrated. The crude product 2-(2-chlorobenzothiophen-6-yl)acetic acid (65 mg, crude) was used directly in the next step without additional purification.

### Step 7: Preparation of 2-(2-Chlorobenzothiophen-6-yl)-N-[(2S)-2-Hydroxy-2-(3-Pyridyl)ethyl]-N-Propyl-Acetamide

To a solution of 2-(2-chlorobenzothiophen-6-yl)acetic acid (65 mg, 287 µmol) in dimethylformamide (2 mL) were added (1S)-2-(propylamino)-1-(3-pyridyl)ethanol (52 mg, 287 µmol), N,N,N′,N′-tetramethyl-O-(1H-benzotriazol-1-yl)uronium hexafluorophosphate (120 mg, 315 µmol) and diisopropylethylamine (111 mg, 860 µmol, 150 µL). The mixture was stirred at 20° C. for 0.5 h before it was filtered. The filtrate was purified by prep-HPLC (Waters Xbridge BEH C18 100 × 30 10 um column; 30-60% acetonitrile in an a 10 mM ammonium bicarbonate solution in water, 8 min gradient) to give 2-(2-chlorobenzothiophen-6-yl)-N-[(2S)-2-hydroxy-2-(3-pyridyl)ethyl]-N-propyl-acetamide (56 mg, 143 µmol, 49.94%) as a pale yellow thick oil. ¹H NMR (400 MHz, Chloroform-d) δ 8.62 - 8.55 (m, 1H), 8.52 (dd, J = 1.3, 4.8 Hz, 1H), 7.79 - 7.50 (m, 3H), 7.27 - 7.12 (m, 3H), 5.10 - 4.86 (m, 2H), 3.88 - 3.71 (m, 3H), 3.56 -3.47 (m, 1H), 3.35 - 3.17 (m, 1H), 3.07 (ddd, J = 6.3, 9.0, 14.9 Hz, 1H), 1.61 - 1.47 (m, 2H), 0.88 (t, J = 7.4 Hz, 3H); LCMS (ESI) m/z: 389.1 [M+H]⁺.

### Example 36: Preparation of Enantiomer 1 (Compound 69) and Enantiomer 2 (Compound 68) of N-[2-Hydroxy-2-Pyridazin-3-yl-Ethyl]-N-Propyl-2-[6-(Trifluoromethyl)-3-Pyridyl]Acetamide

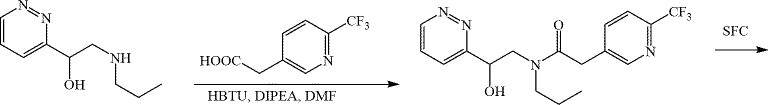

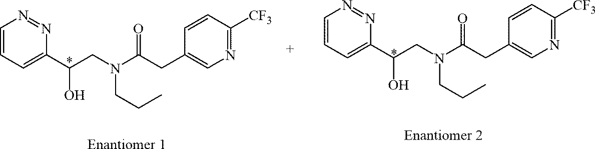

To a solution of 2-(propylamino)-1-pyridazin-3-yl-ethanol (250 mg, 1.38 mmol) in dimethylformamide (4 mL) were added 2-[6-(trifluoromethyl)-3-pyridyl]acetic acid (283 mg, 1.38 mmol), N,N,N′,N′-tetramethyl-O-(1H-benzotriazol-1-yl)uronium hexafluorophosphate (575 mg, 1.52 mmol) and diisopropylethylamine (535 mg, 4.14 mmol, 721 µL). The mixture was stirred at 20° C. for 1 h and filtered.

The filtrate was purified directly by prep-HPLC (Phenomenex Gemini-NX 150 × 30 5 um column; 10-40% acetonitrile in an a 10 mM ammonium bicarbonate solution in water, 8 min gradient) to give N-[2-hydroxy-2-pyridazin-3-yl-ethyl]-N-propyl-2-[6-(trifluoromethyl)-3-pyridyl]acetamide (240 mg) as a pale yellow thick oil. The racemic product was purified by preparative SFC DAICEL CHIRALCEL O (250 mm × 30 mm, 10 um) column, 40° C., eluting with 50% ethanol containing 0.1% ammonium hydroxide in a flow of 70 g/min CO2 at 100 bar) to give enantiomer 1 and 2.

Enantiomer 1: ¹H NMR (400 MHz, Chloroform-d) δ 9.09 (dd, J = 1.8, 4.8 Hz, 1H), 8.56 (s, 1H), 7.79 (d, J = 8.3 Hz, 2H), 7.71 - 7.61 (m, 1H), 7.48 (dd, J = 4.8, 8.3 Hz, 1H), 5.39 (d, J = 4.8 Hz, 1H), 5.32 - 5.19 (m, 1H), 4.07 - 3.87 (m, 2H), 3.85 - 3.75 (m, 2H), 3.52 - 3.29 (m, 2H), 1.78 - 1.68 (m, 2H), 1.01 - 0.85 (m, 3H); LCMS (ESI) m/z: 369.2 [M+H]⁺.

Enantiomer 2: ¹H NMR (400 MHz, Chloroform-d) δ 9.09 (dd, J = 1.8, 4.8 Hz, 1H), 8.56 (s, 1H), 7.79 (d, J = 8.3 Hz, 2H), 7.71 - 7.61 (m, 1H), 7.48 (dd, J = 4.8, 8.3 Hz, 1H), 5.39 (d, J = 4.8 Hz, 1H), 5.32 - 5.19 (m, 1H), 4.07 - 3.87 (m, 2H), 3.85 - 3.75 (m, 2H), 3.52 - 3.29 (m, 2H), 1.78 - 1.68 (m, 2H), 1.01 - 0.85 (m, 3H); LCMS (ESI) m/z: 369.2 [M+H]⁺.

The following enantiomerically pure compounds were obtained by chiral HPLC conditions described above.

| Name | Structure | NMR, MS, Retention time | Compd# |
|---|---|---|---|
| N-[2-hydroxy-2-(4-methyl-3-pyridyl)ethyl] -N-(oxetan-3-ylmethyl)-2-[6-(trifluoromethyl)-3-pyridyl]acetamide (enantiomer 2) | 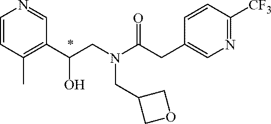 | ¹H NMR (400 MHz, Chloroform-d) δ 8.73 - 8.60 (m, 1H), 8.57 - 8.47 (m, 1H), 8.41 - 8.28 (m, 1H), 7.77 (br. t, J = 9.3 Hz, 1H), 7.70 - 7.58 (m, 1H), 7.11 - 7.01 (m, 1H), 5.27-5.15 (m, 1H), 5.16 (dd, J = 2.6, 9.3 Hz, 1H), 4.83 (dt, J = 3.3, 6.9 Hz, 1H), 4.76 - 4.68 (m, 1H), 4.52 - 4.36 (m, 2H), 4.06 - 3.91 (m, 5H), 3.89 - 3.84 (m, 1H), 3.49 - 3.29 (m, 2H), 2.39 (s, 3H); LCMS (ESI) m/z: 410.2 [M+H]⁺ | 88 |
| N-[2-hydroxy-2-(4-methyl-3-pyridyl)ethyl] -N-(oxetan-3-ylmethyl)-2-[6-(trifluoromethyl)-3-pyridyl]acetamide (enantiomer 1) | 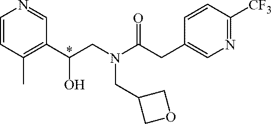 | ¹H NMR (400 MHz, Chloroform-d) δ 8.73 - 8.60 (m, 1H), 8.57 - 8.47 (m, 1H), 8.41 - 8.28 (m, 1H), 7.77 (br. t, J = 9.3 Hz, 1H), 7.70 - 7.58 (m, 1H), 7.11 - 7.01 (m, 1H), 5.27 (dd, J = 2.2, 8.8 Hz, 0.6H), 5.16 (dd, J = 2.6, 9.3 Hz, 0.4H), 4.83 (dt, J = 3.3, 6.9 Hz, 1H), 4.76 - 4.68 (m, 1H), 4.52 - 4.36 (m, 2H), 4.06 - 3.91 (m, 1H), 3.89 - 3.84 (m, 1H), 3.83 - 3.68 (m, 2H), 3.49 -3.29 (m, 2H), 3.28 - 3.15 (m, 1H), 2.39 (s, 3H); LCMS (ESI) m/z: 410.2 [M+H]⁺ | 89 |
| N-[2-hydroxy-2-(4-isopropyl-3-pyridyl)ethyl]-N-(oxetan-3-ylmethyl)-2-[6-(trifluoromethyl)-3-pyridyl]acetamide (enantiomer 1) | 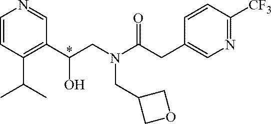 | ¹H NMR (400 MHz, Chloroform-d) δ 8.78 - 8.65 (m, 1H), 8.64 - 8.42 (m, 2H), 7.89 - 7.75 (m, 1H), 7.74 - 7.61 (m, 1H), 7.25 - 7.15 (m, 1H), 5.47 -5.21 (m, 1H), 4.92 - 4.69 (m, 2H), 4.57 - 4.36 (m, 2H), 4.17 - 3.66 (m, 5H), 3.56 - 3.03 (m, 3H), 1.36 - 1.16 (m, 6H); LCMS (ESI) m/z: 438.2 [M+H]⁺; (Rt: 1.602 min). | 91 |
| N-[2-hydroxy-2-(4-isopropyl-3-pyridyl)ethyl]-N-(oxetan-3-ylmethyl)-2-[6-(trifluoromethyl)-3-pyridyl]acetamide (enantiomer 2) | 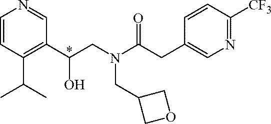 | ¹H NMR (400 MHz, Chloroform-d) δ 8.78 - 8.66 (m, 1H), 8.64 - 8.43 (m, 2H), 7.89 - 7.75 (m, 1H), 7.74 - 7.61 (m, 1H), 7.26 - 7.16 (m, 1H), 5.45 -5.25 (m, 1H), 4.92 - 4.70 (m, 2H), 4.57 - 4.35 (m, 2H), 4.14 - 3.67 (m, 5H), 3.54 - 3.05 (m, 4H), 1.37 - 1.14 (m, 6H); LCMS (ESI) m/z: 438.2 [M+H]⁺; (Rt: 1.691 min). | 92 |

### Example 37: Characterization of Data for Further Compounds

Compounds 85 and 87 below were prepared following procedure similar to Compound 8.

| # | LCMS | NMR |
|---|---|---|
| 85 | LCMS (ESI) m/z: 381.2 [M+H]⁺ | ¹H NMR (400 MHz, Chloroform-d) δ ppm 1.63 - 1.85 (m, 3 H) 2.09 - 2.29 (m, 3 H) 2.40 (quin, J = 9.98 Hz, 1 H) 3.82 - 3.93 (m, 4 H) 4.30 - 4.41 (m, 1 H) 4.98 - 5.04 (m, 1 H) 7.69 (d, J = 7.94 Hz, 1 H) 7.82 (br. d, J = 8.38 Hz, 1 H) 8.53 (s, 2 H) 8.60 (s, 1 H) 8.88 (s, 1 H) |
| 87 | LCMS (ESI) m/z: 395.2 [M+H]⁺ | ¹H NMR (400 MHz, Chloroform-d) δ 9.08 (dd, J = 1.3, 4.9 Hz, 1H), 8.55 (s, 1H), 7.79 -7.73 (m, 2H), 7.66 (d, J = 8.1 Hz, 1H), 7.46 (dd, J = 5.0, 8.5 Hz, 1H), 5.36 (br. s, 1H), 5.21 (dd, J = 2.7, 7.2 Hz, 1H), 4.05 - 3.67 (m, 4H), 3.58 - 3.49 (m, 1H), 3.46 - 3.35 (m, 1H), 2.68 (spt, J = 7.8 Hz, 1H), 2.22 - 2.06 (m, 2H), 2.02 - 1.72 (m, 4H) |

### Example 38: Characterization of Data for Further Compounds

Compounds 96, 97, 113, 116, and 129 below were prepared following procedure similar to Compound 1.

| # | LCMS | NMR |
|---|---|---|
| 96 | LCMS (ESI) m/z: 368.0 [M+H]⁺ | ¹H NMR (400 MHz, Chloroform-d) δ 8.78 (s, 1H), 8.44 - 8.37 (m, 2H), 7.32 (d, J = 8.2 Hz, 1H), 7.28 - 7.23 (m, 1H), 6.99 (dd, J = 1.9, 8.3 Hz, 1H), 5.00 (dd, J = 3.3, 7.0 Hz, 1H), 3.79 - 3.64 (m, 2H), 3.65 - 3.50 (m, 3H), 3.29 - 3.07 (m, 2H), 1.61 - 1.44 (m, 2H), 0.84 (t, J = 7.4 Hz, 3H) |
| 97 | LCMS (ESI) m/z: 368.0 [M+H]⁺ | ¹H NMR (400 MHz, Chloroform-d) δ 9.10 (dd, J = 1.3, 4.9 Hz, 1H), 7.82 (dd, J = 1.2, 8.4 Hz, 1H), 7.49 (dd, J = 5.0, 8.4 Hz, 1H), 7.42 (d, J = 8.3 Hz, 1H), 7.33 (d, J = 1.8 Hz, 1H), 7.07 (dd, J = 1.9, 8.3 Hz, 1H), 5.65 (br. s, 1H), 5.27 (dd, J = 2.6, 7.0 Hz, 1H), 4.00 - 3.87 (m, 2H), 3.68 (s, 2H), 3.42 - 3.28 (m, 1H), 3.29 - 3.16 (m, 1H), 1.70 -1.58 (m, 2H), 1.00 - 0.83 (m, 3H) |
| 113 | LCMS (ESI) m/z: 369.0 [M+H]⁺ | ¹H NMR (400 MHz, Chloroform-d) δ 8.89 - 8.79 (m, 1H), 8.62 - 8.56 (m, 1H), 8.54 -8.45 (m, 1H), 7.86 - 7.79 (m, 1H), 7.71 - 7.62 (m, 1H), 5.15 - 5.06 (m, 1H), 5.04 -4.95 (m, 1H), 3.91 - 3.61 (m, 4H), 3.49 (m, 2H), 1.65 - 1.60 (m, 2H), 1.02 - 0.86 (m, 3H) |
| 116 | LCMS (ESI) m/z: 418.2 [M+H]⁺ | ¹H NMR (400 MHz, Chloroform-d) δ 9.08 (br. d, J = 3.5 Hz, 1H), 7.84 - 7.75 (m, 1H), 7.47 (dd, J = 4.9, 8.4 Hz, 1H), 7.34 (br. d, J = 1.7 Hz, 1H), 7.30 (br. s, 1H), 7.15 (br. d, J = 8.3 Hz, 1H), 5.58 (br. s, 1H), 5.26 (br. s, 1H), 4.00 - 3.88 (m, 2H), 3.69 (s, 2H), 3.43 - 3.18 (m, 2H), 1.70 - 1.62 (m, 2H), 0.94 (t, J = 7.4 Hz, 3H) |
| 129 | LCMS (ESI) m/z: 394.2 [M+H]⁺ | ¹H NMR (400 MHz, Chloroform-d) δ 8.89 (d, J = 1.0 Hz, 1H), 8.55 - 8.49 (m, 2H), 7.29 (s, 1H), 7.28 - 7.27 (m, 1H), 5.37 (d, J = 4.9 Hz, 1H), 5.13 - 5.06 (m, 1H), 3.89 -3.77 (m, 2H), 3.55 (quin, J = 8.3 Hz, 1H), 3.39 - 3.25 (m, 2H), 3.23 - 3.15 (m, 2H), 3.12 - 3.01 (m, 2H), 1.71 - 1.61 (m, 2H), 0.94 (t, J = 7.4 Hz, 3H) |

### Example 39: Inhibition of CYP51A1 by Compounds of the Invention

Method: Recombinant human CYP51A1 (lanosterol-14a-demethylase) enzyme was coexpressed with CYP reductase in bacterial membranes and the fluorescent substrate BOMCC (a nonnatural substrate that causes increases in fluorescence upon CYP51A1-dependent demethylation) was used to obtain 8-point dose concentration-response curves for each compound.

Results: As shown in Table 4, the compounds of the invention inhibit CYP51A1.

TABLE 4

| # | CYP51 IC₅₀ (µM) | # | CYP51 IC₅₀ (µM) | # | CYP51 IC₅₀ (µM) | # | CYP51 IC₅₀ (µM) | # | CYP51 IC₅₀ (µM) | # | CYP51 IC₅₀ (µM) |
|---|---|---|---|---|---|---|---|---|---|---|---|
| 1 | +++ | 26 | ++ | 51 | + | 76 | +++ | 100 | +++ | 124 | +++ |
| 2 | +++ | 27 | +++ | 52 | ++ | 77 | +++ | 101 | + | 125 | +++ |
| 3 | ++ | 28 | + | 53 | ++ | 78 | ++ | 102 | + | 126 | ++ |
| 4 | ++ | 29 | + | 54 | + | 79 | + | 103 | + | 127 | + |
| 5 | + | 30 | + | 55 | + | 80 | ++ | 104 | + | 128 | +++ |
| 6 | +++ | 31 | +++ | 556 | + | 81 | + | 105 | +++ | 129 | ++ |
| 7 | +++ | 32 | ++ | 57 | + | 82 | ++ | 106 | ++ | 130 | +++ |
| 8 | +++ | 33 | + | 58 | + | 83 | ++ | 107 | +++ | 131 | +++ |
| 9 | ++ | 34 | ++ | 59 | + | 84 | + | 108 | + | 132 | + |
| 10 | + | 35 | ++ | 60 | ++ | 85 | ++ | 109 | +++ | 133 | ++ |
| 11 | +++ | 36 | + | 61 | ++ | 86 | ++ | 110 | ++ | 134 | +++ |
| 12 | ++ | 37 | + | 62 | + | 87 | ++ | 111 | + | 135 | +++ |
| 13 | + | 38 | ++ | 63 | + | 88 | +++ | 112 | ++ | 136 | +++ |
| 14 | + | 39 | + | 64 | +++ | 89 | + | 113 | + | 137 | +++ |
| 15 | + | 40 | ++ | 65 | + | 90 | +++ | 114 | + | 138 | ++ |
| 16 | +++ | 41 | + | 66 | ++ | 91 | +++ | 115 | + | 139 | +++ |
| 17 | +++ | 42 | +++ | 67 | +++ | 92 | +++ | 116 | ++ | 140 | +++ |
| 18 | +++ | 43 | + | 68 | + | 93 | + | 117 | + | 141 | +++ |
| 19 | + | 44 | + | 69 | +++ | 94 | ++ | 118 | + | 142 | +++ |
| 20 | ++ | 45 | +++ | 70 | +++ | 95 | ++ | 119 | +++ | 143 | +++ |
| 21 | +++ | 46 | + | 71 | +++ | 96 | ++ | 120 | +++ | 144 | +++ |
| 22 | ++ | 47 | ++ | 72 | ++ | 97 | +++ | 121 | + | 145 | ++ |
| 23 | +++ | 48 | + | 73 | ++ | 98 | +++ | 122 | + | 146 | +++ |
| 24 | ++ | 49 | + | 74 | +++ | 99 | ++ | 123 | + | 147 | +++ |
| 25 | ++ | 50 | +++ | 75 | + | | | | | | |

“+++” = ≤0.1 µM; “++” = >0.1 µM to ≤1 µM; “+” = >1 µM

### Example 40. Inhibition of CYP51A1 Modulates TDP-43 Aggregation

### Introduction

Amyotrophic lateral sclerosis (ALS), also known as Lou Gehrig’s disease, is an aggressive, debilitating disease in which affected patients succumb within two to five years after diagnosis. ALS presents with heterogeneous clinical features but has a common underlying pathology of motor neuron loss that limits the central nervous system’s ability to effectively regulate voluntary and involuntary muscle activity. Additionally, without neuronal trophic support muscles being to atrophy, further exacerbating motor deterioration. Cellular and tissue degeneration results in motor impairment such as fasciculations and weakening in the arms, legs and neck, difficulty swallowing, slurred speech and ultimately failure of the diaphragm muscles that control breathing.

At the cellular level, 97% of all ALS cases have the common pathological feature of misfolded and aggregated TAR-DNA binding protein (TDP)-43 in spinal motor neuron inclusions. TDP-43 is a DNA/RNA binding protein involved in RNA splicing and is typically localized to the nucleus but can be translocated to the cytoplasm under conditions of cell stress. Nuclear clearing and cytoplasmic accumulation of misfolded and aggregated TDP-43 are hallmarks of degenerating motor neurons in ALS, but it remains unclear if mechanism of toxicity is due to aggregation-dependent loss of TDP-43 function or if the aggregates acquire toxic gain of function. Aggregates of TDP-43 accumulate in discrete cellular domains known as stress granules, which are also enriched with translationally inactive mRNAs. Stress granules are observed in multiple cellular types and are thought to be directly related to TDP-43-dependent toxicity in ALS and FTD. Dysfunction in DNA/RNA binding protein activity plays a crucial role in susceptible motor neurons in ALS, as familial cases have also been traced to mutations in the protein Fused in Sarcoma (FUS), a DNA/RNA binding protein that recently has been shown to be involved in gene silencing. Preclinical studies suggest that FUS mutations promote a toxic gain of function that may be causative in motor neuron degeneration.

Mutations in the TDP-43 gene (TARDBP) have also been causally linked to familial forms of ALS. A common TDP-43 mutation is known as Q331K, in which glutamine (Q) 331 has been mutated to a lysine (K). This mutation results in a TDP-43 protein that is more aggregation prone and exhibits enhanced toxicity. A recent study has also demonstrated that the Q331K mutation can confer a toxic gain of function in a TDP-43 knock-in mouse, which exhibits cognitive deficits and histological abnormalities similar to that which occurs in frontotemporal dementia (FTD). FTD refers to a group of degenerative disorders that are characterized by atrophy in the frontal and temporal cortices due to progressive neuron loss. Due to the functional nature of the brain regions impacted in FTD, the most common symptoms involve noticeable alterations in personality, behavior and linguistic ability and can also present with loss of speech. The pathological basis of FTD appears to be multifactorial involving mutations in genes such as C9orf72, progranulin (GRN) and MAPT, but intracellular inclusions of aggregated TDP-43, FUS and tau have been observed. Although ALS and FTD may have different genetic and molecular triggers and occur in different cell types, similar protein misfolding and degenerative mechanisms may operate in multiple diseases.

The toxic gain of function features of TDP-43 can be faithfully recapitulated in the simple model organism, budding yeast, where the protein also localizes to stress granules. Human disease mutations in TDP-43 enhance toxicity and yeast genetic screens have revealed key connections that are conserved to humans. The yeast model thus provides a robust cell-based screening platform for small molecules capable of ameliorating toxicity. To validate compounds from such phenotypic screens, it is imperative to test compounds in a mammalian neuronal context. In an effort to develop TDP-43-related mammalian models of neuron loss that occurs in ALS and FTD, primary cultures of rat cortical neurons were transfected with human wild type or Q331K mutant TDP-43. These cells were compared to cells which received an empty expression vector control. Validation studies have demonstrated that cells expressing either wild type or Q331K TDP-43 have are more susceptible to dying over time in culture. In the experiments described in this example, this model system is used to interrogate new therapeutic approaches to ameliorate TDP-43 toxicity.

### Results

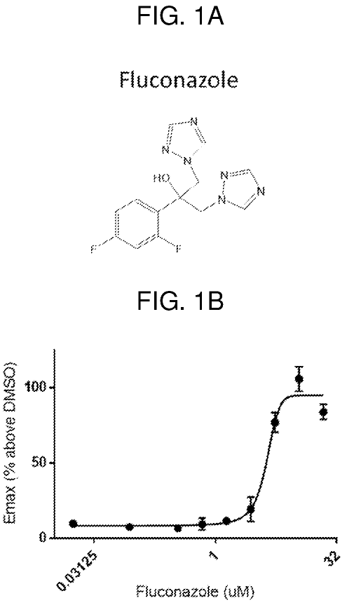

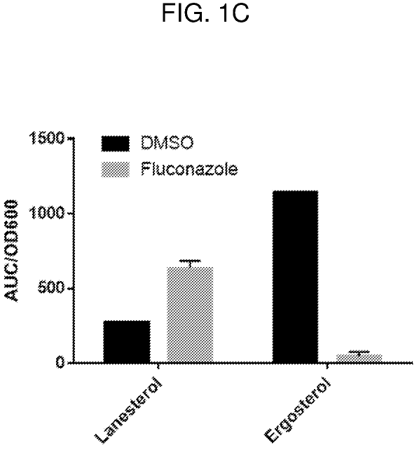

From the TDP-43 yeast model, a compound with known mode of action was identified that restored viability to TDP-43-expressing yeast (FIG. 1A). Fluconazole is an antifungal known to inhibit Erg11, the yeast lanosterol 14-alpha demethylase (FIG. 1B). Inhibition of Erg11 reduces ergosterol synthesis (yeast equivalent of cholesterol), while increasing lanosterol levels, the substrate of Erg11 (FIG. 1C). The human homolog of Erg11 is Cyp51A1, a member of the cytochrome P450 superfamily of enzymes but does not appear to have a role in detoxification of xenobiotics. CYP51A1 has also been known as lanosterol 14-alpha demethylase, which describes its function in removing the 14-alpha-methyl group from lanosterol to generate 4,4-dimethylcholesta-8(9), 14, 24-trien-3β-ol, which is a critical step in the cholesterol biosynthetic pathway.

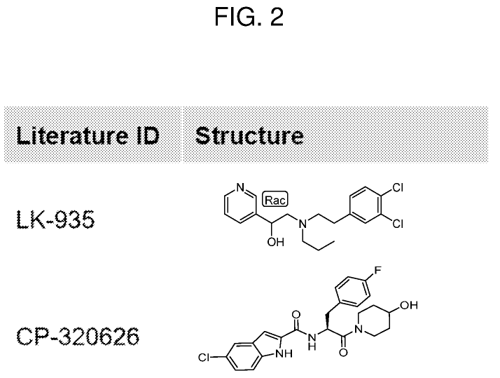

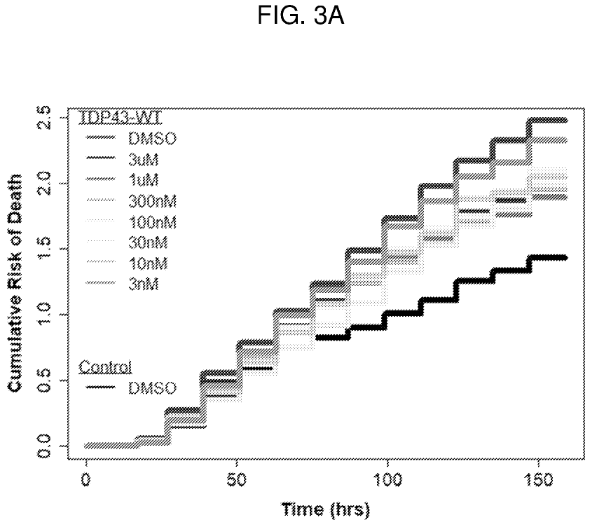

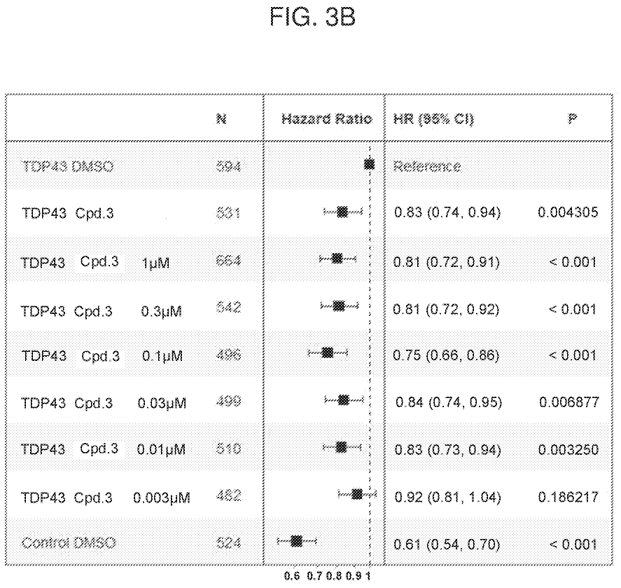

To evaluate the potential role of CYP51A1 in TDP-43 pathology, the aforementioned primary rat cortical neuron TDP-43 models were utilized to test the efficacy of published inhibitors (FIG. 2). Rat cortical neurons transfected with wild type human TDP-43 exhibited a significant reduction in survival compared to neurons transfected with empty vector control, and this reduction in survival was partially alleviated by treatment with compound A (FIGS. 3A and 3B). Compound A has the structure:

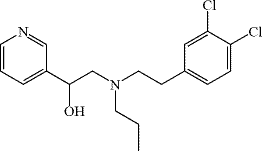

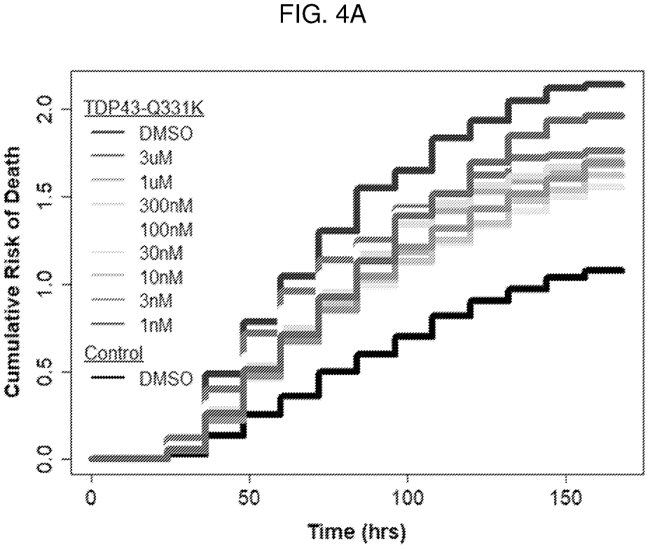

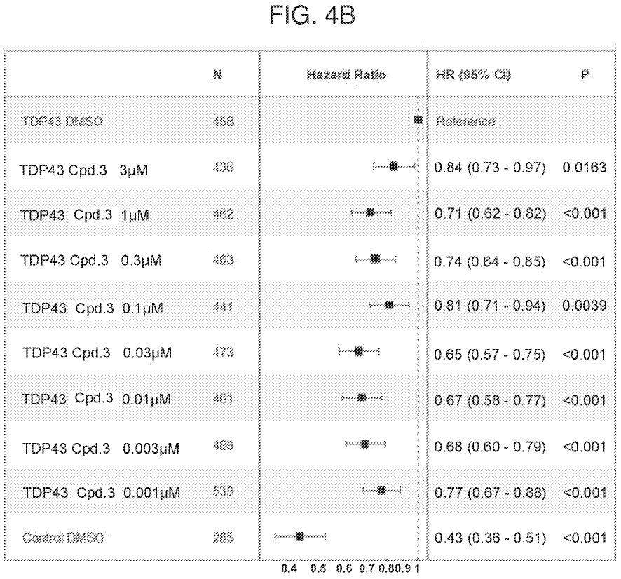

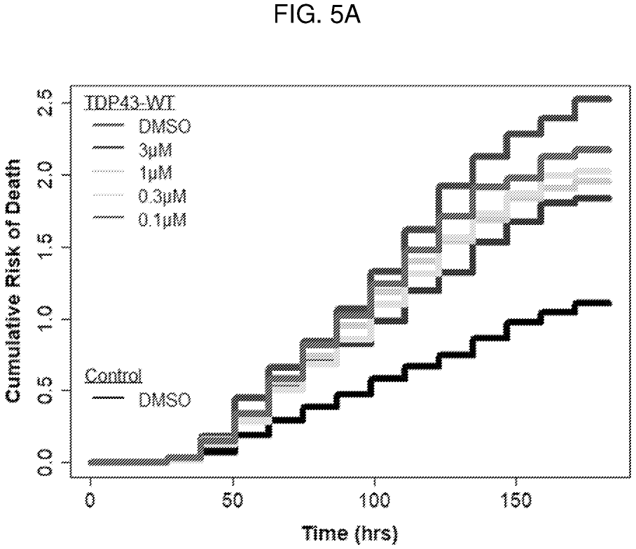

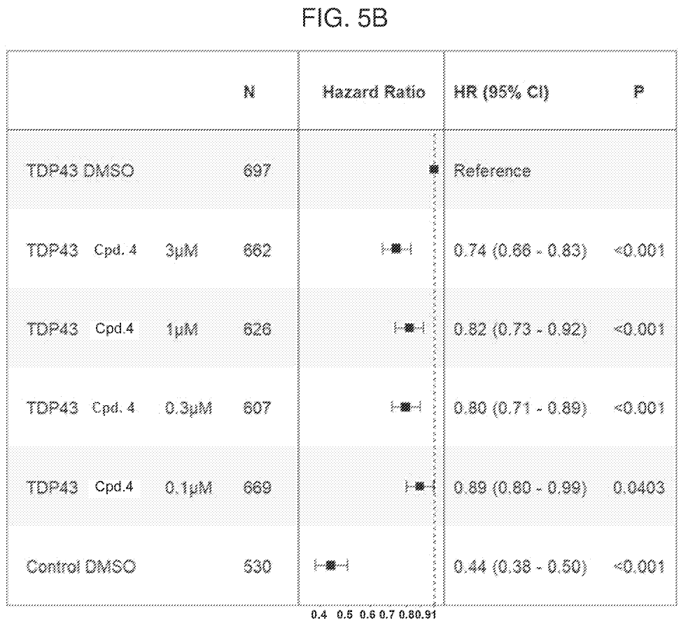

A similar survival befit was conferred by compound A when applied to cells transfected with Q331K mutant TDP-43 (FIGS. 4A and 4B). A similar effect in rescuing a survival deficit was observed for a structurally differentiated compound, compound B, when applied to cells transfected with wild-type TDP-43 (FIGS. 5A and 5B). Compound B has the structure:

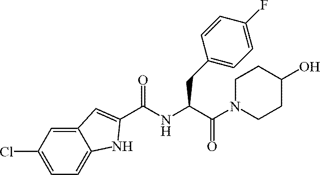

These studies demonstrate that inhibition of Erg11 in yeast and inhibition of Cyp51A1 has a beneficial effect of rescuing cells from wild type and mutant TDP-43 toxicity and promotes cell survival. This is the first demonstration that inhibition of CYP51A1 is beneficial in treating and preventing TDP-43 pathological processes and represents a novel therapeutic approach for the treatment of ALS.

### Other Embodiments

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

Other embodiments are in the claims.

1. A compound, or a pharmaceutically acceptable salt thereof, having the structure: 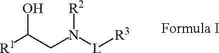
wherein R¹ has the structure: 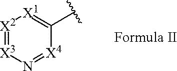
X¹, X², X³, and X⁴ are, independently, N or CR⁴, wherein at least three of X¹, X², X³, and X⁴ are CR⁴;
R² is optionally substituted C₁-C₆ alkyl, optionally substituted C₁-C₆ alkoxy, optionally substituted C₃-C₈ cycloalkyl, optionally substituted C₂-C₉ heterocylcyl, optionally substituted C₂-C₉ heteroaryl, optionally substituted C₁-C₆ alkyl C₃-C₈ cycloalkyl, optionally substituted C₁-C₆ alkyl C₂-C₉ heterocylcyl, optionally substituted C₁-C₆ alkyl C₂-C₉ heteroaryl, or optionally substituted C₁-C₆ alkyl C₆-C₁₀ aryl;
R³ is optionally substituted C₁-C₆ alkyl, optionally substituted C₁-C₆ alkoxy, optionally substituted C₃-C₈ cycloalkyl, optionally substituted C₂-C₉ heterocylcyl, optionally substituted C₂-C₉ heteroaryl, optionally substituted C₁-C₆ alkyl C₃-C₈ cycloalkyl, optionally substituted C₁-C₆ alkyl C₂-C₉ heterocylcyl, optionally substituted C₁-C₆ alkyl C₂-C₉ heteroaryl, or optionally substituted C₁-C₆ alkyl C₆-C₁₀ aryl, wherein if R² is methyl then R³ is optionally substituted C₁-C₆ alkyl, optionally substituted C₁-C₆ alkoxy, optionally substituted C₃-C₈ cycloalkyl, or optionally substituted C₁-C₆ alkyl C₃-C₈ cycloalkyl;
each R⁴ is, independently, hydrogen, hydroxy, halo, cyano, optionally substituted amino, optionally substituted C₁-C₆ alkyl, or optionally substituted C₁-C₆ alkoxy; and
L is —C(O)— or —SO₂—.

2. The compound of claim 1, wherein L is —C(O)—.

3. The compound of claim 1, wherein L is —SO₂—.

4. The compound of any one of claims 1 to 3, wherein X¹, X², X³, and X⁴ are CR⁴.

5. The compound of claim 4, wherein R¹ has the structure: 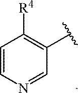
.

6. The compound of claim 5, wherein R⁴ is optionally substituted C₁-C₆ alkyl.

7. The compound of claim 6, wherein optionally substituted C₁-C₆ alkyl is methyl or iso-propyl.

8. The compound of claim 5, wherein R⁴ is optionally substituted amino.

9. The compound of claim 8, wherein optionally substituted amino is —NH₂.

10. The compound of claim 5, wherein R⁴ is optionally substituted C₁-C₆ alkoxy.

11. The compound of claim 10, wherein optionally substituted C₁-C₆ alkoxy is methoxy.

12. The compound of claim 4, wherein R¹ has the structure: 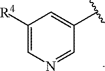
.

13. The compound of claim 12, wherein R⁴ is halo.

14. The compound of claim 13, wherein halo is fluoro, bromo, or chloro.

15. The compound of claim 12, wherein R⁴ is optionally substituted C₁-C₆ alkyl.

16. The compound of claim 15, wherein optionally substituted C₁-C₆ alkyl is —C(O)CH₃ or 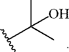
.

17. The compound of claim 12, wherein R⁴ is cyano.

18. The compound of claim 4, wherein R¹ has the structure: 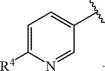
.

19. The compound of claim 18, wherein R⁴ is optionally substituted C₁-C₆ alkyl.

20. The compound of claim 19, wherein optionally substituted C₁-C₆ alkyl is trifluoromethyl.

21. The compound of claim 18, wherein R⁴ is hydroxy.

22. The compound of claim 18, wherein R⁴ is optionally substituted C₁-C₆ alkoxy.

23. The compound of claim 22, wherein optionally substituted C₁-C₆ alkoxy is methoxy.

24. The compound of any one of claims 1 to 3, wherein X¹ is N and X², X³, and X⁴ are CR⁴.

25. The compound of claim 24, wherein R⁴ is hydrogen.

26. The compound of any one of claims 1 to 3, wherein X¹, X², X⁴ are CR⁴, and X³ is N.

27. The compound of claim 26, wherein R⁴ is hydrogen.

28. The compound of any one of claims 1 to 3, wherein X¹, X², X³ are CR⁴, and X⁴ is N.

29. The compound of claim 28, wherein R⁴ is hydrogen.

30. The compound of any one of claims 1 to 29, wherein R² is optionally substituted C₁-C₆ alkyl.

31. The compound of claim 30, wherein optionally substituted C₁-C₆ alkyl is ethyl, propyl, iso-propyl, or —C(O)CH₂CH₃.

32. The compound of claim 31, wherein optionally substituted C₁-C₆ alkyl is propyl.

33. The compound of any one of claims 1 to 32, wherein R³ is optionally substituted C₁-C₆ alkyl C₆-C₁₀ aryl.

34. The compound of claim 33, wherein optionally substituted C₁-C₆ alkyl C₆-C₁₀ aryl is 3,4-chloro-benzyl, 2-fluoro-4-chloro-benzyl, 3-fluoro-4-trifluoromethyl-benzyl, 3-chloro-4-methyl-benzyl, 3-chloro-4-trifluoromethoxy-benzyl, 3-fluoro-4-difluoromethyl-benzyl, or napthyl.

35. The compound of claim 34, wherein optionally substituted C₁-C₆ alkyl C₆-C₁₀ aryl is 3,4-chloro-benzyl.

36. The compound of any one of claims 1 to 32, wherein R³ is optionally substituted C₁-C₆ alkyl C₂-C₉ heteroaryl.

37. The compound of claim 36, wherein optionally substituted C₁-C₆ alkyl C₂-C₉ heteroaryl is 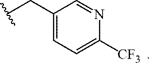
.

38. The compound of any one of claims 1 to 32, wherein R³ is optionally substituted C₃-C₈ cycloalkyl.

39. The compound of claim 38, wherein R³ is 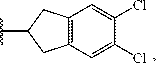 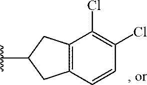 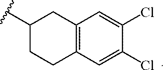
.

40. The compound of claim 39, wherein R³ is 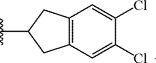
.

41. A compound, or pharmaceutically acceptable salt thereof, having the structure of any one of compounds 1-147 in Table 1.

42. A pharmaceutical composition comprising a compound of any one of claims 1 to 41 and a pharmaceutically acceptable excipient.

43. A method of treating a neurological disorder in a subject in need thereof, the method comprising administering an effective amount of a compound, or pharmaceutically acceptable salt thereof, of any one of claims 1 to 41 or a pharmaceutical composition of claim 42.

44. A method of inhibiting toxicity in a cell related to a protein, the method comprising administering an effective amount of a compound of any one of claims 1 to 41 or a pharmaceutical composition of claim 42.

45. The method of claim 44, wherein the toxicity is TDP-43-related toxicity.

46. The method of claim 44 or 45, wherein the cell is a mammalian neural cell.

47. A method of treating a CYP51A1-associated disorder in a subject in need thereof, the method comprising administering an effective amount of a compound, or pharmaceutically acceptable salt thereof, of any one of claims 1 to 41 or a pharmaceutical composition of claim 42.

48. The method of claim 47, wherein the CYP51A1-associated disorder is ALS.

49. A method of inhibiting CYP51A1, the method comprising contacting a cell with an effective amount of a compound of any one of claims 1 to 41 or a pharmaceutical composition of claim 42.


SEQUENCE LISTING (4 sequences)

SEQ ID NO: 1 - protein, 503 aa, Homo sapiens [sequence omitted]

SEQ ID NO: 2 - RNA, 1530 nt, Homo sapiens
atggcggcggcggctgggatgctgctgctgggcttgctgcaggcgggtgggtcggtgctgggccaggcgatggagaaggtgacaggcggcaacctcttgtccatgctgctgatcgcctgcgccttcaccctcagcctggtctacctgatccgtctggccgccggccacctggtccagctgcccgcaggggtgaaaagtcctccatacattttctccccaattccattccttgggcatgccatagcatttgggaaaagtccaattgaatttctagaaaatgcatatgagaagtatggacctgtatttagttttaccatggtaggcaagacatttacttaccttctggggagtgatgctgctgcactgctttttaatagtaaaaatgaagacctgaatgcagaagatgtctacagtcgcctgacaacacctgtgtttgggaagggagttgcatacgatgtgcctaatccagttttcttggagcagaagaaaatgttaaaaagtggccttaacatagcccactttaaacagcatgtttctataattgaaaaagaaacaaaggaatactttgagagttggggagaaagtggagaaaaaaatgtgtttgaagctctttctgagctcataattttaacagctagccattgtttgcatggaaaggaaatcagaagtcaactcaatgaaaaggtagcacagctgtatgcagatttggatggaggtttcagccatgcagcctggctcttaccaggttggctgcctttgcctagtttcagacgcagggacagagctcatcgggaaatcaaggatattttctataaggcaatccagaaacgcagacagtctcaagaaaaaattgatgacattctccaaactttactagatgctacatacaaggatgggcgtcctttgactgatgatgaagtagcagggatgcttattggattactcttggcagggcagcatacatcctcaactactagtgcttggatgggcttctttttggccagagacaaaacacttcaaaaaaaatgttatttagaacagaaaacagtctgtggagagaatctgcctcctttaacttatgaccagctcaaggatctaaatttacttgatcgctgtataaaagaaacattaagacttagacctcctataatgatcatgatgagaatggccagaactcctcagactgtggcagggtataccattcctccaggacatcaggtgtgtgtttctcccactgtcaatcaaagacttaaagactcatgggtagaacgcctggactttaatcctgatcgctacttacaggataacccagcatcaggggaaaagtttgcctatgtgccatttggagctgggcgtcatcgttgtattggggaaaattttgcctatgttcaaattaagacaatttggtccactatgcttcgtttatatgaatttgatctcattgatggatactttcccactgtgaattatacaactatgattcacacccctgaaaacccagttatccgttacaaacgaagatcaaaatga

SEQ ID NO: 3 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 4 - RNA, 1245 nt, Homo sapiens
atgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtag